(19) United States
(12) Patent Application Publication
(10) Pub. No.: US 2024/0238371 A1
(43) Pub. Date: Jul. 18, 2024

(54) MATERIALS AND METHODS FOR EXTRACELLULAR VESICLE-ASSOCIATED DISEASES

(71) Applicant: THE BRIGHAM AND WOMEN'S HOSPITAL, INC., Boston, MA (US)

(72) Inventors: Shinsuke ITO, Tokyo (JP); Elena AIKAWA, Chestnut Hill, MA (US); Masanori AIKAWA, Chestnut Hill, MA (US)

(73) Assignee: THE BRIGHAM AND WOMEN'S HOSPITAL, INC., Boston, MA (US)

(21) Appl. No.: 18/363,416

(22) Filed: Aug. 1, 2023

Related U.S. Application Data
(63) Continuation of application No. 16/753,359, filed on Apr. 3, 2020, now Pat. No. 11,833,187.
(60) Provisional application No. 62/575,762, filed on Oct. 23, 2017.

(51) Int. Cl. A61K 38/17; A61K 47/65; G01N 33/68

(52) U.S. Cl. CPC A61K 38/1741; A61K 47/65; G01N 33/6803

(57) ABSTRACT

Sortilin is a sorting receptor that directs target proteins to the secretary or endocytic compartments of cells that is found in both extracellular vesicles and cells. Provided herein are methods and compositions for decreasing or inhibiting trafficking of sortilin to an extracellular vesicle, for example by inhibiting the formation of intermolecular sortilin dimers.

### CROSS-REFERENCE TO RELATED APPLICATIONS

This application is a continuation under 35 U.S.C. § 120 of co-pending U.S. application Ser. No. 16/753,359, filed Apr. 3, 2020, which is a 371 National Phase Entry Application under 35 U.S.C. § 371 of International Application No. PCT/US2018/057016, filed on Oct. 23, 2018, which claims benefit under 35 U.S.C. § 119(e) of U.S. Provisional Application No. 62/575,762 filed Oct. 23, 2017, the contents of each of which are incorporated herein by reference in their entireties.

### SEQUENCE LISTING

The instant application contains a Sequence Listing which has been submitted electronically in XML format and is hereby incorporated by reference in its entirety. Said XML copy, created on Jul. 31, 2023, is named 043213-093350USC1_SL.xml and is 103,270 bytes in size.

### TECHNICAL FIELD

The technology provided herein relates to methods and compositions for decreasing or inhibiting trafficking of sortilin to an extracellular vesicle.

### BACKGROUND

Extracellular vesicles (EVs) play a critical role in intercellular communication by transferring microRNAs, lipids, and proteins to neighboring cells. Sortilin, a sorting receptor that directs target proteins to the secretary or endocytic compartments of cells, is found in both EVs and cells. In many human diseases, e.g., cancer and cardiovascular diseases, sortilin expression levels are atypically increased. Although recent reports have noted that sortilin is regulated by multiple post-translational modifications, the precise mechanisms of sortilin trafficking has not been elucidated. Dimerization of sortilin regulates trafficking of the receptor to extracellular vesicles. Therefore, inhibiting dimerization of sortilin can act as a new therapeutic strategy for the treatment of EV-associated diseases, including vascular calcification and cancer, among others.

Accordingly, there is a need in the art for methods and compositions for inhibiting, decreasing, and preventing trafficking of sortilin to EVs.

### SUMMARY

This invention is based, in part, on the inventor's discovery inter alia that sortilin forms homodimers with an intermolecular disulfide bond at the cysteine 783 (Cys⁷⁸³) residue, and that formation of the disulfide bond leads to trafficking of sortilin to extracellular vesicles (EVs). Accordingly, in one aspect, provided herein is a method for inhibiting or reducing trafficking of sortilin to an extracellular vesicle (EV) from a cell. Generally, the method comprises inhibiting covalent intermolecular dimerization of sortilin in the cell. For example, by inhibiting formation of an intermolecular disulfide bond, e.g., an intermolecular disulfide bond at Cys⁷⁸³ of sortilin.

Without limitation, the cell can be any cell expressing sortilin. Exemplary cells include, but are not limited to, leukocytes, lymphocytes, macrophages, natural killer cells, dendritic cells, T cells, B cells, osteoblasts, osteoclasts, mesenchymal stem cells, endothelial cells, pancreatic cells (β, a, or y), pancreatic polypetptide (PP) cells, hepatocytes, adipose cells, and kidney glomerulus parietals, podocytes, or proximal tubule brush border cells. Further, the dimerization can be carried out by administering or contacting the cell with an agent that inhibits, reduces or prevents sortilin dimerization, for example by inhibiting, reducing or preventing formation of an intermolecular disulfide bond.

As discussed herein, inhibiting dimerization of sortilin can be used for treating EV-associated diseases. Thus, in another aspect, provided herein is a method of treating an extracellular vesicle associated disease in subject, comprising inhibiting covalent intermolecular dimerization of sortilin in a subject in need thereof. For example, inhibiting formation of an intermolecular disulfide bond, such as at Cys⁷⁸³ of sortilin. decrease, inhibit, reduce, and/or treat calcification in a subject in need thereof.

In another aspect, provided herein is a method for identifying a test agent that modulates dimerization of sortilin. The method comprises contacting a cell with a test agent, wherein the cell expresses a first sortilin polypeptide comprising a first label, and a second sortilin polypeptide comprising a second label. The labels can be same or different, and a distance or contact level between the two labels can be determined. Thus, after the cell is contacted with the test agent, a distance or contact level between the two labels is measured or determined. The distance or contact level is compared to a reference or distance or contact level. The reference or control can be a distance or contact level measured in a cell, i.e., the cell expressing a first sortilin polypeptide comprising a first label, and a second sortilin polypeptide comprising a second label, that is not contacted with the test compound. A change in distance or contact level relative to the control or reference level indicates the agent modulates dimerization of sortilin.

Any method known to one of skill in the art could be used for measuring or detecting the distance or contact level. For example, Fluorescence Resonance Energy Transfer (TR-FRET) can be used to measure the distance or contact level between the two labels. In some embodiments, the two lables can be a FRET donor and acceptor pair. In some embodiments, the ligands that can bind the two lables can be used, where the ligand that binds with the first label can comprise one of the FRET acceptor or donor and the ligand that bind the second label can comprise other of the FRET acceptor or donor.

Furthermore, provided herein is a method for preparing dimeric soluble sortilin. The method comprises expressing a sortilin polypeptide comprising a first label in an extracellular domain from a cell. The expressed sortilin polypeptide can be purified by affinity purification. For example, the affinity purification can be based on affinity for the first label. Generally, the purification is under conditions that reduce separation of sortilin dimers.

### BRIEF DESCRIPTION OF THE DRAWINGS

FIG. 1A-D shows that sortilin forms homodimers on the cell surface of HEK293 cells. FIG. 1A shows schematic of FLAG-sortilin and 6×His-sortilin. FLAG tag and 6×His tag were inserted following propeptide and 3 amino acids (Ser78-Ala79-Pro80) in sortilin. SP, signal peptide; PP, propeptide; TMD, transmembrane domain; ICD, intracellular domain. FIG. 1B shows overexpression of FLAG-sortilin and 6×His-sortilin in HEK293 cells was validated in western blotting. FIG. 1C-D shows detection of binding of FLAG-sortilin and 6×His-sortilin on the cell surface of HEK293 in TR-FRET assay (FIG. 1C) and HTRF assay (FIG. 1D). Change of FRET signal by expression of 6×His-sortilin was indicated as % change (mean±S.E., 3 independent experiments). t-test, *, p<0.05; **, p<0.01.

FIG. 2A-G shows sortilin forms homodimers in the extracellular and intracellular domains with intermolecular disulfide bonds in HEK293 cells. FIG. 2A shows a schematic of FLAG-sortilin full-length (Full), ECD+TMD and ICD+TMD. FIG. 2B-C shows protein expression of FLAG-sortilin Full, ECD+TMD and ICD+TMD was validated in reducing (FIG. 2B) and non-reducing (FIG. 2C) western blotting using anti-FLAG antibody. FLAG-sortilin Full and ECD+TMD forms homodimers and multimers. Empty vector was used as control. FIG. 2D shows HEK293 cells transiently overexpressing FLAG-sortilin Full or ECD+TMD were treated with cross-linker, and the cell lysates were used for reducing western blotting with anti-FLAG antibody, showing dimerization of sortilin Full and ECD+TMD (n=3). FIG. 2E shows HEK293 cells stably overexpressing FLAG-sortilin ICD+TMD (FLAG-sortilin ICD+TMD HEK293 cells) were incubated with DMSO (Control), MG-132 (MG) 20 μmol/L or chloroquine (Chlo) 10 μmol/L for 7 hours, and then reducing western blotting was performed using anti-sortilin antibody. MG-132 increased the protein expression of FLAG-sortilin ICD+TMD, but chloroquine did not (n=3). FIG. 2F shows FLAG-sortilin ICD+TMD HEK293 cells were incubated with MG-132 (2-20 μmol/L) for 7 or 24 hours. MG-132 increased FLAG-sortilin ICD+TMD in a time and concentration-dependent manner. FIG. 2G shows following 16-hour incubation of HEK293 cells (Control) or FLAG-sortilin ICD+TMD HEK293 cells (ICD+TMD) with MG-132 (5 μrnol/L) and immunoprecipitation with anti-FLAG antibody, non-reducing western blotting showed dimerization of sortilin ICD+TMD using anti-sortilin antibody. Monomers, homodimers and multimers are abbreviated as MO, D and MU, respectively. IB, immunoblotting.

FIG. 3A-D shows the transmembrane domain of sortilin forms homodimers via noncovalent interaction. FIG. 3A-D shows 6×His-sortilin Full, ECD+TMD, and ICD+TMD were transiently overexpressed in HEK293 cells with stably overexpressed FLAG-sortilin Full (FIG. 3A-B) and ECD+TMD (FIG. 3C-D), respectively. Immunoprecipitation with anti-FLAG M2 antibody was performed using the cell lysates. Western blotting was carried out using whole cell lysates (FIG. 3A, C) and immunoprecipitants (FIG. 3B, D). 6×His-sortilin Full, ECD+TMD and ICD+TMD were co-precipitated with FLAG-sortilin Full or ECD+TMD (B, D) (n=2). IB, immunoblotting.

FIG. 4A-D shows that substituting the transmembrane domain of sortilin with the corresponding domain of CD43 does not decrease the dimeric form of sortilin. FIG. 4A shows a schematic of FLAG-sortilin CD43-TMD. Transmembrane domain of sortilin was replaced with that of CD43. FIG. 4B shows FLAG-sortilin WT and FLAG-sortilin CD43-TMD were transiently overexpressed in HEK293 cells, and non-reducing Western blotting was carried out using cell lysate with anti-FLAG antibody (n=3). Monomers, homodimers, and multimers are abbreviated as MO, D, and MU, respectively. FIG. 4C-D shows 6×His-sortilin WT or 6×His-sortilin CD43-TMD was transiently overexpressed in HEK293 cells stably overexpressing FLAG-sortilin, and immunoprecipitation was performed using anti-FLAG M2 antibody. Western blotting was carried out using whole cell lysates (FIG. 4C) and immunoprecipitants (FIG. 4D). 6×His-sortilin CD43-TMD coprecipitated with FLAG-sortilin as well as 6×His-sortilin WT. Arrows, sortilin wildtype or sortilin CD43-TMD (n=3). FIG. 4 E shows that in FLAG-sortilin HEK293 cells or HEK293 cells, 6×His-sortilin CD43-TMD was overexpressed. The cells were subjected to TR-FRET assay. Change of FRET signal by expression of 6×His-sortilin WT or CD43-TMD is indicated by percent change (mean S.D., n=4, one independent experiment). Error bars represent S.D. *P<0.05; **P<0.01 by t test. FIG. 4F-G shows that in FLAG-sortilin HEK293 cells, 6×His-sortilin WT or 6×His-sortilin CD43-TMD were overexpressed. The cell lysates were subjected to non-reducing Western blotting with anti-FLAG antibody (FIG. 4F) and anti-6×His antibody (FIG. 4G), demonstrating that substituting the transmembrane domain of sortilin with that of CD43 did not decrease dimerization (n=3). IB, immunoblotting.

FIG. 5A-F shows that mutation of Cys⁷⁸³ abolished dimerization of sortilin. FIG. 5A shows a schematic of FLAG-sortilin wild-type (WT) and C783A, and 6×His-sortilin ICD TMD WT and C783A. Cysteine 783 was replaced by Alanine. SP, signal peptide; PP, propeptide. FIG. 5B shows expression vector of 6×His-sortilin 10CC TMD was transfected in HEK293 cells. Dimerization of 6×His-sortilin 10CC TMD was detected in non-reducing Western blotting with anti-6×His antibody (n=3). FIG. 5C-D shows C783A decreased homodimers of sortilin in the cells (FIG. 5C) and extracellular vesicles (FIG. 5D) of HEK293 cells in the non-reducing western blotting (n=3). FIG. 5D-E shows C783A decreased sortilin homodimers of low molecular weight in the cells (FIG. 5D) and extracellular vesicles (FIG. 5E) of HEK293 cells in non-reducing Western blotting (n=3). FIG. 5F-G shows 24-h incubation with 2-FPA, an inhibitor of palmitoylation, increased sortilin homodimers of low molecular weight in HEK293 cells stably overexpressing FLAG-sortilin (FIG. 5F) and their extracellular vesicles (FIG. 5G) (n=3). Monomers and homodimers of high and low molecular weight are abbreviated as MO, D(HMW), and D(LMW), respectively. IB, immunoblotting.

FIG. 6A-E shows that sortilin 5316E and without propeptide (wp) increases dimerization in HEK293 cells, and the addition of propeptide decreases dimerization in the extracellular vesicles of FLAG-sortilin HEK293 cells. FIG. 6A shows a schematic of FLAG-sortilin WT, S316E and without propeptide (wp). Serine316 was replaced by glutamic acid in sortilin (S316E). Propeptide was removed in FLAG-sortilin wp. SP, signal peptide; PP, propeptide FIG. 6B shows 5316E increased dimerization of sortilin in HEK293 cells (n=3). FIG. 6C shows that removal of propeptide increased dimerization of sortilin in HEK293 cells (n=3). FIG. 6D-E shows addition of propeptide (100 nmol/L) decreased dimerization of sortilin in the extracellular vesicles of FLAG-sortilin HEK293 cells (FIG. 6E), whereas a decrease in the cells was not observed (FIG. 6D) (n=2). Monomers and homodimers of high and low molecular weight are abbreviated as MO, D(HMW), D(LMW), respectively. Vex, vector; IB, immunoblotting.

FIG. 7A-E shows that soluble sortilin forms homodimers. FIG. 7A-B shows orientation of sortilin on the EV membrane was determined using EVs secreted from FLAG-sortilin HEK293 cells (FIG. 7A) and sortilin-3×FLAG HEK293 cells (FIG. 7B). EVs or their lysates were subjected to immunoprecipitation with anti-FLAG M2 antibody, and FLAG-sortilin (FIG. 7A) or sortilin-3×FLAG (FIG. 7B) was detected in western blotting with anti-FLAG antibody (n=3). FIG. 7C-D shows soluble sortilin secreted by HEK293 cells overexpressing FLAG-sortilin Full and FLAG-sortilin ECD+TMD was detected in non-reducing (FIG. 7C) and reducing western blotting (FIG. 7D), showing that they were homodimers and monomers, respectively (n=2). FIG. 7E shows soluble sortilin secreted by HEK293 cells overexpressing FLAG-sortilin Full and FLAG-sortilin ECD+TMD was purified and detected in non-reducing western blotting. IB, immunoblotting.

FIG. 8 shows a schematic figure showing involvement of dimerization for trafficking of sortilin and the regulation by propeptide. Dissociation of propeptide from sortilin promoted dimerization. Dimerization with an intermolecular disulfide bond at Cys⁷⁸³ facilitated transport of dimerized sortilin to the extracellular vesicles. Cys⁷⁸³ is associated with dimerization and palmitoylation. Palmitoylated sortilin is transported back to the Golgi apparatus. (1) Propeptide is cleaved from sortilin. (2) Propeptide binds to sortilin at different location. Then, sortilin is transported through the Golgi apparatus. (3) Sortilin forms homodimers with intermolecular disulfide bonds at 10CC domain and Cys⁷⁸³. (4) Sortilin is incorporated to endosome by endocytosis. (5) Palmitoylated sortilin monomer is transported back to Golgi by interaction with retromer. (6) Sortilin homodimer is secreted by extracellular vesicles (microvesicles and/or exosomes). (4) Sortilin homodimer is shedded and secreted as soluble sortilin.

FIG. 9 shows that in FLAG-sortilin HEK293 cells or HEK293 cells, 6×His-sortilin, CD43-TMD was overexpressed. The cells were subjected to TR-FRET assay. Change of FRET signal by expression of 6×His-sortilin wild-type (WT) or CD43-TMD was indicated as change (mean±S.E., n=4, 1 independent experiment). t-test, *P<0.05; **, p<0.01.

FIG. 10A-B shows the expression vector of 6×His-sortilin 10CC+TMD was constructed (FIG. 10A) and transfected in HEK293 cells. Dimerization of 6×His-sortilin 10CC+TMD was detected in the non-reducing western blotting with anti-6×His antibody (FIG. 10B) (n=3).

FIG. 11A-B shows HCASMCs expressing His-wild type sortilin were infected with LacZ, Flag-wild type sortilin, Flag-C783A mutant sortilin and Flag-S316E mutant sortilin. FIG. 11A shows HCASMCs were lysed 3 days after infection. Sortilin expression was detected by the non-reducing or reducing (cleavage disulfide bounds) western blotting in input sample. FIG. 11B shows the immunoprecipitated sample. Dimer form was decreased in C783A mutant and multimer form was increased in S316E mutant. MO=Monomer, D=dimer and MU=multimer. N=3 donors.

FIG. 12 shows HCASMCs expressing His-wild type sortilin were infected with LacZ, Flag-wild type sortilin, Flag-C783A mutant sortilin and Flag-S316E mutant sortilin. HCASMCs-derived EVs were isolated from culture medium 3 days after infection. Sortilin expression was detected by the non-reducing or reducing western blotting. Dimer form was decreased in C783A mutant and increased in S316E mutant. These suggest that the trafficking of sortilin to EVs is decreased in C783A mutant and increased in S316E mutant. MO=Monomer, D=dimer and MU=multimer. N=3 donors.

### DETAILED DESCRIPTION

As used herein, the term “sortilin” or “sortilin 1” refers to a type I membrane glycoprotein in the vacuolar protein sorting 10 protein (Vps10p) family of sorting receptors that is found within or on the surface of extracellular vesicles and within cells. The protein sequence of sortilin is provided herein as SEQ ID NO: 1. Sortilin is encoded by the SORT1 gene (NCBI Reference Sequence: NG_028280.1). The human SORT1 sequence is provided herein as SEQ ID NO: 3. SORT1 or sortilin can refer to human SORT1, including naturally occurring variants, molecules, and alleles thereof. SORT1 refers to the mammalian SORT1 of, e.g., mouse, rat, rabbit, dog, cat, cow, horse, pig, and the like.

In humans, sortilin is expressed in a wide range of tissues including the brain, heart, skeletal muscle, adrenal gland, thyroid, B-lymphocytes, adipocytes, and the spinal cord. Sortilin is involved in the transport of intracellular proteins between the trans-Golgi network, endosome, lysosome, secretory granules, and between cellular plasma membranes. Exemplary proteins transported by sortilin include but are not limited to tissue non-specific alkaline phosphatase (TNAP), Caveolin-1, beta-secretase 1 (BACE 1), epidermal growth factor receptor (EGFR), tropomyosin receptor kinase B (TrkB), apolipprotein E (APOE), progranulin, amyloid precursor protein (APP), glucose transporter type 4 (GLUT4), low-density lipoprotein (LDL)-cholesterol, very low-density lipoprotein (VLDL), and proprotein convertase subtilisin/kexin type 9 (PCSK9).

In embodiments of the various aspects described herein, intermolecular dimerization of sortilin can be by administering or contacting a cell with an agent that inhibits, prevents or reduces intermolecular dimerization. For example, the agent inhibits, prevents or reduces formation of intermolecular disulfide bond(s) in a sortilin polypeptide.

In some embodiments of the various aspects described herein, intermolecular dimerization of sortilin is inhibited by inhibiting formation of an intermolecular disulfide formation at Cys783 of SEQ ID NO: 1.

The agent capable of inhibiting dimerization of sortilin is also referred to as an “inhibitor of sortilin” or “inhibitor” herein. Without limitations, the inhibitor, can be selected from the group consisting of a small molecule, nucleic acid, polypeptide, drug, ion, small organic or inorganic molecules, saccharines, oligosaccharides, polysaccharides, biological macromolecules, e.g., peptides, proteins, and peptide analogs and derivatives; peptidomimetics; antibodies and antigen binding fragments thereof, nucleic acids; nucleic acid analogs and derivatives; an extract made from biological materials such as bacteria, plants, fungi, or animal cells; animal tissues; naturally occurring or synthetic compositions; and any combinations thereof.

In some embodiments of the various aspects disclosed herein, the inhibitor is a peptide. As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

In some embodiments of the various aspects disclosed herein, the inhibitor is a sortilin-derived propeptide. As used herein, the term “propetide” refers to a peptide that is a precursor to a peptide or protein that comprises a post-translational modification such as a cleavage site to become biologically active. In some embodiments, the propeptide comprises the amino acid sequence of SEQ ID NO: 2. In one embodiment, the propeptide comprises at least on amino acid substitution or a conservative substitution of SEQ ID NO: 2. In another embodiment, the sortilin-derived propeptide is amidated, acetylated, cyclized, phosphorylated, glycosylated, nitrosylated, methylated, lipidated, or PEGylated. In another embodiment, the peptide comprises at least one D amino acid, beta amino acid or modified peptide linkage.

The term “conservative substitution,” when describing a polypeptide, refers to a change in the amino acid composition of the polypeptide that does not substantially alter the polypeptide's activity, fore examples, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine. “Conservative amino acid substitutions” result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not substantially alter activity. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In addition, individual substitutions, deletions or additions that alter, add or delete a single amino acid or a small percentage of amino acids in an encoded sequence are also “conservative substitutions.” Insertions or deletions are typically in the range of about 1 to 5 amino acids.

In some embodiments of the various aspects disclosed herein, the inhibitor is a small molecule. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases, it is preferred that a small molecule have a molecular weight equal to or less than 700 Daltons.

In some embodiments of the various aspects disclosed herein, the inhibitor is a nucleic acid molecule or an analog or derivate thereof. As used herein, the term “nucleic acid” or “oligonucleotide” or grammatical equivalents herein means at least two nucleotides, including analogs or derivatives thereof that are covalently linked together. Exemplary oligonucleotides include, but are not limited to, single-stranded and double-stranded siRNAs and other RNA interference reagents (RNAi agents or iRNA agents), shRNA (short hairpin RNAs), antisense oligonucleotides, aptamers, ribozymes, and microRNAs (miRNAs). The nucleic acids can be single stranded or double stranded. The nucleic acid can be DNA, RNA or a hybrid, where the nucleic acid contains any combination of deoxyribo- and ribonucleotides, and any combination of uracil, adenine, thymine, cytosine and guanine. The nucleic acids can comprise one or more backbone modifications, e.g., phosphoramidate (Beaucage et al., Tetrahedron 49(10):1925 (1993) and references therein; Letsinger, J. Org. Chem. 35:3800 (1970)), phosphorothioate, phosphorodithioate, O-methylphophoroamidite linkages (see Eckstein, Oligonucleotides and Analogues: A Practical Approach, Oxford University Press), or peptide nucleic acid linkages (see Egholm, J. Am. Chem. Soc. 114:1895 (1992); Meier et al., Chem. Int. Ed. Engl. 31:1008 (1992); and Nielsen, Nature, 365:566 (1993), content of all of which is herein incorporated by reference. The nucleic acids can also include modifications to nucleobase and/or sugar moieties of nucleotides. Exemplary sugar modifications at the sugar moiety include replacement of 2′-OH with halogens (e.g., fluoro), O-methyl, O-methoxyethyl, NH2, SH and S-methyl. In some embodiments, the nucleic acid is a peptide nucleic acid (PNA). Without wishing to be bound by a theory, nucleic acid inhibitors can decrease, inhibit, or reduce the expression or amount of the nucleic acid encoding a component of the complex. Computational and experimental methods, including high throughput screening assays, for producing nucleic acid inhibitors, e.g., antisense oligonucleotides, siRNAs, ribozymes, aptamers, and the like, targeted to any target sequence are known in the art and available to one of skill in the art.

The term “oligonucleotide” as used herein refers to a short nucleic acid polymer, typically with twenty or fewer bases.

The term “shRNA” as used herein refers to short hairpin RNA which functions as RNAi and/or siRNA species but differs in that shRNA species are double stranded hairpin-like structure for increased stability. The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi can result in the destruction of specific mRNA, or prevents the processing or translation of RNA, such as mRNA.

In some embodiments, the inhibitor is short interfering RNA (siRNA). The term “short interfering RNA” (siRNA), also referred to herein as “small interfering RNA” is defined as an agent which functions to inhibit expression of a target gene, e.g., by RNAi. A siRNA can be chemically synthesized, it can be produced by in vitro transcription, or it can be produced within a host cell. siRNA molecules can also be generated by cleavage of double stranded RNA, where one strand is identical to the message to be inactivated. The term “siRNA” refers to small inhibitory RNA duplexes that induce the RNA interference (RNAi) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region.

In some embodiments, the inhibitor is an antisense oligonucleotide or siRNA molecule comprising a part of (e.g., 10-50, 12-40, 15-30, 16-25, or 18-22 consecutive nucleotides) of the antisense sequence of a nucleic acid encoding sortilin, e.g. SORT 1. Nucleic acid sequence of human sortilin proteins can be accessed by NCBI Reference Gene ID: 6272, NCBI Reference Sequence: NG 028280.1, and SEQ ID NO: 3. In some embodiments, the nucleic acid encoding sortilin is SORT mRNA.

In some embodiments, the inhibitor is an aptamer. As used herein, the term “aptamer” means a single-stranded, partially single-stranded, partially double-stranded or double-stranded nucleotide sequence capable of specifically recognizing a selected non-oligonucleotide molecule or group of molecules. Accordingly, aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9(1999), and Hermann and Patel, Science 287:820-5 (2000). Aptamers can be RNA or DNA based. Methods for selecting aptamers for binding to a molecule are widely known in the art and easily accessible to one of ordinary skill in the art. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer can be prepared by any known method, including synthetic, recombinant, and purification methods, and can be used alone or in combination with other aptamers specific for the same target. Further, as described more fully herein, the term “aptamer” specifically includes “secondary aptamers” containing a consensus sequence derived from comparing two or more known aptamers to a given target. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. In some embodiments, the aptamer recognizes the non-oligonucleotide molecule or group of molecules by a mechanism other than Watson-Crick base pairing or triplex formation.

As used herein, the term “polysaccharide” refers to macromolecular carbohydrates whose molecule consists of a large number of monosaccharide molecules which are joined to one another by glycosidic linkage. The term polysaccharide is also intended to embrace an oligosaccharide. The polysaccharide can be homopolysaccharides or heteropolysaccharides. Whereas the homopolysaccharides contain only one kind of unit, the heteropolysaccharides consist of monomer units of different kinds.

In some embodiments, the inhibitor is an antibody or a fragment thereof. The terms “antibody” and “antibodies” include polyclonal antibodies, monoclonal antibodies, humanized or chimeric antibodies, single chain Fv antibody fragments, Fab fragments, and F(ab)2 fragments. Antibodies having specific binding affinity for sortilin 1 can be produced through standard methods. Alternatively, antibodies can be commercially available, for example, from R&D Systems, Inc., Minneapolis, Minn.

As used herein, the terms “antibody” and “antibodies” include intact antibody, or a binding fragment thereof that competes with the intact antibody for specific binding and includes chimeric, humanized, fully human, and bispecific antibodies. In some embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)2, Fv, and single-chain antibodies.

Unless it is specifically noted, as used herein a “fragment thereof” in reference to an antibody refers to an immunespecific fragment, i.e., an antigen-specific or binding fragment.

Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler, G. et al., Nature, 1975, 256:495, the human B-cell hybridoma technique (Kosbor et al., Immunology Today, 1983, 4:72; Cole et al., Proc. Natl. Acad. Sci. USA, 1983, 80:2026), and the EBV-hybridoma technique (Cole et al., Monoclonal Antibodies and Cancer Therapy, Alan R. Liss, Inc., 1983, pp. 77-96). Such antibodies can be of any immunoglobulin class including IgG, IgM, IgE, IgA, IgD, and any subclass thereof. The hybridoma producing the monoclonal antibodies of the invention can be cultivated in vitro or in vivo.

Polyclonal antibodies are heterogeneous populations of antibody molecules that are specific for a particular antigen, which are contained in the sera of the immunized animals. Polyclonal antibodies are produced using well-known methods. A chimeric antibody is a molecule in which different portions are derived from different animal species, such as those having a variable region derived from a murine monoclonal antibody and a human immunoglobulin constant region. Chimeric antibodies can be produced through standard techniques. Antibody fragments that have specific binding affinity for a component of the complex can be generated by known techniques. For example, such fragments include, but are not limited to, F(ab′)2 fragments that can be produced by pepsin digestion of the antibody molecule, and Fab fragments that can be generated by reducing the disulfide bridges of F(ab′)2 fragments. Alternatively, Fab expression libraries can be constructed. See, for example, Huse et al., 1989, Science, 246: 1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

In some embodiments, the antibody or antigen-binding fragment thereof is murine. In some embodiments, the antibody or antigen-binding fragment thereof is from rabbit. In some embodiments, the antibody or antigen-binding fragment thereof is from rat. In other embodiments, the antibody or antigen binding fragment thereof is human. In some embodiments the antibody or antigen-binding fragment thereof is recombinant, engineered, humanized and/or chimeric.

In some embodiments, an antibody, or antigen binding fragment, variant, or derivative thereof for use in the methods of the invention binds specifically to at least one epitope of target molecule (e.g., sortilin), i.e., binds to such an epitope more readily than it would bind to an unrelated, or random epitope; binds preferentially to at least one epitope of sortilin, i.e., binds to such an epitope more readily than it would bind to a related, similar, homologous, or analogous epitope; competitively inhibits binding of a reference antibody which itself binds specifically or preferentially to an epitope of the target molecule (e.g., sortilin); or binds to at least one epitope of the target molecule (e.g., sortilin) with an affinity characterized by a dissociation constant Kd of about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁸ M, about 10⁻⁸ M, about 5×10⁹ M, about 10⁻⁹ M, about 5×10¹⁰ M, about 10¹⁰ M, about 5×10⁻¹¹ M, about 10⁻¹¹ M, about 5×10′² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M.

In some embodiments, the antibody or fragment thereof preferentially binds to a human sortilin polypeptide or fragment thereof, relative to a murine sortilin polypeptide or fragment thereof.

As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” can include, for example, from 0.05 M to 0.005 M.

In some embodiments, an antibody, or antigen binding fragment, variant, or derivative thereof for use in the methods provided herein binds sortilin polypeptides or fragments or variants thereof with an off rate (k(off)) of less than or equal to about 5×10⁻² sec-1, about 10⁻² sec-1, about 5×10⁻³ sec-1, about 10⁻³ sec-1, about 5×10⁻⁴ sec-1, about 10⁻⁴ sec-1, about 5×10⁻⁴ sec-1, about 10⁻⁴ sec-1, about 5×10⁻⁵ sec-1, about 10⁻⁵ sec-1, about 5×10⁻⁶ sec-1, about 10⁻⁶ sec-1, about 5×10⁻⁷ sec-1, or about 10⁻⁷ sec-1.

In other embodiments, an antibody, or antigen-binding fragment, variant, or derivative thereof for use in the methods provided herein binds sortilin polypeptides or fragments or variants thereof with an on rate (k(on)) of greater than or equal to about 10³ M-1 sec-1, about 5×10³ M-1 sec-1, 10⁴ M-1 sec-1, about 5×10⁴ M-1 sec-1, 10⁵ M-1 sec-1, about 5×10⁵ M-1 sec-1, 10⁶ M-1 sec-1, about 5×10⁶ M-1 sec-1, 10⁷ M-1 sec-1, or about 5×10⁷ M-1 sec-1. The binding affinity and dissociation rate of an antibody for use in the methods provided herein can be determined by any method known in the art. For example, the binding affinity can be measured by competitive ELISAs, RIAs, BIACORE™, or KINEXA™ technology. The dissociation rate also can be measured by BIACORE™ or KINEXA™ technology.

In some embodiments, an antibody or an antigen-binding fragment for use in the methods provided herein modulates the binding of a second molecule to sortilin. In some embodiments, the modulation is enhancement of the binding of the second molecule to sortilin. In some embodiments, the modulation is inhibition of the binding of the second molecule to sortilin. The IC50 of such inhibition can be measured by any method known in the art, e.g., by ELISA, RIA, or Functional Antagonism. In some embodiments, the IC50 is between 0.1 and 500 nM. In some embodiments, the IC50 is between 10 and 400 nM. In yet other embodiments, the antibody or portion thereof has an IC50 of between 60 nM and 400 nM.

Antibodies for use in the methods of the invention can be generated by immunization of a suitable host (e.g., vertebrates, including humans, mice, rats, sheep, goats, pigs, cattle, horses, reptiles, fishes, amphibians, and in eggs of birds, reptiles and fish). Such antibodies can be polyclonal or monoclonal. In some embodiments, the host is immunized with an immunogenic sortilin. In other embodiments, the host is immunized with sortilin associated with a cell membrane of an intact or disrupted cell and antibodies for use in the methods of the invention are identified by binding to sortilin.

In some embodiments, the sortilin antigen is administered with an adjuvant to stimulate the immune response. Adjuvants often need to be administered in addition to antigen in order to elicit an immune response to the antigen. These adjuvants are usually insoluble or nondegradable substances that promote nonspecific inflammation, with recruitment of mononuclear phagocytes at the site of immunization. Examples of adjuvants include, but are not limited to, Freund's adjuvant, RIBI (muramyl dipeptides), ISCOM (immunostimulating complexes) or fragments thereof.

For a review of methods for making antibodies, see, e.g., Harlow and Lane, Antibodies, A Laboratory Manual (1988); Yelton, D. E. et al., Ann. Rev. of Biochem. 50:657-80. (1981); and Ausubel et al., Current Protocols in Molecular Biology (New York: John Wiley & Sons) (1989). Determination of immunoreactivity with an immunogenic sortilin polypeptide can be made by any of several methods well known in the art, including, e.g., immunoblot assay and ELISA.

Anti-sortilin antibodies for use in the methods provided herein can be of any isotype. An antibody of any desired isotype can be produced by class switching. For class switching, nucleic acids encoding V_{L} or V_{H}, that do not include any nucleotide sequences encoding C_{L} or C_{H}, are isolated using methods well known in the art. The nucleic acids encoding V_{L} or V_{H} are then operatively linked to a nucleotide sequence encoding a C_{L} or C_{H} from a desired class of immunoglobulin molecule. This can be achieved using a vector or nucleic acid that comprises a C_{L} or C_{H} chain, as described above. For example, an anti-sortilin antibody for use in the methods provided herein that was originally IgM can be class switched to an IgG. Further, the class switching can be used to convert one IgG subclass to another, e.g., from IgG1 to IgG2.

The class and subclass of anti-sortilin antibodies can be determined by any method known in the art. In general, the class and subclass of an antibody can be determined using antibodies that are specific for a particular class and subclass of antibody. Such antibodies are available commercially. The class and subclass can be determined by ELISA, Western Blot, as well as other techniques. Alternatively, the class and subclass can be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

In certain embodiments, both the variable and constant regions of sortilin antibodies or immunospecific fragments thereof for use in the treatment methods disclosed herein are fully human. Fully human antibodies can be made using techniques that are known in the art and as provided herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems, as described in more detail elsewhere herein.

Antibodies or fragments thereof for use in the treatment methods disclosed herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, or derivatization by known protecting/blocking groups, proteolytic cleavage, linkage to a cellular ligand or other protein. Any of numerous chemical modifications can be carried out by known techniques, including, but not limited to, specific chemical cleavage, acetylation, formylation, etc. Additionally, the derivative can contain one or more non-classical amino acids.

In some embodiments, antibody or fragment thereof for use in the methods disclosed herein will not elicit a deleterious immune response in the mammal to be treated, e.g., in a human. In one embodiment, the antibodies or fragments thereof for use in the methods disclosed herein can be modified to reduce their immunogenicity using art-recognized techniques. For example, antibodies can be humanized, primatized, deimmunized, or chimeric antibodies can be made. These types of antibodies are derived from a nonhuman antibody, typically a murine or primate antibody, that retains or substantially retains the antigen-binding properties of the parent antibody, but which is less immunogenic in humans. This can be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. USA 81:6851-6855 (1984); Morrison et al., Adv. Immunol. 44:65-92 (1988); Verhoeyen et al., Science 239:15341536 (1988); Padlan, Molec. Immun. 28:489-498 (1991); Padlan, Molec. Immun. 31:169-217 (1994), and U.S. Pat. Nos. 5,585,089; 5,693,761; 5,693,762; and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, VH and VL sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., anti-sortilin antibodies or immunospecific fragments thereof for use in the methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

Completely human antibodies are particularly desirable for therapeutic treatment of human patients. Human antibodies can be made by a variety of methods known in the art including phage display methods using antibody libraries derived from human immunoglobulin sequences. See, for example, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered nonfunctional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety.

Completely human antibodies which recognize a selected epitope can be generated using a technique referred to as “guided selection.” In this approach a selected nonhuman monoclonal antibody, e.g., a mouse antibody, is used to guide the selection of a completely human antibody recognizing the same epitope. (Jespers et al., Bio/Technology 12:899-903 (1988)). See also, U.S. Pat. No. 5,565,332. [0162] In another embodiment, DNA encoding desired monoclonal antibodies can be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA can be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which can be synthetic as provided herein) can be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody can be grown up in relatively large quantities to provide clinical and commercial supplies of the immunoglobulin.

Anti-sortilin antibodies can be purchased from Abcam (Cambridge, MA, USA) or R&D Systems (Minneapolis, MN, USA). Further, methods for identification and design of ligands capable of binding specifically to Sortilin are described, for example, in US Patent Application Publication No. 2011/1060439, content of which is incorporated herein by reference.

For inhibiting the dimerization of sortilin, the agent, i.e., the inhibitor, can be administered to the cell or contacted with the cell. Without limitations, the agent can be administered to the cell or contacted with the cell in a cell culture e.g., in vitro or ex vivo, or the agent can be administrated to a subject, e.g., in vivo. In some embodiments, the agent can be administrated to a subject to inhibit intermolecular dimerization of sortilin.

The term “contacting” or “contact” as used herein in connection with contacting a cell includes subjecting the cells to an appropriate culture media, which comprises the indicated inhibitor. Where the cell is in vivo, “contacting” or “contact” includes administering the inhibitor in a pharmaceutical composition to a subject via an appropriate administration route such that the inhibitor contacts the cell in vivo.

The term “ex vivo” refers to cells which are removed from a living organism and cultured outside the organism (e.g., in a test tube). If ex vivo methods are employed, cells or tissues can be removed and maintained outside the body according to standard protocols well known in the art. The compositions can be introduced into the cells via methods known or available to one of skill in the art. For example, the cells can be kept in a culture and inhibitor can be added to the culture media. The treated cells can then be infused (e.g., in a pharmaceutically acceptable carrier) or homotopically transplanted back into the subject per standard methods for the cell or tissue type. Standard methods are known for transplantation or infusion of various cells into a subject.

As provided herein, the inhibitor can be administered in a pharmaceutically acceptable carrier and can be delivered to the subject's cells in vivo and/or ex vivo by a variety of mechanisms well known in the art.

Generally, any amount of the agent can be contacted with the cell. In some embodiments, the agent is contacted at a concentration in the range of from about 0.1 nM to about 1000 mM. Preferably the compound is contacted in the range of from about 0.1 μM to about 10 μM. Additionally, the compound can be contacted with the cell for a sufficient time to allow the compound to be taken up by the cell and interact with its target.

As disclosed herein, the cell can be contacted with the inhibitor in a cell culture e.g., in vitro or ex vivo, or the inhibitor can be administrated to a subject, e.g., in vivo. In some embodiments, the inhibitor can be administrated to a subject to decrease, inhibit, reduce, and/or treat calcification. In some embodiments, the cell is a leukocyte, lymphocyte, macrophage, natural killer cell, dendritic cell, T cell, or B cell. In some embodiments, the cell is an interstitial valvular cell. In some embodiments, the cell is an osteoblast. In some embodiments, the cell is an osteoclast. In some embodiments, the cell is a mesenchymal stem cell. In some embodiments, the cell is an endothelial cell. In some embodiments, the cell is a pancreatic β, a, y, or pancreatic polypetptide (PP) cell. In some embodiments the cell is a hepatocyte. In some embodiments, the cell is a kidney glomerulus parietal, podocyte, or proximal tubule brush border cell. In some embodiments, the cell is an adipose cell. In some embodiments, the cell is a macrophage. In some embodiments, the cell is a monocyte. In some embodiments, the cell is a dendritic cell. In some embodiments, the cell is a lymphocyte. In some embodiments, the cell is a vascular smooth muscle cell.

In some embodiments, the inhibitor can be administered to a subject in conjunction with surgical and non-surgical treatments. In some embodiments, the methods disclosed herein can be practiced in injunction with dialysis.

As disclosed herein, inhibiting intermolecular dimerization of sortilin can be beneficial for treating EV-associated diseases. Accordingly, in another aspect, provided herein is a method for treating an EV-associated disease in a subject. Generally, the method comprises inhibiting covalent intermolecular dimerization of sortilin subject in need thereof. For example, inhibiting formation of an intermolecular disulfide bond at the Cys⁷⁸³ of sortilin. In some embodiments, the method comprises administering an agent to a subject in need thereof, wherein the agent inhibits intermolecular dimerization. In some embodiments, the agent that is administered to the subject is a sortilin propeptide.

As used herein, an “extracellular vesicle-associated disease” or “EV-associated disease” refers to a disease that has been diagnosed, suspected, or has not yet developed in a subject that is caused by aberrant extracellular vesicle trafficking, calcification of extracellular vesicles, or mislocalization of extracellular vesicles that results in at least one symptom and/or characteristic of the disease (e.g. calcification, inflammation, vesicle accumulation, pain, fatigue, shortness of breath, etc). The EV-associated disease can be caused by trafficking of sortilin, a sortilin dimer, or multimer to an extracellular vesicle(s) from a cell. As provided herein, the EV-associated disease can have variable expression of monomers and dimers of soluble sortilin, and the monomer/dimer ratio in serum for these diseases can be used for the diagnosis, prevention, and treatment of the EV-associated disease.

Extracellular vesicles are known in the art to contribute to physiology and pathology of various diseases (See Krohn and Aikawa et al. Journal of Physiology, 2016). Furthermore, sortilin has been found to be elevated in serum levels of subjects with aortic calcification and genome wide association studies have shown that sortilin is associated with cardiovascular calcification (See Goettsch, JCI, 2016; and Goettsch et al. Arterioscler. Thromb. Vasc. Biol., 2018). Thus, the dimerization and trafficking of sortilin by extracellular vesicles is a characteristic of EV-associated diseases as provided herein.

Non-limiting examples of EV-associated diseases include but are not limited to calcific aortic valve disease, diabetes, systemic lupus erythematosus, ulcerative colitis, pulmonary fibrosis, nan-alcoholic fatty liver disease, osteoporosis, neurodegenerative disorders (e.g. Alzheimer's disease), scleroderma, atherosclerosis, myocardial infarction, hypercholesterolemia, cancer, rheumatoid arthritis, and obesity. In one embodiment, the diabetes is type 1 diabetes, type 2 diabetes, or maturity onset diabetes in the young. In another embodiment, the cancer is lung cancer or pancreatic cancer. In some embodiments, the EV-associated disease is acute kidney injury (AKI) or chronic kidney disease (CKD).

EV-associated diseases can be caused by extracellular vesicle-derived microcalficiations. These microcalcifications contribute to plaque instability (See also Goettsch et al. Circ Research, 2013). Identification of extracellular vesicle microcalcification, e.g., of the aortic valve, can be determined by structure illumination methods, zeta potential, confocal microscopy, Fourier-transform infrared spectroscopy (FTIR), X-ray spectroscopy, scanning electron microscopy (SEM), transmission electron microscopy (TEM), finite element analysis (FEA), or any other methods known in the art that can identify various stages of microcalcification (See also Hutechnson et al. Nature Materials 2016).

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects provided herein, the subject is a mammal, e.g., a primate, e.g., a human. The terms, “patient” and “subject” are used interchangeably herein.

In some embodiments, the subject is a mammal. The mammal can be a human, non-human primate, mouse, rat, dog, cat, horse, or cow, but are not limited to these examples. Mammals other than humans can be advantageously used as subjects that represent animal models of disorders.

The subject can be initially diagnosed by a licensed physician and/or authorized medical practitioner, and a regimen for prophylactic and/or therapeutic treatment via a method provided herein can be suggested, recommended or prescribed. Thus, in some embodiments, the method comprises selecting a subject for treatment for an EV-associated disease (e.g. vascular calcification).

Animal models that are reliable indicators of EV-associated diseases, that include but are not limited to human atherosclerosis, renal failure, hyperphosphatemia, diabetes, age-related vascular calcification and other conditions associated with vascular calcification are known in the art. For example, an experimental model of calcification of the vessel wall is described by Yamaguchi et al., Exp. Path. 25: 185-190, 1984, content of which is incorporated herein by reference in its entirety.

By “treatment or amelioration” is reversing, alleviating, ameliorating, inhibiting, slowing down or stopping the progression, aggravation or deterioration the progression or severity of a condition. In some embodiments, at least one symptom is alleviated by at least 20%, at least 30%, at least 40%, at least 50%, at least 60%, at least 70%, at least 80%, at least 90%, or at least 95% but not 100%, i.e. not a complete alleviation. In some embodiments, at least one symptom is completely alleviated.

The terms “therapeutic agents” or “agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent can be used which are capable of being released from the subject composition into adjacent tissues or fluids upon administration to a subject. Examples include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antiviral s, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, proteins, antibodies, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals.

As used herein, the terms “inhibiting,” “decreasing,” “preventing,” and “treating” in connection with an EV-associated disease, are intended to mean preventing, retarding, or reversing formation, translocation of extracellular vesicle and/or the growth or deposition of extracellular matrix hydroxyapatite crystal deposits. Without limitations, the improvement in disorder severity includes the reversal of the disease symptoms, as well as slowing down the progression of the disease.

For example, inhibiting formation of an intermolecular disulfide bond at Cys783 of SEQ ID NO: 1. The Cys783 is indicated within the sequence as bolded and underlined (e.g. C). The dimerization of sortilin can be inhibited by an agent, for example, a sortilin-derived propeptide.

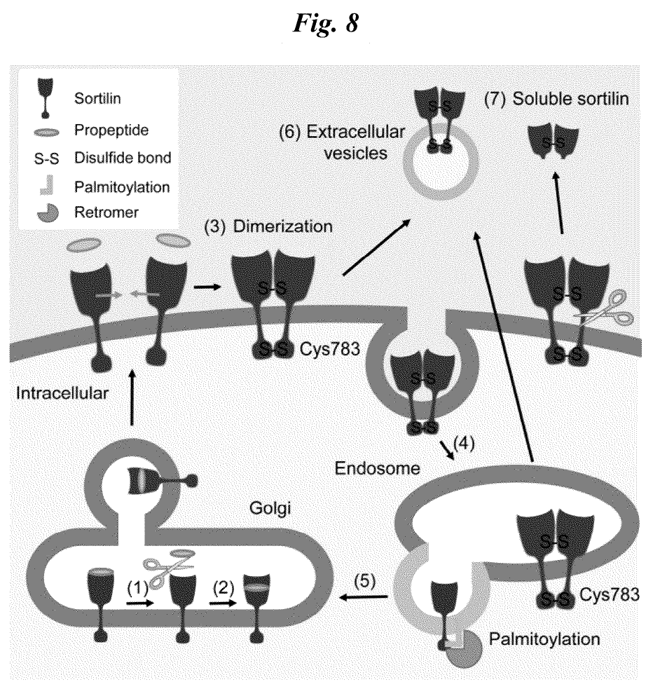

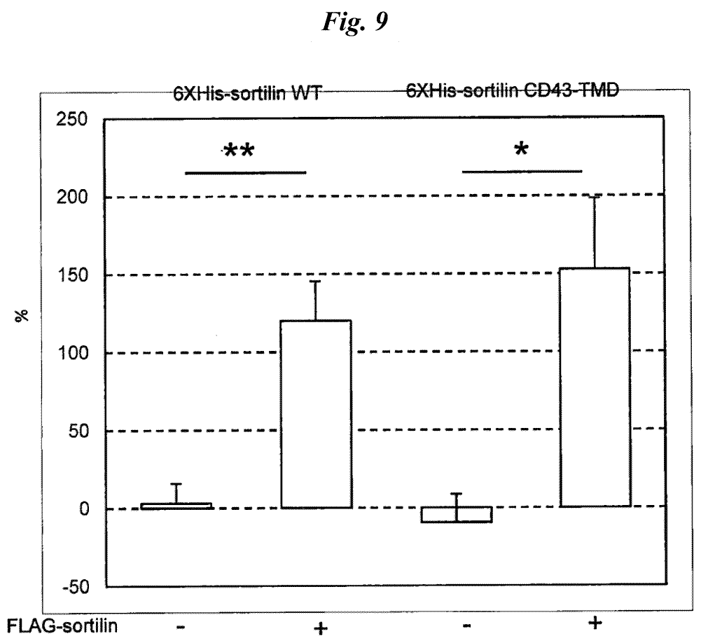

FIG. 8 shows a schematic of the molecular mechanisms of sortilin cleavage and trafficking to extracellular vesicles. Specifically, the schematic highlights that the dissociation of propeptide from sortilin that promotes dimerization. Dimerization with the intermolecular disulfide bond at Cys⁷⁸³ facilitates transport of dimerized sortilin to the extracellular vesicles. Furthermore, Cys⁷⁸³ is associated with dimerization and palmitoylation.

Palmitoylated sortilin is transported back to the Golgi apparatus. According to FIG. 8, the steps of sortilin trafficking within a cell comprise: (1) Propeptide is cleaved from sortilin; (2) propeptide binds to sortilin at different location and sortilin is transported through the Golgi apparatus; (3) sortilin forms homodimers with intermolecular disulfide bonds at 10CC domain and Cys⁷⁸³; (4) sortilin is incorporated to endosome by endocytosis; (5) palmitoylated sortilin monomer is transported back to Golgi by interaction with retromer; (6) sortilin homodimer is secreted by extracellular vesicles (microvesicles and/or exosomes); and (4) sortilin homodimer is shedded and secreted as soluble sortilin. Thus, the test agents provided herein can inhibit any portion of sortilin dimerization or trafficking as outlined by FIG. 8.

In one aspect, provided herein is a method for inhibiting intermolecular dimerization of sortilin in a subject comprising administering an agent to the subject in need thereof. In some embodiments, the inhibition of sortilin comprises administering a peptide to the subject in need thereof.

As used herein, the term “administer” refers to the placement of a composition into a subject by a method or route which results in at least partial localization of the pharmaceutically active agent at a desired site. The inhibitors can be administered in any suitable manner. The manner of administration can be chosen based on, for example, whether local or systemic treatment is desired, and on the area to be treated. Accordingly, a composition can be administered by any appropriate route which results in effective treatment in the subject, i.e., administration results in delivery to a desired location in the subject where at least a portion of the pharmaceutically active agent is delivered. Exemplary modes of administration include, but are not limited to, implant, injection, infusion, instillation, implantation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

In some embodiments, a composition provided herein can be implanted in a subject. As used herein, the term “implanted,” and grammatically related terms, refers to the positioning of the composition in a particular locus in the subject, either temporarily, semi-permanently, or permanently. The term does not require a permanent fixation of the composition in a particular position or location.

With respect to duration and frequency of administration or treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration once a month, once every two weeks, once a week, once every other day, daily, twice daily, three times daily or four or more times daily over a period of 1 week, 2 weeks, 3 weeks, 4 weeks, 1 month, 2 months, 3 months, 4 months, 5 months, or 6 months or more.

In some embodiments, the sortilin dimerization inhibitor can be co-administered to the subject in combination with a pharmaceutically active agent or therapeutic agent. Without limitations, the inhibitor can be administered before, concurrently, or after administration of the therapeutic agent. Thus, as used herein, the term “co-administer” refers to administration of two or more agents (e.g., the inhibitor and the pharmaceutically active agent) within a 24-hour period of each other, for example, as part of a clinical treatment regimen. In other embodiments, “co-administer” refers to administration within 12 hours, within 6 hours, within 5 hours, within 4 hours, within 3 hours, within 2 hours, within 1 hour, within 45, within 30 minutes, within 20, within 15 minutes, within 10 minutes, or within 5 minutes of each other. In other embodiments, “co-administer” refers to administration at the same time, either as part of a single formulation or as multiple formulations that are administered by the same or different routes. When the inhibitor and the pharmaceutically active agent are administered in different pharmaceutical compositions or at different times, routes of administration can be same or different.

Exemplary pharmaceutically active compound include, but are not limited to, those found in Harrison's Principles of Internal Medicine, 13th Edition, Eds. T. R. Harrison et al. McGraw-Hill N.Y., NY; Physicians' Desk Reference, 50th Edition, 1997, Oradell New Jersey, Medical Economics Co.; Pharmacological Basis of Therapeutics, 8th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

In some embodiments, pharmaceutically active agent can include those agents known in the art for treating cardiovascular calcification, such as vitamin D sterols and/or RENAGEL®. Vitamin D sterols can include calcitriol, alfacalcidol, doxercalciferol, maxacalcitol or paricalcitol.

In some embodiments, pharmaceutically active agent can include calcimimetics, vitamins and their analogs, antibiotics, lanthanum carbonate, lipid-lowering agents, such as a statin (e.g. LIPITOR®), other modulators of lipid profile (e.g., HDL-raising drugs), anti-hypertensives, anti-inflammatory agents (steroidal and non-steroidal), inhibitors of pro-inflammatory cytokine (ENBRELOR®, KINERET®), and cardiovascular agents.

In some embodiments, pharmaceutically active agent includes those agents known in the art for treatment of inflammation or inflammation-associated disorders.

In some embodiments, pharmaceutically active agent can by a bisphosphonate (Alendronate, Risendronate, Ibandronate, Zoledronic acid).

In some embodiments, pharmaceutically active agent can by a hormone-related agent.

In some embodiments, the pharmaceutically active agent is an anti-inflammatory agent. Exemplary anti-inflammatory agents include, but are not limited to, non-steroidal anti-inflammatory drugs (NSAIDs—such as aspirin, ibuprofen, or naproxen, coricosteroids (such as presnisone), anti-malarial medication (such as hydrochloroquine), methotrexrate, sulfasalazine, leflunomide, anti-TNF medications, cyclophosphamise, mycophenolate, and inhibitors of pro-inflammatory signaling pathways.

In some embodiments, the pharmaceutically active agent is an immune response modulator. As used herein, the term “immune response modulator” refers to compound (e.g., a small-molecule, antibody, peptide, nucleic acid, or gene therapy reagent) that inhibits autoimmune response in a subject. Without wishing to be bound by theory, an immune response modulator inhibits the autoimmune response by inhibiting the activity, activation, or expression of inflammatory cytokines (e.g., IL-12, IL-23 or IL-27), or STAT-4. Exemplary immune response modulators include, but are not limited to, members of the group consisting of Lisofylline (LSF) and the LSF analogs and derivatives described in U.S. Pat. No. 6,774,130, contents of which are herein incorporated by reference in their entirety.

In some embodiments, the pharmaceutically active agent is an antibiotic agent. The term “antibiotic” is used herein to describe a compound or composition which decreases the viability of a microorganism, or which inhibits the growth or reproduction of a microorganism. As used in this disclosure, an antibiotic is further intended to include an antimicrobial, bacteriostatic, or bactericidal agent. Exemplary antibiotics include, but are not limited to, penicillin, cephalosporins, penems, carbapenems, monobactams, aminoglycosides, sulfonamides, macrolides, tetracyclins, lincosides, quinolones, chloramphenicol, vancomycin, metronidazole, rifampin, isoniazid, spectinomycin, trimethoprim, sulfamethoxazole, and the like.

For administration to a subject, the inhibitor can be formulated in pharmaceutically acceptable compositions which comprise the inhibitor formulated together with one or more pharmaceutically acceptable carriers (additives) and/or diluents. The inhibitors can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; (9) nasally; or (10) local administration (e.g., drug eluting stent, pluronic gel). Additionally, the inhibitors can be implanted into a patient or injected using a drug delivery composition. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C2-C12 alcohols, such as ethanol; (26) lipid nanoparticles; and (27) other non-toxic compatible substances employed in pharmaceutical formulations. The carrier or excipient can include time delay material, such as glyceryl monostearate or glyceryl distearate alone or with a wax, or other materials well known in the art. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

The preparation of pharmaceutical compositions is well known in the art and has been described in many articles and textbooks, see e.g., Remington's Pharmaceutical Sciences, Gennaro A. R. ed., Mack Publishing Co., Easton, Pa., 1990, content of which of which is incorporated herein by reference in its entirety.

The pharmaceutical compositions can be made up in a solid form (including granules, powders or suppositories) or in a liquid form (e.g., solutions, suspensions, or emulsions). The pharmaceutical compositions can be subjected to conventional pharmaceutical operations such as sterilization and/or can contain conventional adjuvants, such as preservatives, stabilizers, wetting agents, emulsifiers, buffers etc.

Solid dosage forms for oral administration can include capsules, tablets, pills, powders, and granules. In such solid dosage forms, the active compound can be admixed with at least one inert diluent such as sucrose, lactose, or starch. Such dosage forms can also comprise, as in normal practice, additional substances other than inert diluents, e.g., lubricating agents such as magnesium stearate. In the case of capsules, tablets, and pills, the dosage forms can also comprise buffering agents. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting, sweetening, flavoring, and perfuming agents.

For purposes of parenteral administration, solutions in sesame or peanut oil or in aqueous propylene glycol can be employed, as well as sterile aqueous solutions of the corresponding water-soluble salts. Such aqueous solutions can be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art methods of preparing various pharmaceutical compositions with a certain amount of active ingredient are known, or will be apparent in light of this disclosure, to those skilled in this art.

The inhibitors can also be administered in controlled release formulations such as a slow release or a fast release formulation. Such controlled release formulations of the combination of this invention can be prepared using methods well known to those skilled in the art. The method of administration will be determined by the attendant physician or other person skilled in the art after an evaluation of the subject's conditions and requirements.

The amount of inhibitor that can be combined with a carrier material to produce a single dosage form will generally be that amount of the inhibitor that produces a therapeutic effect. Generally, out of one hundred percent, this amount will range from about 0.01% to 99% of inhibitor. In some embodiment, amount of the inhibitor in the composition can be selected from the range from about 0.1% to about 99% (w/w), from about 1% to about 90% (w/w), from about 2% to about 80% (w/w), from about 5% to about 75% (w/w), from about 5% to about 50% (w/w), from about 10% (w/w) to about 60% (w/w), from about 0.01% to about 95% (w/v), from about 0.1% to about 90% (w/w), from about 1% to about 85% (w/w), from about 10% to about 50% (w/w), from about 1% to about 99% (w/w), from about 0.05% to about 99% (w/w), from about 0.1% to about 90% (w/w), from about 0.5% to about 85% (w/w), or from about 5% to about 80% (w/w) of the total composition.

In some embodiments, the agent or composition administered to a subject comprises a therapeutically effective amount of the sortilin dimerization inhibitor for the treatment of an extracellular vesicle-associated disease (e.g. cardiovascular calcification).

As used herein, the term “therapeutically effective amount” means an amount of the therapeutic agent which is effective to provide a desired outcome. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in neurodegenerative disorders.

Furthermore, therapeutically effective amounts will vary, as recognized by those skilled in the art, depending on the specific disease treated, the route of administration, the excipient selected, and the possibility of combination therapy. In some embodiments, the therapeutically effective amount can be in a range between the ED50 and LD50 (a dose of a therapeutic agent at which about 50% of subjects taking it are killed). In some embodiments, the therapeutically effective amount can be in a range between the ED50 (a dose of a therapeutic agent at which a therapeutic effect is detected in at least about 50% of subjects taking it) and the TD50 (a dose at which toxicity occurs at about 50% of the cases). Guidance regarding the efficacy and dosage which will deliver a therapeutically effective amount of a compound can be obtained from animal models of condition to be treated.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized.

The therapeutically effective dose can be estimated initially from cell culture assays. A dose can be formulated in animal models to achieve a circulating plasma concentration range that includes the IC50 (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay. Examples of suitable bioassays include DNA replication assays, transcription based assays, and immunological assays.

The dosage can be determined by a physician and adjusted, as necessary, to suit observed effects of the treatment. Generally, the complex inhibitors are administered so that the inhibitor is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like. For protein based inhibitors (such as antibodies) one preferred dosage is 0.1 mg/kg to 100 mg/kg of body weight (generally 10 mg/kg to 20 mg/kg).

The dosage regimen for treating a disease condition, e.g., an EV-associated disease with the combination therapy disclosed herein can be selected in accordance with a variety of factors, including the type, age, weight, sex and medical condition of the patient, the severity of the disease, the route of administration, and the particular compound employed, and thus can vary widely.

In another aspect, provided herein is a method for identifying a test agent that modulates dimerization of sortilin. Generally, the method comprises: (i) contacting a cell with a test agent, wherein the cell expresses a first sortilin polypeptide comprising a first label, and a second sortilin polypeptide comprising a second label; (ii) detecting a distance or contact level between the first and second sortilin polypeptide expressed in the cell, wherein a change in distance or contact level relative to a control or reference level indicates the agent modulates dimerization of sortilin.

In some embodiments, an increase in the distance or contact level relative to a control or reference level indicates the compound inhibits dimerization of sortilin. In some other embodiments, a decrease in distance or contact level relative to a control or reference level indicates the compound increases dimerization of sortilin. In some embodiments, the control or reference level is the distance or contact level in a cell that has not been contacted with the test agent.

Any method available to one of skill in the art for determining distance or contact between two components can be used.

For example, the first and/or the second label can be a detectable label. As used herein, the term “detectable label” refers to a composition capable of producing a detectable signal indicative of the presence of a target. Detectable labels include any composition detectable by spectroscopic, photochemical, biochemical, immunochemical, electrical, optical or chemical means. Suitable labels include fluorescent molecules, radioisotopes, nucleotide chromophores, enzymes, substrates, chemiluminescent moieties, bioluminescent moieties, and the like. As such, a label is any composition detectable by spectroscopic, photochemical, biochemical, immunochemical, electrical, optical or chemical means needed for the methods and devices provided herein.

A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca2+Dye; Calcium Green-2 Ca2+; Calcium Green-5N Ca2+; Calcium Green-C18 Ca2+; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f, Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10 GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf, JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

Other exemplary detectable labels include luminescent and bioluminescent markers (e.g., biotin, luciferase (e.g., bacterial, firefly, click beetle and the like), luciferin, and aequorin), radiolabels (e.g., 3H, 125I, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

Means of detecting such labels are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label.

In some embodiments, the label is a heterologous protein. In some embodiments, the heterologous protein is a tag, such as a fluorescent protein. In some embodiments, the donor or acceptor is a soluble sortilin protein, a sortilin-derived propeptide, or sortilin-derived nucleic acid as provided herein that is conjugated to a fluorescent or protein tag. In some embodiments, the tag is conjugated to the C-terminus of the protein. In some embodiments, the tag is conjugated to the N-terminus of the protein. In some embodiments, the sortilin-derived propeptide is further conjugated to a magnetoresponsive bead for purification.

Such proteins can further facilitate tracking and/or visualization of the sortilin protein, dimers, and multimers. Additional non-limiting examples of heterologous protein tags that can be used in sortilin trafficking assays (e.g. FRET) include Histamine (HIS), sequence motif DYKDDDDK (where D=aspartic acid, Y=tyrosine, and K=lysine) or a FLAG tag, β-galactosidase, human influenza hemagglutinin (HA), OLLAS, c-myc, paramyxovirus of simian virus 5 epitope (V5) or any other protein epitope tag known in the art.

In some embodiments, sortilin dimerization can be detected or measured by analyzing the contacted the cell using Fluorescence Resonance Energy Transfer (FRET), e.g., Time-Resolved Fluorescence Resonance Energy Transfer (TR-FRET) or homogenous time resolved-fluorescence resonance energy transfer (HTRF). See, for example, Maurel et al. Anal. Biochem (2004), 253-262.

Fluorescence resonance energy transfer or Forster resonance energy transfer (FRET) is known in the art as a method to detect the proximity of two proteins on a cell surface (Maurel et al. Anal. Biochem (2004), 253-262) using a donor and acceptor fluorphore. The donor protein or nucleic acid can be labeled with a different fluorophore, protein tags, or epitope than the acceptor protein or nucleic acid. Thus, as the donor protein or nucleic acid contacts the acceptor second protein or nucleic acid, the change in fluorescence or a proteolytic cleavage can occur. This allows for detection of changes in the signal from the fluorophores used in this assay.

For example, one of the first or second label FRET donot and the other is a FRET acceptor, and the FRET signal is measured. Alternatively, a first ligand that is capable of binding with the first label and a second ligand that is capable of binding with the second label can be used. One of the first or second ligands can comprise a FRET dononr and the other can comprise a FRET acceptor. The contacted cell is further contacted with the ligands and the FRET signal is measured.

FRET signal is measured in the presence (e.g., in a cell contacted with the test agent) and absence (e.g., in a cell not contacted with the test agent) of the test agent. A change in FRET signal relative to a control or reference level indicating the agent modulates dimerization of sortilin. For example, an increase in FRET signal relative to a control or reference level indicates the compound inhibits dimerization of sortilin. Alternatively, a decrease in FRET signal relative to a control or reference level indicates the compound increases dimerization of sortilin. In some embodiments, the control or reference level is a FRET signal in a cell expressing both the first sortilin polypeptide and the second sortilin polypeptide. In some other embodiments, the control or reference level is a FRET signal in a cell expressing either the first sortilin polypeptide or the second sortilin polypeptide.

Any molecule capable of biding with the label can be used as the ligand. For example, the ligand can be an antibody, antigen binding fragment of an antibody, an aptamer, one part of a binding pair (e.g., biotin and avidin, or biotin and streptavidin). In some embodiments, the first and/or the second ligand is an antibody.

The FRET signal can be determined by a plate reader, confocal microscope, or any other detection method known in the art. For example, the FRET signal can be calculated as the ratio of counts per second (e.g. 665:620)×10,000, and % change of the FRET signal by tagged sortilin (e.g. 6×His-sortilin) expression. The signal can also be compared with an appropriate control tag. Sortlin can be considered to be dimerized when the FRET signal is 80% or more, 90% or more, 100% or more, 110% or more, 115% or more, 120% or more, 130% or more, 140% or more, 150% or more, 160% or more 170% or more, 180% or more, 190% or more, 200% or more and beyond. It is contemplated that these values can vary depending on the conditions of the assays such as the ratio of counts per second, the protein tag, or the control used.

It is contemplated that additional assays can be used to identify dimerization of sortilin which include but are not limited to enzyme-linked immunosorbent assay (ELISA), Western Blot, immunohistochemistry, protein-conjugated quantum dots, proteomics, mass spectrometry, Edman degradation, Matrix Assisted Laser Desorption/Ionization (MALDI), scanning electron microscopy, super resolution microscopy, transmission electron microscopy, or any other assay that identifies proteins, protein structure, composition, interactions, or localization known in the art.

As used herein, the term “test compound” refers to compounds and/or compositions that are to be screened for their ability to inhibit dimerization of sortilin. Test compounds may include a wide variety of different compounds, including chemical compounds, mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the test compound is a small molecule.

The number of possible test compounds runs into millions. Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule, Pharmacopia, graffinity, Panvera, Vitas-M Lab, Biomol International and Oxford. These libraries can be screened using the screening devices and methods provided herein. Chemical compound libraries such as those from NIH Roadmap, Molecular Libraries Screening Centers Network (MLSCN) can also be used.

Comprehensive list of compound libraries can be found on the web, for example, at www.broad.harvard.edu/chembio/platform/screening/compound_libraries/index.htm.

A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

Depending upon the particular embodiment being practiced, the test compounds may be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods provided herein, test compounds may be screened individually, or in groups. Group screening is particularly useful where hit rates for effective test compounds are expected to be low such that one would not expect more than one positive result for a given group.

Generally, compounds can be tested at any concentration that can inhibit and/or decrease dimerization of sortilin. In some embodiments, compounds are tested at a concentration in the range of from about 0.1 nM to about 1000 mM. Preferably the compound is tested in the range of from about 0.1 μM to about 10 μM.

In some embodiments, screening assay further comprises selecting the compound that inhibits or reduces sortilin dimerization. The test compound can inhibit or reduce sortilin dimerization by at least 10%, 20%, 30%, 40%, 50%, 50%, 70%, 80%, 90%, 95% or more relative to a control.

In some embodiments, the screening method is a high-throughput screening. High-throughput screening (HTS) is a method for scientific experimentation that uses robotics, data processing and control software, liquid handling devices, and sensitive detectors. High-Throughput Screening or HTS allows a researcher to quickly conduct millions of biochemical, genetic or pharmacological tests. High-Throughput Screening are well known to one skilled in the art, for example, those described in U.S. Pat. Nos. 5,976,813; 6,472,144; 6,692,856; 6,824,982; and 7,091,048, and contents of each of which is herein incorporated by reference in its entirety.

HTS uses automation to run a screen of an assay against a library of candidate compounds. An assay is a test for specific activity: usually inhibition or stimulation of a biochemical or biological mechanism. Typical HTS screening libraries or “decks” can contain from 100,000 to more than 2,000,000 compounds. The key labware or testing vessel of HTS is the microtiter plate: a small container, usually disposable and made of plastic, that features a grid of small, open divots called wells. Modern microplates for HTS generally have either 384, 1536, or 3456 wells. These are all multiples of 96, reflecting the original 96 well microplate with 8×12 9 mm spaced wells.

To prepare for an assay, the researcher fills each well of the plate with the appropriate reagents that he or she wishes to conduct the experiment with. After some incubation time has passed to allow the reagent to absorb, bind to, or otherwise react (or fail to react) with the compounds in the wells, measurements are taken across all the plate's wells, either manually or by a machine. Manual measurements are often necessary when the researcher is using microscopy to (for example) seek changes that a computer could not easily determine by itself. Otherwise, a specialized automated analysis machine can run a number of experiments on the wells such as colorimetric measurements, radioactivity counting, etc. In this case, the machine outputs the result of each experiment as a grid of numeric values, with each number mapping to the value obtained from a single well. A high-capacity analysis machine can measure dozens of plates in the space of a few minutes like this, generating thousands of experimental data points very quickly.

In another aspect, the invention provides a compound selected by the screening assay provided herein. It is to be understood that analogs, derivatives, and isomers of the compounds selected by the screening assays provided herein are also claimed herein.

In still another aspect, provided herein is a method for preparing dimeric soluble sortilin. Generally, the method comprises expressing a sortilin polypeptide comprising a first label in an extracellular domain from a cell and purifying the expressed sortilin polypeptide using affinity purification. The affinity purification can be based on the first label, for example, using a ligand capable of binding with the first label.

In some embodiments, the sortilin polypeptide comprises a complete or fragment of the amino acid sequence SEQ ID NO: 1 or SEQ ID NO: 2. In some embodiments, the sortilin protein is expressed by an expression vector.

Expression vectors can be constructed from, for example, pcDNA3.1 (+) vectors such as those commercially available from Thermo Fisher Scientific, Inc (Waltham, MA, USA). The term “vector”, as used herein, refers to a nucleic acid construct designed for delivery to a host cell or for transfer between different host cells. As used herein, a vector can be viral or non-viral. A vector can include, but is not limited to, a cloning vector, an expression vector, a plasmid, phage, transposon, cosmid, artificial chromosome, virus, virion, etc.

In some embodiments, the vector is integrative or non-integrative. In some embodiments, the non-integrative vector is an episomal vector, an EBNA1 vector, a minicircle vector, a non-integrative adenovirus, a non-integrative RNA, or a Sendai virus. In some embodiments, the vector is an episomal vector. In some embodiments, the vector is a lentiviral vector.

As used herein, the term “expression vector” refers to a vector that directs expression of an RNA or polypeptide (e.g., a sortilin inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as provided herein in place of non-essential viral genes. The vector and/or particle may be utilized for the purpose of transferring nucleic acids into cells either in vitro or in vivo. Numerous forms of viral vectors are known in the art.

Constructs of human sortilin (NM_002959.5) can be generated by inserting a protein tag as provided herein (e.g. FLAG (DYKDDDDK) or 6×His (HHHHHH) tag) into amino acids (e.g. 3 amino acids, S73A1330) behind the furin cleavage site (e.g. R74WRR77) using site-mutagenesis. Methods of cloning and expressing engineered proteins are known in the art. Other constructs of sortilin can be generated, such as sortilin CD43-TMD, by using an overlapping PCR strategy with a CD43 expression vector or an expression vector of sortilin with 3×FLAG at C-terminus.

An EV-deprived culture medium of cells with expressed sortilin (with the protein tag as provided herein) can be subjected to an affinity gel or solid support for purification, e.g., ANTI-FLAG® M2 from Sigma Aldrich (St. Louis, MO). Soluble sortilin with a protein tag can be eluted with a peptide. Purified soluble sortilin can then be dialyzed in phosphate buffer solution. Sortilin expression can be determine by Western Blot analysis or any other protein expression assay known in the art. Similarly, protein interactions can be determined by immunoprecipitation of sortilin with additional epitope-tagged proteins.

As provided herein, a “solid support” is any structure that can make contact with the target sortilin, dimeric sortilin, multimeric sortilin, sortilin-derived polypeptides, or epitope polypeptides conjugated to sortilin. The form of the solid support can comprise but are not limited to a scaffold, cartridge, column, filter, resin, or matrix, or bead. Non-limiting classes of materials that the solid support comprises include polymer, metal, ceramic, gels, paper, or glass.

In some embodiments, sortilin is bound directly or indirectly to the solid support. In some embodiments, the solid support comprises materials that include but are not limited to a polymer, metal, ceramic, gels, paper, or glass. The materials of the solid support can further comprise polystyrene, agarose, gelatin, alginate, iron oxide, stainless steel, gold nanobeads or particles, copper, silver chloride, polycarbonate, polydimethylsiloxane, polyethylene, acrylonitrile butadiene styrene, cyclo-olefin polymers, or cyclo-olefin copolymers, Sepharose™ resin.

In some embodiments, the solid support further comprises an element that is magnetoresponsive. In some embodiments, the magnetoresponsive element comprises magnetite, iron (III) oxide, samarium-cobalt, terfenol-D, or any other magnetic element described in the art.

The engineered sortilin protein can further be crosslinked to the membrane of a vesicle or cell to determine localization of the sortilin dimers. Chemical cross-linking can be carried out by incubating cells transiently overexpressing tagged-sortilin with bis (sulfosuccinimidyl) suberate (BS3), water-soluble, non-cleavable cross-linker at room temperature. The cells can be centrifuged at (e.g. at 1,000 rpm for 5 minutes) to remove the buffer including BS3, and washed with phosphate buffer solution. Then the cell can be lysed and prepared for Western blotting or immunoprecipitation as provided herein.

### Some Selected Definitions

Unless defined otherwise, all technical and scientific terms used herein have the same meaning as those commonly understood to one of ordinary skill in the art to which this invention pertains. Although any known methods, devices, and materials can be used in the practice or testing of the invention, the methods, devices, and materials in this regard are provided herein.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects provided herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as provided herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages can mean±1%.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with an EV-associated disease, or a biological sample that has not been contacted with an agent or composition disclosed herein).

As used herein, an “appropriate control” or “control” refers to an untreated, otherwise identical cell or population (e.g., a biological sample that was not contacted by an agent or composition provided herein, or not contacted in the same manner, e.g., for a different duration, as compared to a non-control cell).

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type provided herein and/or which will become apparent to those persons skilled in the art upon reading this disclosure and so forth.

Although methods and materials similar or equivalent to those provided herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

Some embodiments of the various aspect described herein can be described as in the following paragraphs:
1. A method for inhibiting or reducing trafficking of sortilin to an extracellular vesicle (EV) from a cell, the method comprising inhibiting covalent intermolecular dimerization of sortilin in the cell.
2. The method of paragraph 1, wherein said inhibiting comprises inhibiting formation of an intermolecular disulfide formation at Cys783 of SEQ ID NO: 1.
3. The method of paragraph 1 or 2, wherein said inhibiting comprises administering a peptide to the cell.
4. The method of paragraph 3, wherein said peptide is a sortilin-derived propeptide comprising an amino acid sequence of SEQ ID NO: 2.
5. The method of paragraph 3 or 4, wherein the peptide comprises at least one modification.
6. The method of any one of paragraphs 3-5, wherein the peptide is amidated, acetylated, cyclized, phosphorylated, glycosylated, nitrosylated, methylated, lipidated, or PEGylated.
7. The method of any one of paragraphs 3-6, wherein the peptide comprises at least one D amino acid, beta amino acid or modified peptide linkage.
8. The method of any one of paragraphs 3-7, wherein the peptide comprises at least one substituted amino acid.
9. The method of any one of paragraphs 1-8, wherein the cell is a leukocyte, lymphocyte, macrophage, natural killer cell, dendritic cell, T cell, or B cell.
10. The method of any one of paragraphs 1-9, wherein said inhibiting is in in vitro or ex vivo.
11. The method of any one of paragraphs 1-9, wherein said inhibiting is in vivo.
12. The method of paragraph 11, wherein said inhibiting is in a mammal.
13. The method of paragraph 11 or 12, wherein said inhibiting is in a subject having or suspected of having an extracellular vesicle associated disease (EV-associated disease).
14. The method of paragraph 13, wherein the EV-associated disease is selected from the group consisting of: calcific aortic valve disease, diabetes, systemic lupus erythematosus, ulcerative colitis, pulmonary fibrosis, nan-alcoholic fatty liver disease, osteoporosis, Alzheimer's disease, scleroderma, atherosclerosis, myocardial infarction, hypercholesterolemia, cancer, rheumatoid arthritis, and obesity.
15. The method of paragraph 14, where diabetes is type 1 diabetes, type 2 diabetes, or maturity onset diabetes in the young.
16. The method of paragraph 14, wherein cancer is lung cancer or pancreatic cancer.
17. A method of treating an extracellular vesicle associated disease in subject, comprising inhibiting inhibiting covalent intermolecular dimerization of sortilin in a cell in a subject in need thereof.
18. The method of paragraph 17, wherein said inhibiting comprises inhibiting formation of an intermolecular disulfide formation at Cys783 of SEQ ID NO: 1.
19. The method of paragraph 17 or 18, wherein said inhibiting comprises administering a peptide to the subject in need thereof.
20. The method of paragraph 19, wherein said peptide is a sortilin-derived propeptide comprising an amino acid sequence of SEQ ID NO: 2.
21. The method of paragraph 19 or 20, wherein the peptide comprises at least one modification.
22. The method of any one of paragraphs 19-21, wherein the peptide is amidated, acetylated, cyclized, phosphorylated, glycosylated, nitrosylated, methylated, lipidated, or PEGylated.
23. The method of any one of paragraphs 19-22, wherein the peptide comprises at least one D amino acid, beta amino acid or modified peptide linkage.
24. The method of any one of paragraphs 19-23, wherein the peptide comprises at least one substituted amino acid.
25. The method of any one of paragraphs 17-24, wherein the cell is a leukocyte, lymphocyte, macrophage, natural killer cell, dendritic cell, T cell, or B cell.
26. The method of any one of paragraphs 17-25, wherein said EV-associated disease is selected from the group consisting of calcific aortic valve disease, diabetes, systemic lupus erythematosus, ulcerative colitis, pulmonary fibrosis, nan-alcoholic fatty liver disease, osteoporosis, Alzheimer's disease, scleroderma, atherosclerosis, myocardial infarction, hypercholesterolemia, cancer, rheumatoid arthritis, and obesity.
27. The method of paragraph 26, where diabetes is type 1 diabetes, type 2 diabetes, or maturity onset diabetes in the young.
28. The method of paragraph 26, wherein cancer is lung cancer or pancreatic cancer.
29. A method for identifying a test agent that modulates dimerization of sortilin, the method comprising:
   (i) contacting a cell with a test agent, wherein the cell expresses a first sortilin polypeptide comprising a first label, and a second sortilin polypeptide comprising a second label;
   (ii) detecting a contact level between the first and second sortilin polypeptide expressed in the cell,
   wherein a change in contact level relative to a control or reference level indicates the agent modulates dimerization of sortilin.
30. The method of paragraph 29, wherein said detecting comprises analyzing the cell contacted in step (i) using Time-Resolved Fluorescence Resonance Energy Transfer (TR-FRET), and wherein a change in FRET signal relative to a control or reference level indicates the agent modulates dimerization of sortilin.
31. The method of paragraph 29 or 30, wherein an increase in FRET signal relative to a control or reference level indicates the compound inhibits dimerization of sortilin.
32. The method of paragraph 29 or 30, wherein a decrease in FRET signal relative to a control or reference level indicates the compound increases dimerization of sortilin.
33. The method of any one of paragraphs 30-32, wherein the control or reference level is a FRET signal in a cell expressing either the first sortilin polypeptide or the second sortilin polypeptide.
34. The method of any one of paragraphs 29-33, wherein said detecting comprises contacting the cell with a first ligand and a second ligand, wherein the first ligand is capable of binding with the first label and is conjugated with a with a Fluorescence Resonance Energy Transfer (FRET) donor, and wherein the second ligand is capable of binding with the second label and is conjugated with a FRET acceptor.
35. The method of paragraph 34, wherein the first or second ligand is an antibody.
36. The method of any one of paragraphs 29-35, wherein the agent inhibits dimerization of sortilin.
37. The method of any one of paragraphs 29-35, wherein the agent increases dimerization of sortilin.
38. A method for preparing dimeric soluble sortilin, the method comprising:
   (i) expressing a sortilin polypeptide comprising a first label in an extracellular domain from a cell;
   (ii) purifying the expressed sortilin polypeptide via affinity purification using a ligand capable of binding with the first label.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1: Dimerization of Sortilin Regulates its Trafficking to Extracellular Vesicles

Extracellular vesicles (EVs) play a critical role in intercellular communication by transferring microRNAs, lipids and proteins to neighboring cells. Sortilin, a sorting receptor that directs target proteins to the secretary or endocytic compartments of cells, is found in both EVs and cells. In many human diseases, including cancer and cardiovascular diseases, sortilin expression levels are atypically increased. To elucidate the relationship between cardiovascular disease, particularly vascular calcification, and sortilin expression levels, the trafficking of sortilin in both intracellular and extracellular milieu was explored. It has been demonstrated previously that sortilin promotes vascular calcification via its trafficking of tissue-nonspecific alkaline phosphatase (TNAP) to EVs. Although recent reports have noted that sortilin is regulated by multiple post-translational modifications, the precise mechanisms of sortilin trafficking still need to be elucidated. As provided herein, sortilin forms homodimers with an intermolecular disulfide bond at Cysteine 783 (Cys⁷⁸³) residue, indicating that Cys⁷⁸³ can be shared via palmitoylation and an intermolecular disulfide bond, as Cys⁷⁸³ can be palmitoylated. See SEQ ID NO: 1 for the amino acid sequence of sortilin.

Formation of the intermolecular disulfide bond can lead to trafficking of sortilin to EVs by preventing palmitoylation, and promotes trafficking of sortilin to the Golgi apparatus. Moreover, it was observed that sortilin-derived propeptide decreased sortilin homodimers within EVs. In conclusion, sortilin is transported to EVs via the formation of homodimers with an intermolecular disulfide bond, which is endogenously regulated by its own propeptide. Therefore, inhibiting dimerization of sortilin can act as a new therapeutic strategy for the treatment of EV-associated diseases, including vascular calcification and cancer, among others.

Intercellular communication, an essential hallmark of multicellular organisms, can be mediated through direct cell-cell contact or the transfer of secreted molecules (1). In the last two decades, a new mechanism for intercellular communication has emerged that involves intercellular transfer of extracellular vesicles (EVs), such as exosomes, which have the ability to transfer their cellular content to neighboring cells and to modify the cellular microenvironment (2) (3). The role of EVs is likely to be dictated by the vesicle cargo, which can be composed of microRNAs, RNAs, lipids and/or proteins. However, the function of some of these proteins in EVs and how they affect various diseases need to be further elucidated.

Sortilin, which is ubiquitously expressed and essential for proper function of many tissue and cell types, is a sorting receptor that directs target proteins, including growth factors, signaling receptors, and enzymes, to their destined location in the secretory or endocytic compartments of cells (4). Sortilin has conversely also emerged as a major cause of malignancies in a range of diseases, including cancer (5) (6) (7) (8), Type 2 diabetes mellitus (9), hypercholesterolemia (10) (11) (12), atherosclerosis (13) (14), and neurodegenerative disorders (15) (16) like Alzheimer's disease (17) (18). The atypical increase in intracellular trafficking by sortilin, and its subsequent lysosomal degradation (15) or secretion (10) (14), has been linked to the pathogenesis of the above diseases. In addition, recent studies have shown that sortilin can convey causative molecules of diseases to the extracellular space via EVs: 1) Previous studies showed that sortilin transports tyrosine kinases to neighboring cells through exosome transfer, promoting tumorigenesis via activation of angiogenesis (6), and 2) previous research has demonstrated that sortilin promotes vascular calcification via its trafficking and loading of tissue-nonspecific alkaline phosphatase (TNAP) into EVs (19).

Therefore, the major objective is to understand the process that facilitates the transport of sortilin into EVs. Addressing this question can help to discover new therapeutic approaches for EV-associated diseases. Although multiple post-translational modifications, including phosphorylation (19), and ubiquitination (20) (21), palmitoylation (22), can regulate functions of sortilin, the mechanisms regulating sortilin trafficking have yet to be fully understood. Since the trafficking of receptors, such as G-protein coupled-receptors (23) and type I transmembrane proteins (24) (25), can be regulated by dimerization, it is contemplated that dimerization can be a major regulator of sortilin trafficking to EVs. Provided herein is the first evidence that sortilin forms homodimers, thereby facilitating its trafficking to EVs. Specifically, the results showed that: 1) sortilin forms homodimers with an intermolecular disulfide bond at Cys⁷⁸³; 2) mutation of Cys⁷⁸³ abolishes transport of dimerized sortilin to EVs; and 3) inhibition of palmitoylation at Cys⁷⁸³ increases sortilin homodimers. The results indicate that Cys⁷⁸³ can be involved in both palmitoylation and the formation of the intermolecular disulfide bond, which can regulate the trafficking of sortilin to EVs, thus preventing palmitoylation, which promotes trafficking of sortilin to the Golgi apparatus (22). Moreover, sortilin-derived propeptide decreases sortilin homodimers in EVs. Therefore, based on these findings, the mechanism regulating trafficking of sortilin via its dimerization with an intermolecular disulfide bond is regulated via ligand binding in the extracellular domain.

### Sortilin Forms Homodimers on the Cell Surface

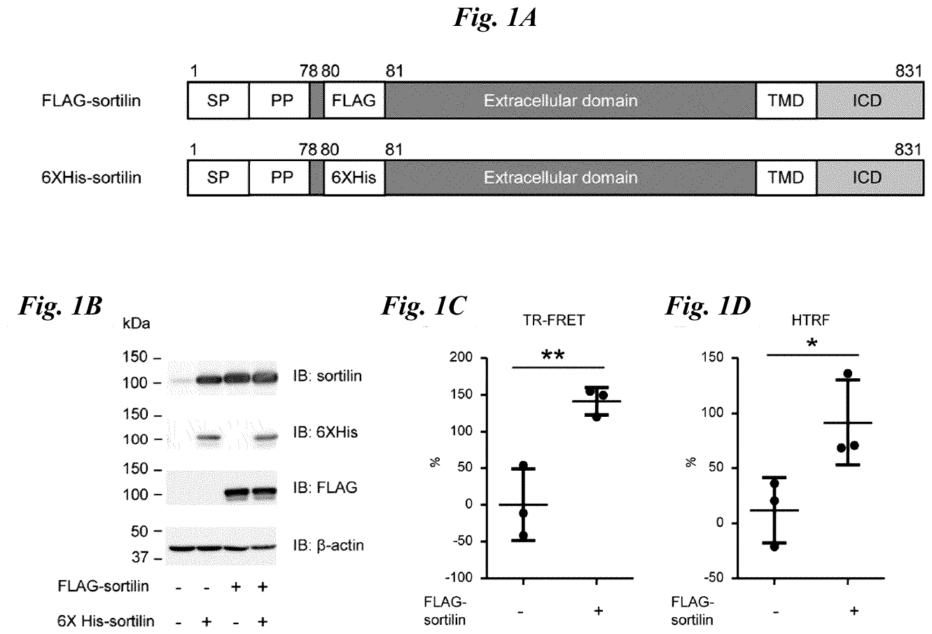

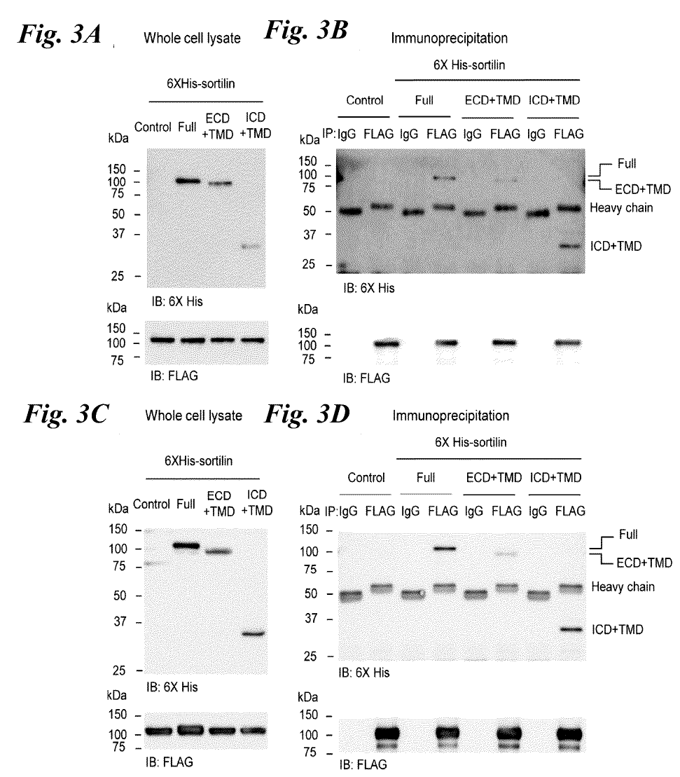

Time-resolved fluorescence energy transfer (TR-FRET) assay was performed to detect sortilin homodimerization. Expression vectors of FLAG-sortilin and 6×His-sortilin were constructed for the TR-FRET assay (FIG. 1A). FLAG-tag and 6×His-tag were placed following propeptide and 3 amino acids (Ser-Ala-Pro) to detect the extracellular domains of FLAG-sortilin and 6×His-sortilin after propeptide cleavage (FIG. 1A). Both FLAG-sortilin and 6×His-sortilin were overexpressed in HEK293 cells. Protein expression of FLAG-sortilin and 6×His-sortilin was validated in the western blotting (FIG. 1B). This co-expression increased the FRET signal when compared to HEK293 cells overexpressing only 6×His-sortilin (FIG. 1C), indicating that sortilin forms homodimers on the cell surface. Also, an increased FRET signal was detected in homogenous TR-FRET (HTRF), a result which aligns with previous reports (26) (FIG. 3D). These results indicate that the FRET assay is effective in screening for molecules involved in sortilin dimerization.

Sortilin Forms Homodimers in the Extracellular and Intracellular Domains with Intermolecular Disulfide Bonds

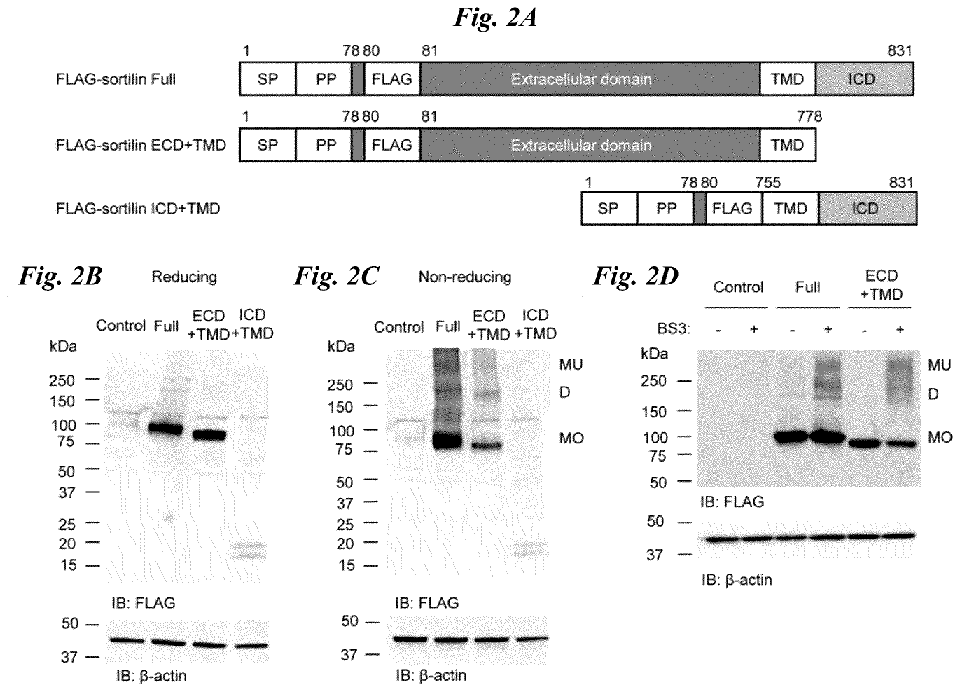

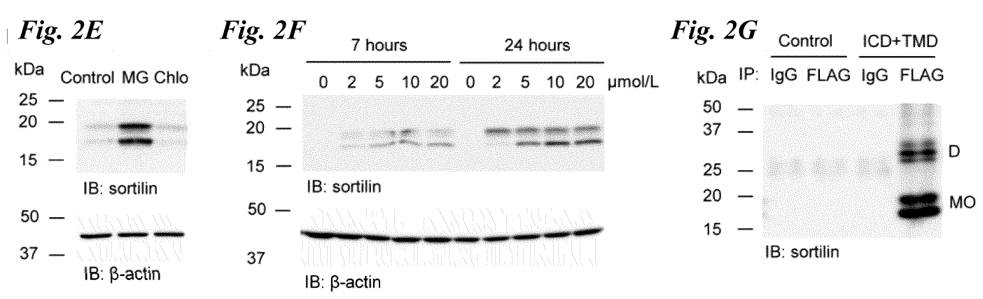

To investigate whether the extracellular domain (ECD) or intracellular domain (ICD) is responsible for the dimerization of sortilin, expression vectors of FLAG-sortilin ECD plus transmembrane domain (TMD) and ICD+TMD were constructed and then overexpressed in HEK293 cells (FIG. 2A). In reducing western blotting, protein expressions of FLAG-sortilin Full, ECD+TMD and ICD+TMD were detected as bands of plausible molecular size (FIG. 2B). In non-reducing western blotting where disulfide bonds can be retained, FLAG-sortilin Full and ECD+TMD expressed a couple of bands (FIG. 2C). A band of 75-100 kDa was detected as monomers (FIG. 2C). Bands of approximately 200 kDa and higher molecular weight were detected as homodimers and multimers with intermolecular disulfide bonds (FIG. 2C). Next, FLAG-sortilin Full and ECD+TMD were cross-linked using water-soluble, non-cleavable cross-linker, BS3, in HEK293 cells. Both bands of homodimers and multimers appeared via the cross-linking (FIG. 2D). These data suggest that sortilin forms homodimers and multimers in the extracellular domain. Dimerization of FLAG-sortilin ICD+TMD was not clearly detected in the non-reducing western blotting using whole cell lysate (FIG. 2C) potentially due to low protein expression levels of FLAG-sortilin ICD+TMD (FIGS. 2B, 2C). Also, protein expression of FLAG-sortilin ICD+TMD was lower in HEK293 stably expressing FLAG-sortilin ICD+TMD (FLAG-sortilin ICD+TMD BEK293 cells). Therefore, it was determined whether FLAG-sortilin ICD+TMD can undergo degradation in proteasome and lysosome by adding proteasome inhibitor, MG-132, and lysosome inhibitor, chloroquine, to FLAG-sortilin ICD+TMD BEK293 cells, respectively. MG-132 increased protein expression of FLAG-sortilin ICD+TMD in a time- and concentration-dependent manner, while chloroquine did not, suggesting that FLAG-sortilin ICD+TMD is degraded in proteasome (FIGS. 2E, 2F). To detect homodimers of RAG-sortilin ICD+TMD, incubation with MG-132 was performed and immunoprecipitation with anti-FLAG antibody. In non-reducing western blotting, bands with molecular size approximately twice as high as monomers of FLAG-sortilin ICD+TMD were detected (FIG. 2G). These data indicate that sortilin forms homodimers with an intermolecular disulfide bond in the intracellular domain.

### The Transmembrane Domain of Sortilin Forms Homodimers Via Noncovalent Interaction

To confirm dimerization of sortilin in ECD and ICD, immunoprecipitation was performed using HEK293 cells stably overexpressing FLAG-sortilin Full (FLAG-sortilin Full BEK293 cells or FLAG-sortilin HEK293 cells) where 6×His-sortilin Full, ECD+TMD or ICD+TMD were transiently overexpressed, respectively (FIGS. 3A, 3B). 6×His-sortilin Full, ECD+TMD and ICD+TMD were precipitated with FLAG-sortilin Full (FIG. 3B). Since their constructs have a transmembrane domain, the possibility that the transmembrane domain forms dimers remained. Therefore, to investigate dimerization of a transmembrane domain, immunoprecipitation was carried out using HEK293 cells stably overexpressing FLAG-sortilin ECD+TMD (FLAG-sortilin ECD+TMD BEK293 cells) where 6×His-sortilin ICD+TMD was transiently overexpressed (FIGS. 3C, 3D). 6×His-sortilin Full and ECD+TMD were also overexpressed as a positive control (FIGS. 3C, 3D). 6×His-sortilin Full, ECD+TMD and ICD+TMD were precipitated with FLAG-sortilin ECD+TMD (FIG. 3D). Binding of FLAG-sortilin ECD+TMD and 6×His-sortilin ICD+TMD indicate that sortilin can form homodimers via noncovalent interaction in the transmembrane domain.

Substituting the Transmembrane Domain of Sortilin with the Corresponding Domain of CD43 does not Generate a Monomeric Form of Sortilin

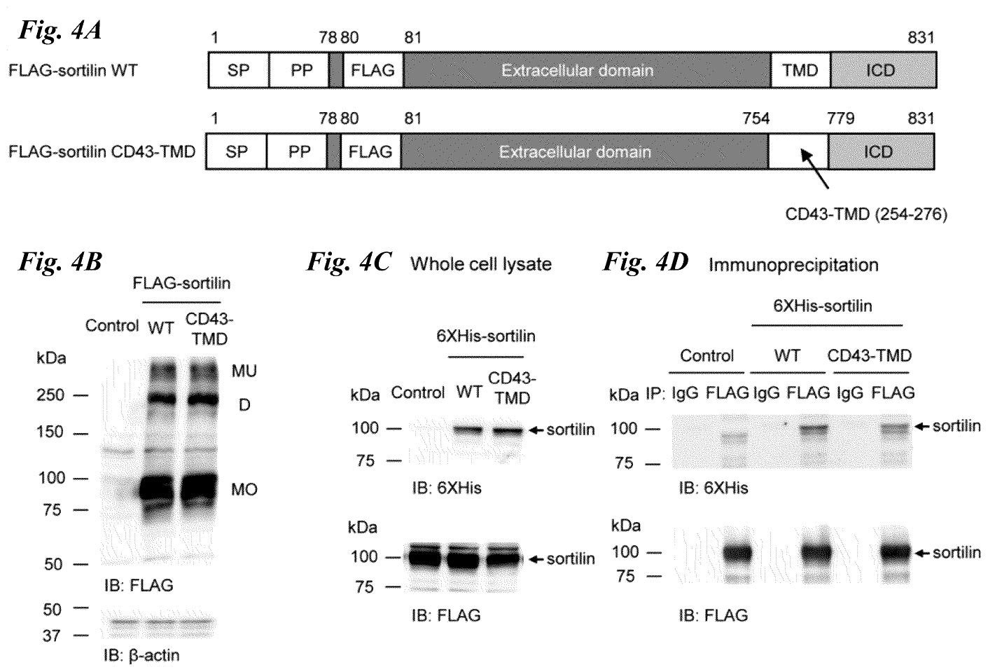

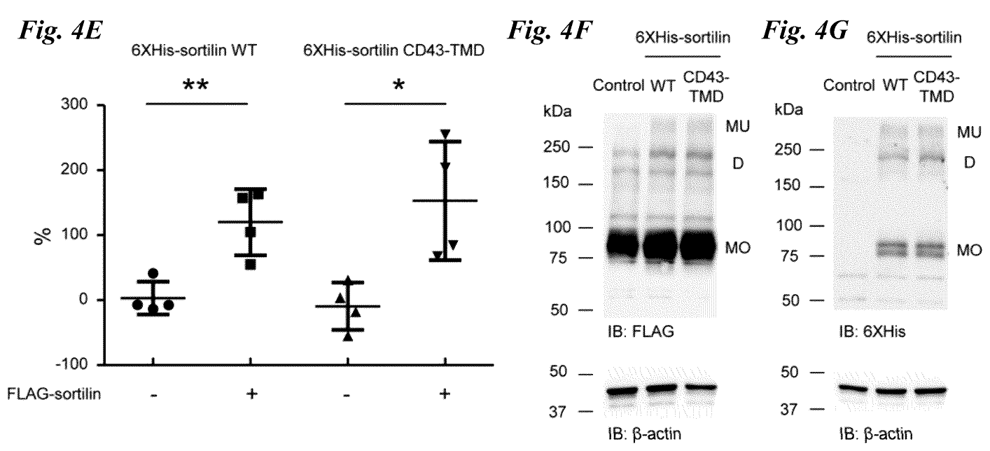

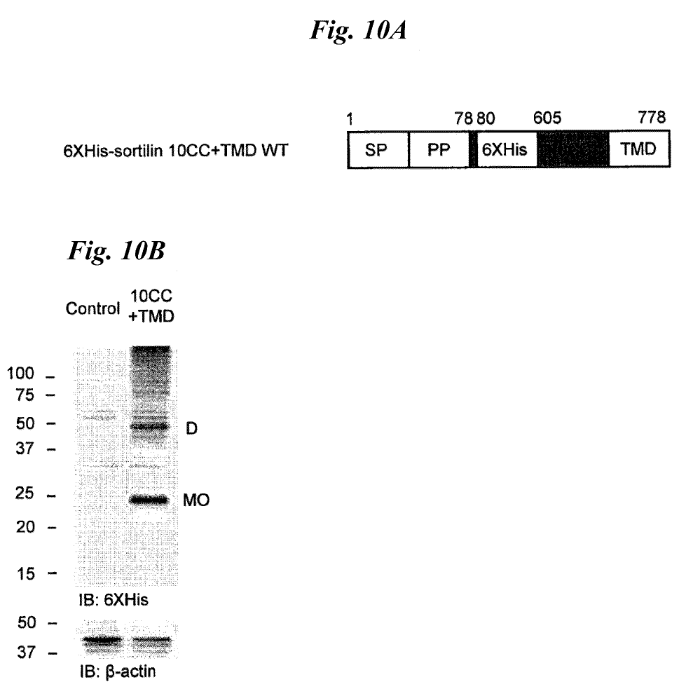

To investigate the contribution of the transmembrane domain of sortilin on dimerization, the transmembrane domain was replaced with that of CD43, which does not form homodimers (sortilin CD43-TMD), as was previously reported (FIG. 4A) (25). Sortilin CD43-TMD formed homodimers in the non-reducing western blotting (FIG. 4B). Also, 6×His-sortilin CD43-TMD was precipitated with FLAG-sortilin wild-type in the immunoprecipitation experiment of HEK293 cells as well as 6×His-sortilin wild-type (FIGS. 4C, 4D), and co-expression of FLAG-sortilin and 6×His-sortilin CD43-TMD increased FRET signal in HEK293 cells as well as 6×His-sortilin wild-type (FIG. 10). These data suggest that inhibiting dimerization of the transmembrane domain is not sufficient to suppress dimerization of sortilin possibly due to dimerization of the intracellular and extracellular domain.

### Mutation of Cys783 Abolished Dimerization of Sortilin

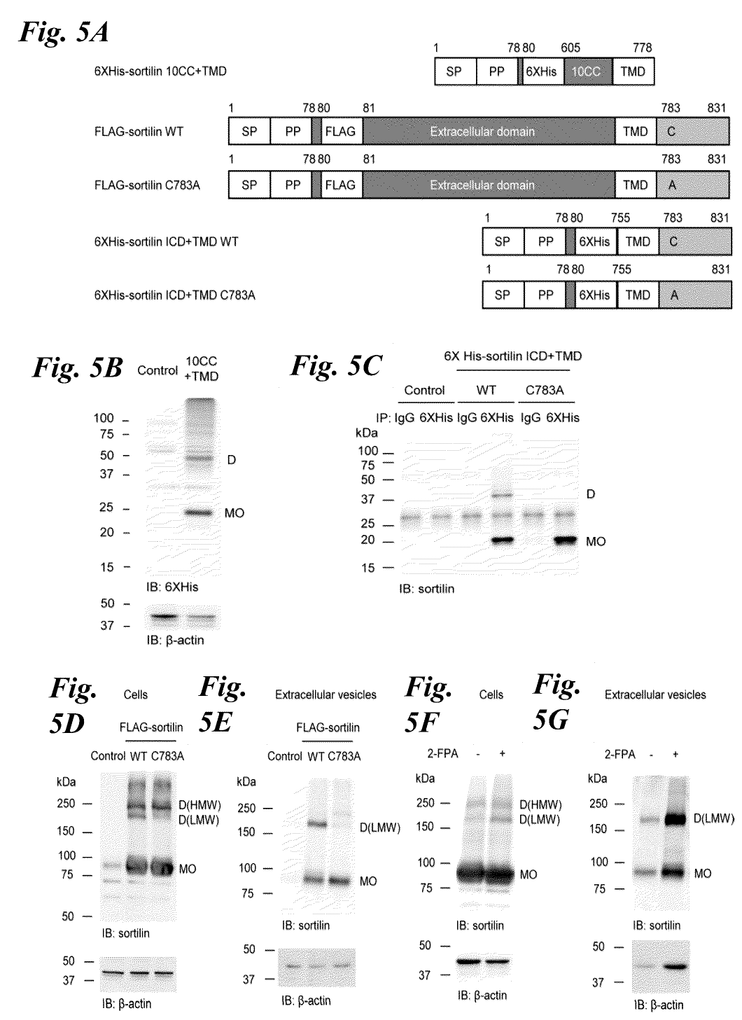

Previous reports showed that cysteines play an important role in maintaining the structure of sortilin since they form intramolecular disulfide bonds in the extracellular domain of sortilin (27). In addition, in this study, it is demonstrated that intermolecular disulfide bonds are formed within homodimers. Although the cysteines responsible for the formation of intermolecular disulfide bonds in the extracellular domain have been not yet identified, the 10CC domain formed dimers in the non-reducing western blotting (FIG. 10), indicating that intermolecular disulfide bonds can be formed within the 10CC domain. Next, the intermolecular disulfide bond was examined in the intracellular domain. Since sortilin has only one cysteine (Cys⁷⁸³) in the intracellular domain, the cysteine was expected to form an intermolecular disulfide bond for dimerization in the intracellular domain. Cys⁷⁸³ was replaced by alanine (C783A) in 6×His-sortilin ICD+TMD and FLAG-sortilin Full (FIG. 5A). 6×His-sortilin ICD+TMD C783A did not form homodimers in HEK293 cells (FIG. 5B). Surprisingly, FLAG-sortilin C783A decreased only homodimers of low molecular weight in HEK293 cells, although it did not change those of high molecular weight and multimers (FIG. 5C). The homodimers of low molecular weight were mainly transported to EVs, while homodimers of high molecular weight and multimers were mainly not transported to EVs (FIG. 5D). Therefore, FLAG-sortilin C783A significantly decreased transport of dimerized sortilin to EVs (FIG. 5D). Since Cys⁷⁸³ was reported to be palmitoylated (22), the connection between palmitoylation and intermolecular disulfide bond was studied by incubating FLAG-sortilin HEK293 cells with an inhibitor of palmitoylation, 2-fluoropalmitic acid (2-FPA) (22), which increased only homodimers of lower molecular size in the cells (FIG. 5E). These data indicate that Cys⁷⁸³ can be shared by palmitoylation and the intermolecular disulfide bond. Also, 2-FPA increased dimerization of FLAG-sortilin in the EVs (FIG. 5E), suggesting that a decrease of the homodimers in the EVs via the mutation of Cys⁷⁸³ is due to a decrease in dimerization, not palmitoylation.

### Binding of Sortilin-Derived Propeptide Suppresses Dimerization of Sortilin.

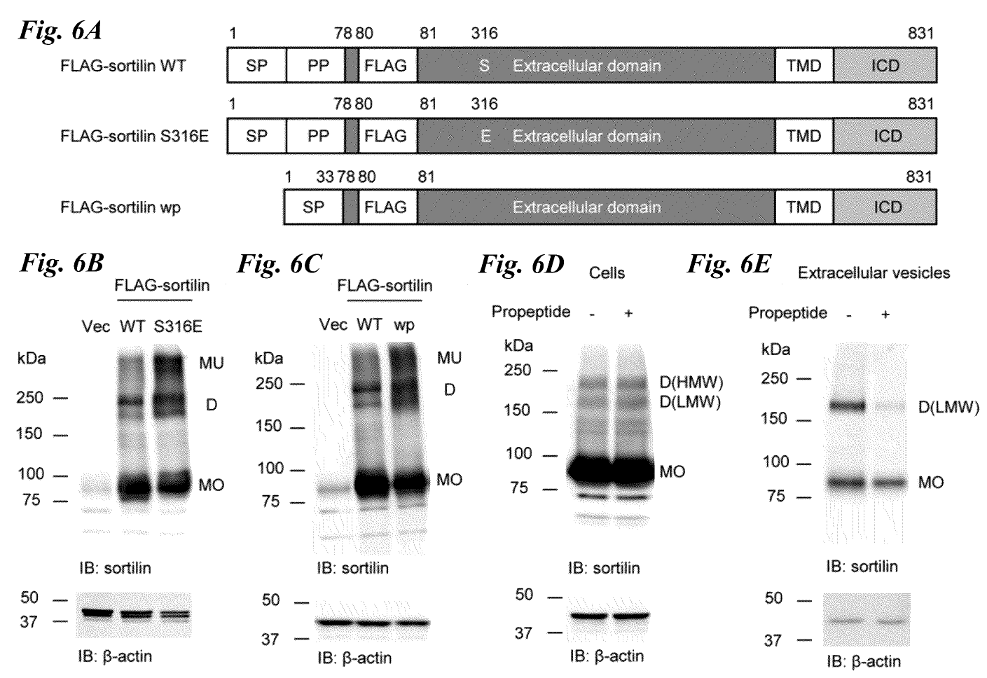

The structure of the Vps 10p domain in both sortilin and SorLA has been reported (28) (29). SorLA has different configuration in a ligand-free state or propeptide-bound state. Similar changes can take place in sortilin, and these different states can form monomers and homodimers of sortilin. Since the S316E mutation inhibits binding with sortilin-derived propeptide (29), a S316E mutant was used (FIG. 6A) to investigate the effects of the propeptide binding on dimerization. S316E increased dimerization in HEK293 cells concomitantly with a decrease in monomers (FIG. 6B). To further validate the effect of the propeptide binding on dimerization, sortilin without propeptide (wp) (27) was constructed (FIG. 6A) and overexpressed in HEK293 cells. Sortilin wp also increased the dimerization in HEK293 cells, concomitantly with a decrease in monomers (FIG. 6C). Also, the addition of sortilin-derived propeptide decreased dimerization in the extracellular vesicles of FLAG-sortilin HEK293 cells (FIG. 6E), though it did not affect dimerization in the cells (FIG. 6D).

### Soluble Sortilin Exists as Homodimers

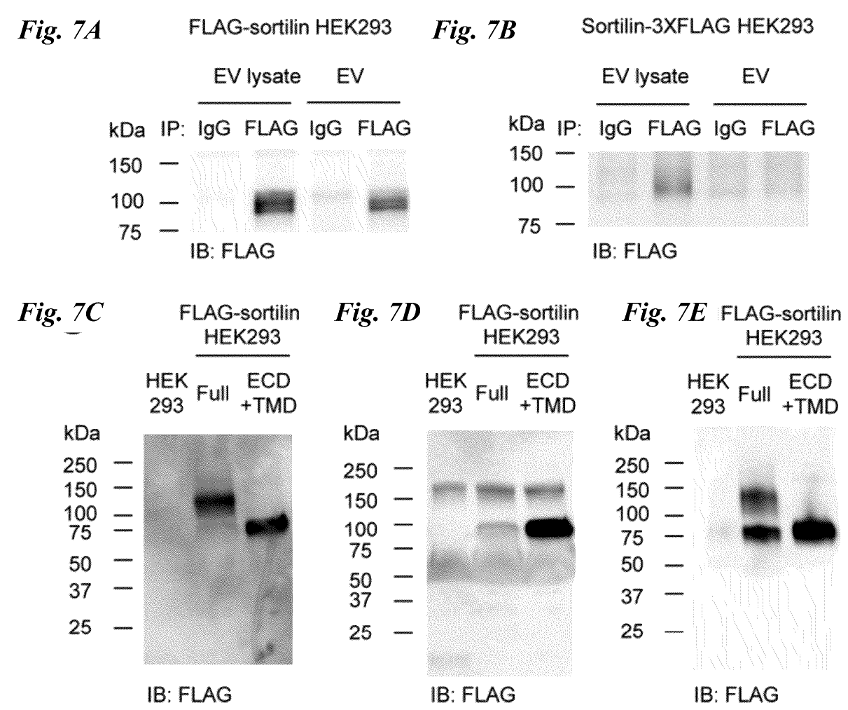

It has been reported that serum sortilin levels associate with cardiovascular risk, such as aortic calcification (30) and atherothrombosis (31), as well as depression (32). It has been also demonstrated that soluble sortilin can activate the survival of cancer cells (5) (33). Therefore, it is important to understand whether soluble sortilin can contribute to diseases via the formation of monomers and/or homodimers. In addition, it is critical to determine the orientation of sortilin on the EV membrane for the detection of soluble sortilin and sortilin in EVs. Since serum sortilin levels has been measured using antibodies against the extracellular domain of sortilin, these antibodies can detect both as long as the extracellular domain of sortilin locates outside of EVs. Therefore, to determine the orientation of sortilin on the EV membrane, an immunoprecipitation assay was performed using EVs secreted from FLAG-sortilin Full HEK293 cells and HEK293 cells stably expressing sortilin with 3×FLAG at C-terminus (sortilin-3×FLAG). FLAG-sortilin was detected in EVs and the lysate (FIG. 7A), but sortilin-3×FLAG was detected in the lysate only (FIG. 7B), suggesting that the extracellular domain of sortilin located outside of EVs. Next, to detect forms of soluble sortilin secreted from FLAG-sortilin HEK293 cells, a non-reducing western blotting was performed using EV-deprived culture medium. The molecular size of soluble sortilin was calculated as approximately 120 kDa in the non-reducing western blotting (FIG. 7C). Since this size is higher than that detected in the reducing western blotting (FIG. 7D), the band of 120 kDa was detected as homodimers. Also, forms of soluble sortilin secreted from FLAG-sortilin ECD+TMD HEK293 cells were investigated since the intracellular domain of sortilin can be cleaved (6) (34). Soluble sortilin from FLAG-sortilin ECD+TMD HEK293 cells was detected as a band at approximately 80 kDa (FIG. 7C) in the form of monomers. Next, soluble sortilin was purified using an anti-FLAG antibody affinity column using EV-deprived culture medium. Soluble sortilin secreted from FLAG-sortilin HEK293 cells was detected as bands of 80 and 120 kDa, which associate with dimers of soluble sortilin that partially changed their form to monomers during the process of purification (FIG. 7E). These data indicate that the band of 120 kDa represents the soluble sortilin dimer.

### DISCUSSION

As provided herein, sortilin promotes vascular calcification via its trafficking of tissue-nonspecific alkaline phosphatase (TNAP), a facilitator of calcification, to EVs (19). Also, other groups have reported that sortilin promotes exosome release and forms a complex with two tyrosine receptors, tropomyosin-related kinase B (TrkB) and epidermal growth factor receptor (EGFR), which can play an important role in the control of the cancer cell microenvironment and tumor angiogenesis (6). Given these results, the major objective of the study was to understand how sortilin is transported to EVs in order to potentially inhibit the atypical expression levels observed in multiple diseases, including cardiovascular disease.

Accordingly, sortilin forms homodimers with an intermolecular disulfide bond. In the intracellular domain of sortilin, Cys⁷⁸³ forms an intermolecular disulfide bond to generate homodimers. Since Cys⁷⁸³ has been reported to be palmitoylated (22), formation of an intermolecular disulfide bond can compete with palmitoylation at Cys⁷⁸³. The palmitoylation inhibitor increased sortilin dimerization. Furthermore, dimerized sortilin with an intermolecular disulfide bond at Cys⁷⁸³ acts as the main dimer transported to EVs, and loss of the intermolecular disulfide bond at Cys⁷⁸³ via mutation can cease the transport of dimerized sortilin to EVs. Since the palmitoylation inhibitor increased transport of dimerized sortilin to EVs, formation of an intermolecular disulfide bond at Cys⁷⁸³ residue can facilitate transport of dimerized sortilin to EVs, possibly due to the fact that palmitoylation accelerates trafficking to the Golgi apparatus (22) (FIG. 8).

The immunoprecipitation experiments showed that, in the transmembrane domain of sortilin, noncovalent interaction can occur to form homodimers. However, inhibiting binding in the transmembrane domain was not sufficient to suppress dimerization of sortilin. This can be explained by sortilin covalent binding in the intracellular and extracellular domain, while, concurrently, other type I transmembrane proteins such as PSGL-1 (25) and amyloid precursor protein (35) form homodimers through the transmembrane domain.

In the extracellular domain, 10CC domain showed intermolecular disulfide bonds. The 10CC domain has 10 cysteines, which form intramolecular disulfide bonds (27). However, the results support the possibility that some of cysteines in the 10CC domain contribute to the formation of intermolecular disulfide bonds for homodimers. Since C783A mutant decreased only homodimers of low molecular weight, homodimers with cysteines in the 10CC domain were formed, and the C783A mutant existed as homodimers of high molecular weight and multimers. To not be bound by a particular theory, it was contemplated previously that the interaction of propeptide binding site and 10CC domain occurs (29). It was further contemplated that binding of sortilin-derived propeptide affects dimerization. Since the structure of sorLA, which, similar to sortilin, has a VpslOp domain, can change in a ligand-free state or propeptide-bound state (28), and that these two different states can contribute to the formation of either monomers or homodimers of sortilin (FIG. 8). Also shown was that both sortilin 5316E and sortilin wp increased formation of homodimers, while the addition of propeptide reduced them. Since the ligands regulated dimerization of sortilin, future studies should investigate the effects of other ligands, such as progranulin (17) and neurotensin (36), in addition to sortilin-derived propeptide, on sortilin dimerization.

Here, it was demonstrated that soluble sortilin can exist as homodimers. Moreover, the intracellular domain is essential for the dimerization of soluble sortilin. Previous studies used sortilin overexpressed as recombinant protein lacking in transmembrane and intracellular domains, indicating that soluble sortilin has been present in form of monomers (33) (37). As demonstrated herein, is a method to produce dimerized soluble sortilin. Since, in the purification process of dimerized soluble sortilin, some of intermolecular disulfide bonds can be destroyed, resulting in monomer formation, the purification procedure can be improved. In addition, dimerized soluble sortilin can be used to examine physiological and pathological functions of its soluble form.

The findings of dimerized soluble sortilin have important implications in a clinical setting, since serum sortilin can act as a biomarker to detect cardiovascular and neurologic diseases (30) (31) (32). Therefore, it is important to clarify the differences between monomers and dimers of soluble sortilin, and the monomer/dimer ratio in serum for these diseases. This can allow for a more accurate diagnosis for the diseases, similar to the detection of high molecular weight adiponectin for metabolic syndrome (38) (39). Also, it was determined that the extracellular domain of sortilin located outside of EVs. This finding validates the possibility that the detection of sortilin-positive EVs can be possible using antibodies against the extracellular domain. Clarifying the association of sortilin-positive EVs with various diseases can be useful as a clinically-relevant surrogate for disease progression. In fact, the potential for using exosomal proteins in disease diagnosis and prognosis prediction has been increasing (40).

In conclusion, it was demonstrated that sortilin forms homodimers, which can play an important role in the trafficking of sortilin to the EVs and can be regulated using an intermolecular disulfide bond. In addition, sortilin-derived propeptide can control the dimerization of sortilin, and therefore the possibility of its regulation via ligand binding in the extracellular domain. Based on these findings, it is expected that molecules inhibiting sortilin dimerization, such as small molecule compounds, antibodies and peptides, can provide new therapeutic approaches to treat EV-associated diseases, including vascular calcification and cancer, by suppressing transport of sortilin and disease-causing proteins bound with sortilin to EVs.

### Example 2: Experimental Procedures

### Chemicals and Reams

MG-132 (Cat #7449) and Chloroquine diphosphate salt (Cat #C6628) were purchased from Sigma-Aldrich Co. LLC. (Sigma). 2-fluoropalmitic acid (2-FPA, Cat #90380) was purchased from Cayman Chemical. Sortilin propeptide (sortilin-derived propeptide) (Cat #049-75, Lot #432841) was purchased from Phoenix Pharmaceuticals, Inc. Primers were purchased from Integrated DNA Technologies, Inc. PCR reagents were purchased from EMD Millipore Corporation.

### Vectors and Constructs

Expression vectors were constructed in pcDNA3.1 (+) vector (Thermo Fisher Scientific Inc., Cat #V79020). Constructs of human sortilin (NM 002959.5) were generated by inserting FLAG (DYKDDDDK) or 6×His (HHHHHH) tag into 3 amino acids (S73A1330) behind the furin cleavage site R74WRR77 (35) using site-mutagenesis: pcDNA3.1(+) FLAG-sortilin full-length (Full), amino acids (aa) 1-831; pcDNA3.1(+) FLAG-sortilin ECD+TMD, aa1-778; pcDNA3.1(+) FLAG-sortilin ICD+TMD, aa1-831 (A81-754); pcDNA3.1(+) 6×His-sortilin full-length (Full); pcDNA3.1(+) 6×His-sortilin ECD+TMD; pcDNA3.1(+) 6×His-sortilin ICD+TMD; pcDNA3.1(+) 6×His sortilin 10CC domain+TMD, aa1-778 (681-604); pcDNA3.1(+) FLAG-sortilin C783A; pcDNA3.1(+) 6×His-sortilin ICD+TMD C783A; pcDNA3.1(+) FLAG-sortilin S316E; pcDNA3.1(+) FLAG-sortilin without propeptide, aa1-831 (34-77). Constructs of sortilin CD43-TMD were generated by overlapping PCR strategy using a CD43 expression vector (Origene, Cat #RC204195, NM 003123) (25): pcDNA3.1(+) FLAG-sortilin CD43-TMD, sortilin aa1-754, CD43 aa254-276, sortilin aa779-831; pcDNA3.1(+) 6×His-sortilin CD43-TMD. Expression vector of sortilin with 3×FLAG at C-terminus (sortilin-3×FLAG, Cat #EX-M0397-M14) was purchased from GeneCopoeia, Inc.

### Western Blot Analysis

Cells, EVs and the supernatant of culture medium were lysed with IP lysis buffer (Thermo Fisher Scientific Inc., Cat #87787) containing protease inhibitor (Roche Diagnostics, Cat #04693159001). Protein concentration was measured using the bicinchoninic acid (BCA) method (Thermo Fisher Scientific Inc., Cat #23225). Laemmli buffer (Boston Bioproduct; non-reducing, Cat #BP-110NR; reducing, Cat #BP-111R) was added to the lysate, and boiled at 95° C. for 5 minutes. Total protein was separated by 4-12% SDS-PAGE and transferred to polyvinylidene difluoride (PVDF) membrane using iBlot Western blotting system (Life Technologies) or conventional wet method. Primary antibodies against human sortilin ICD (rabbit 1:1000; Abcam plc., Cat #ab16640, Lot #GR185198-1), human (3-actin (mouse 1:2000; Novus Biologicals, LLC, Cat #NB600-501, Lot #014M4759), FLAG (rabbit 1:1000; Sigma, Cat #F4725, Lot #093M4798, mouse(M2) 1:1000; Sigma, Cat #F1804, Lot #SLBJ4607V), 6×His (mouse 1:1000; Abcam plc., Cat #ab18184, Lot #GR247674-1).

### Immunoprecipitation

Cells or EVs were lysed in IP lysis buffer. Anti-FLAG M2 antibody (5 p.g) or mouse IgG (5 lig, R&D Systems, Cat #MAB002, Lot #IX2415091) were incubated with Dynabeads with Protein G (Thermo Fisher Scientific Inc., Cat #10004D) by rotation for overnight at 4° C. Cell lysate was incubated for 4 hours at 4° C. under rotating conditions. The bead-antibody-protein complex was washed with PBS 3 times. Then, Laemmli buffer was added to the precipitates for SDS-PAGE.

### Cross-Linking Experiment

Chemical cross-linking was carried out by incubating HEK293 cells transiently overexpressing FLAG-sortilin Full, ECD+TMD, or ICD+TMD with 1 mmol/L of bis (sulfosuccinimidyl) suberate (BS3), water-soluble, non-cleavable cross-linker (Thermo Fisher Scientific Inc., Cat #21580) at room temperature for 30 minutes. Reaction was stopped with 15-minute incubation of 1 mol/L Tris-HCl (pH7.4), and cells were centrifuged at 1,000 rpm for 5 minutes to remove the buffer including BS3, and washed with PBS. Then, cells were lysed in IP lysis buffer for western blotting.

### Cell Culture of HEK293 Cells and Establishment of Transfectants

HEK293 cells were purchased from American Type Culture Collection (ATCC) and maintained in Eagle's minimum essential medium (EMEM, ATCC, Cat #30-2003) supplemented with 10% fetal bovine serum (FBS), penicillin (100 U/mL), and streptomycin (100 p.g/mL) at 37° C. in a humidified atmosphere of 5% CO2. For transfection in HEK293 cells, Lipofectamine 2000 reagent (Thermo Fisher Scientific Inc., Cat #11668019) was used, according to the manufacturer's protocol. HEK293 cells stably expressing FLAG-sortilin Full, ECD+TMD, ICD+TMD and sortilin-3×FLAG were obtained by transfection with pcDNA3.1(+) FLAG-sortilin Full, pcDNA3.1(+) FLAG-sortilin-ECD+TMD, pcDNA3.1(+) FLAG-sortilin ICD+TMD, and expression vector of sortilin-3×FLAG (GeneCopoeia, Inc., Cat #EX-M0397-M14), respectively. These cell lines were maintained in EMEM supplemented with 10% FBS, penicillin/streptomycin, and geneticin 800 p.g/mL. Cells were incubated in the incubator with MG-132 and chloroquine during the indicated time, and with 2-FPA and sortilin propeptide for 24 hours, respectively.

### Separation of Culture Medium to Supernatant and EVs

Separation of culture medium to supernatant and EVs was performed according to the protocol previously reported (41). Culture medium underwent centrifugation at 1,000 rpm for 5 minutes to remove cell debris. Then, the supernatant and EVs were separated by ultracentrifugation at 100,000 g for 40 minutes at 4° C. (Optima Max Ultracentrifuge, Beckman Coulter).

### TR-FRET and Homogenous TR-FRET (HTRF)

TR-FRET and HTRF were performed as described (26). FLAG-sortilin Full HEK293 cells were harvested using dissociation solution (e.g., from Sigma Aldrich, Cat #C5914) 24 hours after transfection of 6×His-sortilin expression vector. An incubation under the circle rotator was performed at 4° C. with 1×106 cells/mL for TR-FRET and 2×106 cells/mL for HTRF containing 1 nmol/L Anti-FLAG®(M2)-Cryptate (Cisbio Bioassays, Cat #61FG2KLA, Lot #25A) and 3 nmol/L Anti-6HIS-XL665 (Cisbio Bioassays, Cat #61HISXLA, Lot #56A) in PBS supplemented with 25% FBS. For TR-FRET, cells were centrifuged at 1,000 rpm for 5 min to remove the antibodies, and resuspended in PBS, and applied into a 96-well white plate. For HTRF, cells were applied without removing the antibodies into a 96-well white plate. Then, the plate was read (excitation: 320 nm, emission: 620 nm (cutoff 570 nm), 665 nm (cutoff 630 nm), Delay 50 ps, Integration 500 ps). The FRET signal was calculated as (the ratio of counts per seconds 665:620)×10,000, and % change of the FRET signal by 6×His-sortilin expression was expressed.

### Purification of Soluble Sortilin

EV-deprived culture medium of FLAG-sortilin Full or ECD+TMD HEK293 cells were subjected to ANTI-FLAG® M2 Affinity Gel (Sigma, Cat #A2220). Soluble sortilin with FLAG-tag was eluted with 100 μg/mL FLAG-peptide (Sigma, F3290, Lot #SLBR6767V). Purified soluble sortilin was dialyzed in PBS.

### Statistical Analysis

Data are presented as means±S.E. of the indicated number. Comparison was performed by analysis of variance following unpaired t-test.

### REFERENCES FOR EXAMPLES 1 & 2

1. Raposo, G., and Stoorvogel, W. (2013) Extracellular vesicles: exosomes, microvesicles, and friends. J. Cell Biol. 200, 373-383.
2. Simons, M., and Raposo, G. (2009) Exosomes—vesicular carriers for intercellular communication. Curr. Opin. Cell Biol. 21, 575-581.
3. Thery, C., Ostrowski, M., and Segura, E. (2009) Membrane vesicles as conveyors of immune responses. Nat. Rev. Immunol. 9, 581-593
4. Carlo, A. S., Nykjaer, A., and Willnow, T. E. (2014) Sorting receptor sortilin-a culprit in cardiovascular and neurological diseases. J. Mol. Med. (Berl). 92, 905-911
5. Beraud-Dufour, S., Devader, C., Massa, F., Roulot, M., Coppola, T., and Mazella, J. (2016) Focal Adhesion Kinase-Dependent Role of the Soluble Form of Neurotensin Receptor-3/Sortilin in Colorectal Cancer Cell Dissociation. Int. J. Mol. Sci. 17, 1860
6. Wilson, C. M., Naves, T., Vincent, F., Melloni, B., Bonnaud, F., Lalloue, F., and Jauberteau, M. O. (2014) Sortilin mediates the release and transfer of exosomes in concert with two tyrosine kinase receptors. J. Cell. Sci. 127, 3983-3997
7. Wilson, C. M., Naves, T., Akhrass H. A., Vincent, F., Melloni, B., Bonnaud, F., Lalloue, F., and Jauberteau, M. O. (2016) A new role under sortilin's belt in cancer. Commun. lntegr. Biol. 9, e1130192
8. Roselli, S., Pundavela, J., Demont, Y., Faulkner, S., Keene, S., Attia, J., Jiang, C. C., Zhang, X. D., Walker, M. M., and Hondermarck, H. (2015) Sortilin is associated with breast cancer aggressiveness and contributes to tumor cell adhesion and invasion. Oncotarget 6, 10473-10486
9. Kaddai, V., Jager, J., Gonzalez, T., Najem-Lendom, R., Bonnafous, S., Tran, A., Le
10. MarchandBrustel, Y., Gual, P., Tanti, J. F., and Cormont, M. (2009) Involvement of TNF-alpha in abnormal adipocyte and muscle sortilin expression in obese mice and humans. Diabetologia 52, 932-940
11. Gustafsen, C., Kjolby, M., Nyegaard, M., Mattheisen, M., Lundhede, J., Buttensch0n, H., Mors, O., Bentzon, J. F., Madsen, P., Nykjaer, A., and Glerup, S. (2014) The hypercholesterolemia-risk gene SORT1 facilitates PCSK9 secretion. Cell Metab. 19, 310-318
12. Kjolby, M., Andersen, O. M., Breiderhoff, T., Fjorback, A. W., Pedersen, K. M., Madsen, P., Jansen, P., Heeren, J., Willnow, T. E., and Nykjaer, A. (2010) Sort1, encoded by the cardiovascular risk locus 1p13.3, is a regulator of hepatic lipoprotein export. Cell Metab. 12, 213-223
13. Musunuru, K., Strong, A., Frank-Kamenetsky, M. et al. (2010) From noncoding variant to phenotype via SORT 1 at the 1p13 cholesterol locus. Nature 466, 714-719
14. Patel, K. M., Strong, A., Tohyama, J., Jin, X., Morales, C. R., Billheimer, J., Millar, J., Kruth, H., and Rader, D. J. (2015) Macrophage sortilin promotes LDL uptake, foam cell formation, and atherosclerosis. Circ. Res. 116,789-796.
15. Mortensen, M. B., Kjolby, M., Gunnersen, S., Larsen, J. V., Palmfeldt, J., Falk, E., Nykjaer, A., and Bentzon, J. F. (2014) Targeting sortilin in immune cells reduces proinflammatory cytokines and atherosclerosis. J. Clin. Invest. 124, 5317-5322
16. Hu, F., Padukkavidana, T., Vwgter, C. B., Brady, O. A., Zheng, Y., Mackenzie, I. R., Feldman, H. H., Nykjaer, A., and Strittmatter, S. M. (2010) Sortilin-mediated endocytosis determines levels of the frontotemporal dementia protein, progranulin. Neuron 68, 654-667
17. Carrasquillo, M. M., Nicholson, A. M., Finch, N. et al. (2010) Genome-wide screen identifies rs646776 near sortilin as a regulator of progranulin levels in human plasma. Am. J. Hum. Genet. 87, 890-897
18. Carlo, A. S., Gustafsen, C., Mastrobuoni, G., Nielsen, M. S., Burgert, T., Hartl, D., Rohe, M., Nykjaer, A., Herz, J., Heeren, J., Kempa, S., Petersen, C. M., and Willnow, T. E. (2013) The pro-neurotrophin receptor sortilin is a major neuronal apolipoprotein E receptor for catabolism of amyloid-F3 peptide in the brain. J. Neurosci. 33, 358-370
19. Andersson, C. H., Hansson, O., Minthon, L., Andreasen, N., Blennow, K., Zetterberg, H., Skoog, I., Wallin, A., Nilsson, S., and Kettunen, P. (2016) A Genetic Variant of the Sortilin 1 Gene is Associated with Reduced Risk of Alzheimer's Disease. J. Alzheimers Dis. 53, 1353-1363
20. Goettsch, C., Hutcheson, J. D., Aikawa, M., Iwata, H., Pham, T., Nykjaer, A., Kjolby, M., Rogers, M., Michel, T., Shibasaki, M., Hagita, S., Kramann, R., Rader, D. J., Libby, P., Singh, S. A., and Aikawa, E. (2016) Sortilin mediates vascular calcification via its recruitment into extracellular vesicles. J. Clin. Invest. 126, 1323-1336
21. Dumaresq-Doiron, K., Jules, F., and Lefrancois, S. (2013) Sortilin turnover is mediated by ubiquitination. Biochem. Biophys. Res. Commun. 433, 90-95
22. Li, J., Bi, L., Hulke, M., Li, T. (2014) Fish oil and fenofibrate prevented phosphorylation-dependent hepatic sortilin 1 degradation in Western diet-fed mice. J. Biol. Chem. 289, 22437-22449
23. McCormick, P. J., Dunnaresq-Doiron, K., Pluviose, A. S., Pichette, V., Tosato, G., and Lefrancois, S. (2008) Palmitoylation controls recycling in lysosomal sorting and trafficking. Traffic 9, 1984-1997
24. Ward, R. J., Xu, T. R., Milligan, G. (2013) GPCR oligomerization and receptor trafficking. Methods Enzymol. 521, 69-90
25. Eggert, S., Gonzalez, A. C., Thomas, C., Schilling, S., Schwarz, S. M., Tischer, C., Adam, V., Strecker, P., Schmidt, V., Willnow, T. E., Hermey, G., Pietrzik, C. U., Koo, E. H., and Kins, S. (Aug. 10, 2017) Dimerization leads to changes in APP (amyloid precursor protein) trafficking mediated by LRP1 and SorLA. Cell. Mol. Life Sci. 10.1007/s00018-017-2625-7
26. Miner, J. J., Shao, B., Wang, Y., Chichili, G. R., Liu, Z., Klopocki, A. G., Yago, T., McDaniel, J. M., Rodgers, W., Xia, L., and McEver, R. P. (2011) Cytoplasmic domain of P-selectin glycoprotein ligand-1 facilitates dimerization and export from the endoplasmic reticulum. J. Biol. Chem. 286, 9577-9586
27. Maurel, D., Kniazeff, J., Mathis, G., Trinquet, E., Pin, J. P., and Ansanay, H. (2004) Cell surface detection of membrane protein interaction with homogeneous time-resolved fluorescence resonance energy transfer technology. Anal. Biochem. 329, 253-262
28. Westergaard, U. B., Sorensen, E. S., Hermey, G., Nielsen, M. S., Nykjaer, A., Kirkegaard, K., Jacobsen, C., Gliemann, J., Madsen, P., and Petersen, C. M. (2004) Functional organization of the sortilin VpslOp domain. J. Biol. Chem. 279, 50221-50229
29. Kitago, Y., Nagae, M., Nakata, Z., Yagi-Utsumi, M., Takagi-Niidome, S., Mihara, E., Nogi, T., Kato, K., and Takagi, J. (2015) Structural basis for amyloidogenic peptide recognition by sorLA. Nat. Struct. Mol. Biol. 22, 199-206
30. Quistgaard, E. M., Madsen, P., GrOftehauge, M. K., Nissen, P., Petersen, C. M., and Thirup, S. S. (2009) Ligands bind to Sortilin in the tunnel of a ten-bladed beta-propeller domain. Nat. Struct. Mol. Biol. 16, 96-98
31. Goettsch, C., Iwata, H., Hutcheson, J. D., O'Donnell, C. J., Chapurlat, R., Cook, N. R.,
Aikawa, M., Szulc, P., and Aikawa, E. (2017) Serum Sortilin Associates With Aortic Calcification and Cardiovascular Risk in Men. Arterioscler. Thromb. Vasc. Biol. 37, 1005-1011
32. Ogawa, K., Ueno, T., Iwasaki, T., Kujiraoka, T., Ishihara, M., Kunimoto, S., Takayama, T., Kanai, T., Hirayama, A., and Hattori, H. (2016) Soluble sortilin is released by activated platelets and its circulating levels are associated with cardiovascular risk factors. Atherosclerosis 249, 110-115
33. Buttenschon, H. N., Demontis, D., Kaas, M., Elfving, B., MOlgaard, S., Gustafsen, C., Kaerlev, L., Petersen, C. M., BOrglum, A. D., Mors, O., and Glerup, S. (2015) Increased serum levels of sortilin are associated with depression and correlated with BDNF and VEGF. Trans. Psychiatry 5, e677
34. Massa, F., Devader, C., Beraud-Dufour, S., Brau, F., Coppola, T., and Mazella, J. (2013) Focal adhesion kinase dependent activation of the P13 kinase pathway by the functional soluble form of neurotensin receptor-3 in HT29 cells. Int. J. Biochem. Cell Biol. 45, 952-959
35. Nyborg, A. C., Ladd, T. B., Zwizinski, C. W., Lah, J. J., and Golde, T. E. (2006) Sortilin, SorCS1b, and SorLA Vps10p sorting receptors, are novel gamma-secretase substrates. Mol. Neurodegener. 1, 3
36. Kienlen-Campard, P., Tasiaux, B., Van Flees, J., Li, M., Huysseune, S., Sato, T., Fei, J. Z., Aimoto, S., Courtoy, P. J., Smith, S. O., Constantinescu, S. N., and Octave, J. N. (2008) Amyloidogenic processing but not amyloid precursor protein (APP) intracellular C-terminal domain production requires a precisely oriented APP dimer assembled by transmembrane GXXXG motifs. J. Biol. Chem. 283, 7733-7744
37. Mazella, J., Zsurger, N., Navarro, V., Chabry, J., Kaghad, M., Caput, D., Ferrara, P., Vita, N., Gully, D., Maffrand, J. P., and Vincent, J. P. (1998) The 100-kDa neurotensin receptor is gp95/sortilin, a non-G-protein-coupled receptor. J. Biol. Chem. 273, 26273-26276
38. Massa, F., Devader, C., Lacas-Gervais, S., Beraud-Dufour, S., Coppola, T., and Mazella, J. (2014) Impairement of HT29 Cancer Cells Cohesion by the Soluble Form of Neurotensin Receptor-3. Genes Cancer 5, 240-249
39. Lara-Castro, C., Luo, N., Wallace, P., Klein, R. L., and Garvey, W. T. (2006) Adiponectin multimeric complexes and the metabolic syndrome trait cluster. Diabetes 55, 249-259
40. Waki, H., Yamauchi, T., Kamon, J., Ito, Y., Uchida, S., Kita, S., Hara, K., Hada, Y., Vasseur, F., Froguel, P., Kimura, S., Nagai, R., and Kadowaki, T. (2003) Impaired multimerization of human adiponectin mutants associated with diabetes. Molecular structure and multimer formation of adiponectin. J. Biol. Chem. 278, 40352-40363
41. Li, W., Li, C., Zhou, T., Liu, X., Liu, X., Li, X., Chen, D. (2017) Role of exosomal proteins in cancer diagnosis. Mol Cancer 16, 145
42. Hutcheson, J. D., Goettsch, C., Pham, T., lwashita, M., Aikawa, M., Singh, S. A., and Aikawa, E. (2014) Enrichment of calcifying extracellular vesicles using density-based ultracentrifugation protocol. J. Extracell. Vesicles. 3, 25129.

### Example 3: Sortilin Dimerization can Increase Calcification in Human Coronary Artery Smooth Muscle Cells

Extracellular vesicles (EVs) play a critical role in intercellular communication and are involved in various diseases. Sortilin promotes vascular calcification via its trafficking of tissue-nonspecific alkaline phosphatase, a facilitator of calcification, to EVs. Sortilin is synthesized with a propeptide, which is cleaved in the late Golgi network to generate the mature form. The propeptide exhibits high affinity to mature sortilin and hinders ligand binding. In HEK293 cell line, sortilin dimerization was decreased by propeptide treatment, and the sortilin mutation (S316E), which inhibits propeptide binding, increased sortilin dimerization. These results suggested that sortilin dimerization have an important role in sortilin trafficking (Itoh S et al, JBC, 2018). Moreover, sortilin forms an intermolecular disulfide bond at cysteine 783 site, and loss of the intermolecular disulfide bond by mutation (C783A) cease the transport of dimerized sortilin to EVs. To further understand the role of sortilin in vascular calcification, the effects of S316E and C783A mutations of sortilin on the dimer and multimer formation in primary human coronary artery smooth muscle cells (HCASMCs), a major source of vascular calcification, was examined. Sortilin fused with His- or Flag-tag proteins were induced by adenovirus infection, and then dimer and/or multimer of sortilin in HCASMCs and HCASMCs-derived EVs were detected using non-reducing western blotting, which maintained disulfide bounds. As a result, the S316E mutant showed an increase of multimer in HCASMCs and dimer in EVs. These results indicate that an increase of sortilin trafficking to EVs, can promote calcification. Furthermore, C783A mutant showed the decrease of sortilin dimerization in HCASMCs and EVs, suggesting that a decrease of sortilin trafficking to EVs can also occur. In conclusion, formation of dimers and multimers is fundamental for sortilin trafficking in HCASMCs-cells and HCASMC-derived EVs. Inhibition of sortilin dimerization in HCASMCs and HCASMC-derived EVs can further prevent vascular calcification.

### Results

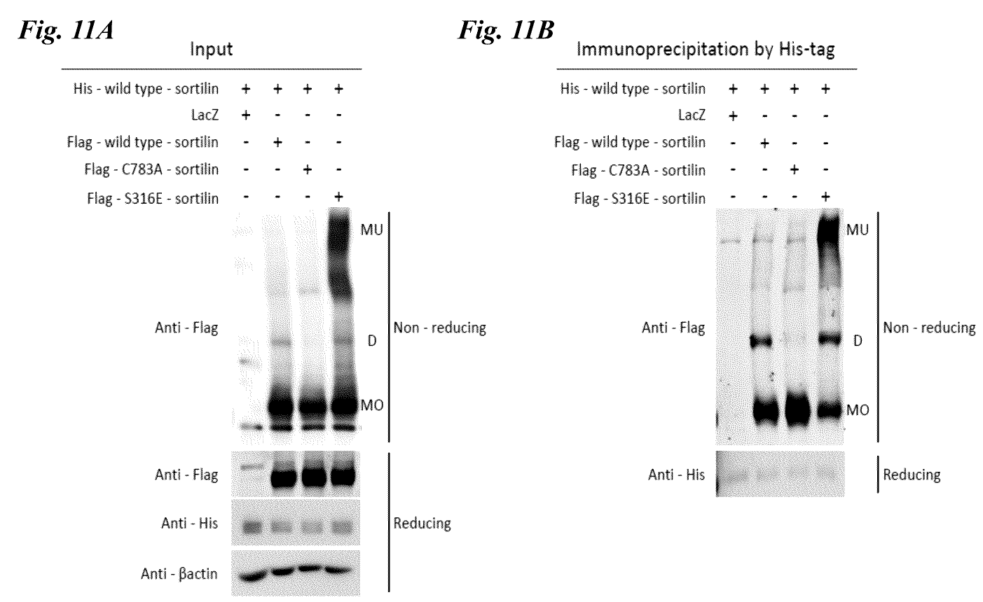

HCASMCs expressing His-wild type sortilin were infected with LacZ, Flag-wild type sortilin, Flag-C783A mutant sortilin and Flag-S316E mutant sortilin. HCASMCs were lysed 3 days after infection. Sortilin expression was detected by the non-reducing or reducing (cleavage disulfide bounds) western blotting in input sample (FIG. 11A) or immunoprecipitated sample (FIG. 11B). The results show that C783A mutant sortilin decreases dimer formation and S316E mutant increases multimer formation in HCASMC lysates (FIG. 11A-B).

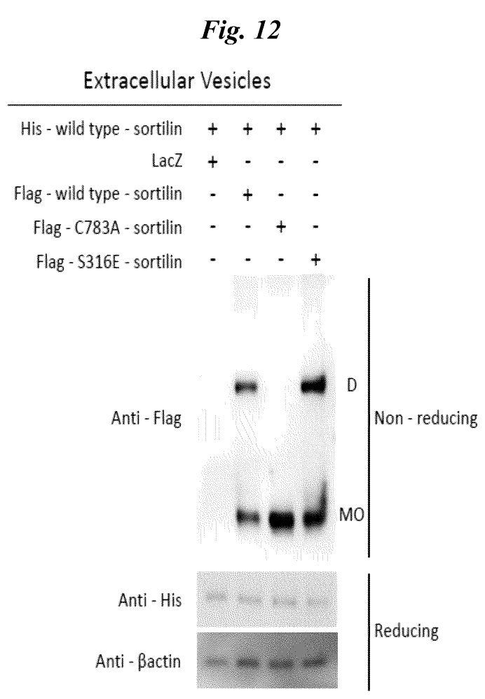

HCASMCs expressing His-wild type sortilin were infected with LacZ, Flag-wild type sortilin, Flag-C783A mutant sortilin and Flag-S316E mutant sortilin. HCASMCs-derived EVs were isolated from culture medium 3 days after infection. Sortilin expression was detected by the non-reducing or reducing western blotting. The results show that C783A mutant sortilin decreases dimer formation and S316E mutant increases dimer formation in HCASMCs-derived EVs (FIG. 12).

### HCASMCs Culture and Adenovirus Infection

HCASMCs (PromoCell) were grown in SMC growth medium 2 (SMC-GM2, PromoCell) supplemented with epidermal growth factor (0.5 ng/ml), insulin (5 μg/ml), basic fibroblast growth factor-B (2 ng/ml) and FBS (5%). HCASMCs from 3 independent donors were used and all assays were performed at passages 8. HCASMCs were transduced with His-tagged wild type sortilin and LacZ, Flag-tagged wild type, C783A or S316E sortilin adenoviruses at MOI (multiplicity of infection) of 1000.

### Methods

Immunoprecipitation: HCASMCs were lysed with IP lysis buffer (Thermo Scientific) containing protease inhibitor (Roche). Cell lysates were incubated with Dynabeads His-Tag Isolation & Pulldown (Thermo Scientific) for 4 hours at 4° C. under rotating conditions. The bead-protein complexes were washed 3 times with IP lysis buffer. Then, precipitates were dissolved by laemmli buffer (Boston Bioproduct) for SDS-PAGE.

Isolation of HCASMCs-derived EV from culture medium: Culture medium underwent centrifugation at 1,000 rpm for 5 minutes to remove cell debris. Then, the supernatant and EVs were separated by ultracentrifugation at 100,000 g for 60 minutes at 4° C. (Beckman Coulter). EVs were lysed by IP lysis buffer.

Western blot analysis: Protein was separated by SDS-PAGE and transferred by conventional wet method. Primary antibodies against Flag (Sigma, F7425), His (MBL, JM-3646) and human β-actin (Novus, #AC-15) were used. β-actin was used as loading control. Protein expression was detected using Pierce ECL Western Blotting Substrate Reagent (Thermo Scientific) and ImageQuant LAS 4000 (GE Healthcare).

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### SEQUENCES

SEQ ID NO: 1 (Sortilin full sequence-Cys⁷⁸³):
MERPWGAADG LSRWPHGLGL LLLLQLLPPS TLSQDRLDAP
PPPAAPLPRW SGPIGVSWGL RAAAAGGAFP RGGRWRRSAP
GEDEECGRVR DFVAKLANNT HQHVFDDLRG SVSLSWVGDS
TGVILVLTTF HVPLVIMTFG QSKLYRSEDY GKNFKDITDL
INNTFIRTEF GMAIGPENSG KVVLTAEVSG GSRGGRIFRS
SDFAKNFVQT DLPFHPLTQM MYSPQNSDYL LALSTENGLW
VSKNFGGKWE EIHKAVCLAK WGSDNTIFFT TYANGSCKAD
LGALELWRTS DLGKSFKTIG VKIYSFGLGG RFLFASVMAD
KDTTRRIHVS TDQGDTWSMA QLPSVGQEQF YSILAANDDM
VFMHVDEPGD TGFGTIFTSD DRGIVYSKSL DRHLYTTTGG
ETDFTNVTSL

1. A method for inhibiting or reducing trafficking of sortilin to an extracellular vesicle (EV) from a cell, the method comprising inhibiting covalent intermolecular dimerization of sortilin in the cell.

2. The method of claim 1, wherein said inhibiting comprises inhibiting formation of an intermolecular disulfide formation at Cys⁷⁸³ of SEQ ID NO: 1.

3. The method of claim 1, wherein said inhibiting comprises administering a peptide to the cell.

4. The method of claim 3, wherein said peptide is a sortilin-derived propeptide comprising an amino acid sequence of SEQ ID NO: 2.

5. The method of claim 3, wherein the peptide comprises at least one modification.

6. The method of claim 3, wherein the peptide is amidated, acetylated, cyclized, phosphorylated, glycosylated, nitrosylated, methylated, lipidated, or PEGylated.

7. The method of claim 3, wherein the peptide comprises at least one D amino acid, beta amino acid or modified peptide linkage.

8. The method of claim 3, wherein the peptide comprises at least one substituted amino acid.

9. The method of claim 1, wherein the cell is a leukocyte, lymphocyte, macrophage, natural killer cell, dendritic cell, T cell, or B cell.

10. The method of claim 1, wherein said inhibiting is in in vitro or ex vivo.

11. The method of claim 1, wherein said inhibiting is in vivo.

12. The method of claim 11, wherein said inhibiting is in a mammal.

13. The method of claim 11, wherein said inhibiting is in a subject having or suspected of having an extracellular vesicle associated disease (EV-associated disease).

14. The method of claim 13, wherein the EV-associated disease is selected from the group consisting of: calcific aortic valve disease, diabetes, systemic lupus erythematosus, ulcerative colitis, pulmonary fibrosis, nan-alcoholic fatty liver disease, osteoporosis, Alzheimer's disease, scleroderma, atherosclerosis, myocardial infarction, hypercholesterolemia, cancer, rheumatoid arthritis, and obesity.

15. The method of claim 14, where diabetes is type 1 diabetes, type 2 diabetes, or maturity onset diabetes in the young.

16. The method of claim 14, wherein cancer is lung cancer or pancreatic cancer.

17-38. (canceled)


SEQUENCE LISTING (6 sequences)

SEQ ID NO: 1 - protein, 831 aa, Homo sapiens [sequence omitted]

SEQ ID NO: 2 - protein, 44 aa, Homo sapiens
QDRLDAPPPPAAPLPRWSGPIGVSWGLRAAAAGGAFPRGGRWRR

SEQ ID NO: 3 - DNA, 95377 bp, Homo sapiens
  source 1..95377: mol_type = genomic DNA
cctgaggttgggagttcgagaccagcctgaccaacatggagaatcgccgtctctactaaaaatacaaaattagccgggtgtggtggcgcatgtctgtaatcccagctactcgagaggctgaggcaggagaatcgcttgaacccgggaggcggaggttgcggtgagcctagatcacgccattgcactccagcctgaacaacaagagcgaaactccatctcaaaacaaaaccaaaaacaaacaaaaaaaacttttttgttaagttttgccacataaacaattttagagaaaatacctgacaatagttaatgattatttgttgtaggcaataatggtctaagcactttatatatattaacatttaatcctcacaactactctatgaattagctattcttattgtcctcattgtacagatgaagaaatagaggcacagagtggccaagtaacctcaagtcacacacacaagctcaacttttaagtatttaaatatacttcaaatatatttctaaaggagtctttaacagaaaaaagtactgactccatgtactttgctgctgcttttgtgatccctgttgaagtgacttcaagaagtgacaatgatgccaggcatttggttaagtatgatgtggtgagttaaccttaaaatagaaaatttggtcaataagcatttttattttggagccagagagctattttagggacctgctgcctggaaaggatatccttgggacagcagatcccttagttcctaatgaatagctgcatcaccaacacagcttctcctgaccatggtagggccatcagcagggtaactcagcccagcttattaacccttctgttttctttccagctagccacagtgcagcctggccagaatttccacatgttcacaaaggaagaacttgaagaggttatcaaggacatttaaggaatcctgatcctcagaacttctctgggacaatttcagttctaataatgtccttaaattttatttccagctcctgttccttggaaaatctccattgtatgtgcattttttaaatgatgtctgtacataaaggcagttctgaaataaagaaaattttaaaatatttgttaatagactgttctcttctaatagtcttttttttttttttttttttaagagatgaggtctcactatattgcccaggctggtttcaaactcctgggttcaagtgatcttcccgcctctgcctcctgaagtgctaggattataggcgagagccactgtgcccagccattgtaatacagtcttttgtttgaaatgcaaatgttagtgggccaaaaggacagtgttattaatatccctattctatgaagatgtctgttctaaaactgttttagtgatgctctataacagaatcaactatatccattaatcccatggtgttgataaatgaactagtccagctcttcccagtggttaatagatgttaacagcactgtaaatccatttcggccctataatttctggagcaaacgtgtatttttgctgcttttcttataaggagatgctactaattgactatcagaagaacatgttttgaggtcctcctcaaaacattgatgggttggaatcagccatcaacagtgttgtaccagttcttgggtcttgatagacaatttggaaagagatttccttgtttacgaagtgaaggactaagaattaaattaatgaccttggcctcactggcaatcaattacaaatctctatatcagtaagagaattgtaaaattcagaagcagtttacattagactttggaatgaatgatgaactttccccattagagctgccatcatatgattggtggagattgtttaaagaatcattctatctcagcctcaaaaattaccacgggaagatggtatcagtatcactacttaacagatgaagaatctaatgctgtgtgcccatggacacacaaaattaagctgttgggacttgaacagctcaaattttcttgtttcagatcaccagctcttgagtaatagggaaatctggagatttgaaaagtacttgcactgtccacaacatggctaatccatatgtggatgatgcagaattgatttaattcactaacagggaccccataattatttgctgggctctgggtatattgatgtgtaggaagttgtactatatgttaataaattatgactatttggataggctgaattcatactaaatccatactgtagttcaacaaaaaacaacatgtttatatacatttggaaactgcaatgataggaaacatttggaagaaagggattttgccctagaataacacataaggaaagcagagaattagagtatattgttactgaatgttccagagacttctagtgattttaacacttattaagtatagcatgttgatggggagatttgtttctctaaagatcactttgttttattaaatcctaatagaaaatacccttgaaaatcctatctcatttattcctgtaacaaatttattgagcacctacctactatatgccaagaaatgtattagccatcaggcagagagctgttcaaatggtagatatgatctgtgctcctgtgaaacttctagtggggagagccaaacaataaacaatattcctttttaactctaggttaatgcagaaatggataaagcccatgttgcttttgggagaggtgagttgtgtctctgaatcacatgcatttcatagggggaaaaatgcccaaatactaaggagtagctcttagcctatgtccgttcgcctcagcttaagttgttttccctcccactggctgggcagcatatttgtgttttacctggtaaaacagtaaatttcgtaccttctataattggcattcttcaaagaggtagaactttgatttttttgtagaatattaaaacaagctttcttaggttaaagaagtgatcttactacgaacagcaatggtttcatgatgtgtaattgctttatgttatctttctgctatagaatgtttcctggagaaaggtacggtttttagtaataagataaatttactttagtcctgcaaataaggtgaaatcttatgtccagtatctcacaaaggagaactgacagtgccacttttatatttaatgtcaatctcattagacaaaaatgaaatatagttcctagggtttccaatttaaaaagtgaaataataaatatattcactcaaacattttctcagtgccatatgcaagttaccatgtcccagaagctgatacagtttgaagaaggaaatatataagtactgtaagcctgtgataactaaaagcaacatactaaaaagtgaaggtacagataattggaacaaatttaatagcgtttaaaagtgtatttggtaatttctccaggaggtggcactgttgagttgttttttcttttcagacagtaagacaatagaccattcatctttgtgtcacccaaacaaagaaatattcctttataaccccctttcctccccccacctaatcacatgctactggaactgactgtcttgtgatctactcccatagttggcatagatttaaaaataatcttttttaaattaatctgatagtagttatggaaaggaacaaattgttcaccaattttcatcagctttatcttttcagtccagtaactgagaacttaaaaagactattctagtgattttactatcctgctcagatggcattcaaaaacatttttcacttctcacgtcttggaaaaatttgtaaattcgttaagtctttttgtttgaaagttaatgtgactggaaatattttaatgtaaatatcgggttgggtgcattgtatcattttgctctacaatgactatatcctcatttgggtttatggccagtttataattaaaagtaatctttacttgctttgagtgtaatgacttgcatattttgtatctttctatttgctaccttttctactgaacaatttctagctttctgaagggatggagtcagtaagcttaaggattcagaactggttttacctgtgctacgtgttgtagaagttgtgtgtgcagcttctgcggtaaagggaacagcgtaggggttttattttattcattagaggtggatcggggtgctaatttagaggacatcacatactggaaccaaacttgtccgaagctctcactgcagagttaaggcactttggtaaaataaatgtgcatctaggaaatgatcatgtgtacagtaggcattggatcattctcttgcccctcttttacctgcccctaatgcctaagctacattcgaagtcctggaaaacctgtaagtaattccgcctttggcctacctcccctcagaagaggaaggtgagagaggccaaacgtatggaaagactattcttcaggcttgcaggagtcacggatgtcctgatgtctgtctccgtaggatgtggcccacatcagttcgcattcaccttcttgcagcaaggcagctgcttagcagacaatgggcgctccagtgagcggggtttataaaaacccgaagccccggttcatgatggagcccccttttccagctgagcaagctcagggatttcctgggtaggtttttccaggctctgcctcaccgaaggaattttaggagtgtctctggggaacaggagggacgtaacccagccccaacttgagggcgctagaggtgcggcaaggggtcgcgacgccaggagcccggggctcggcgggaaggtatgagaagctcctacgtgaactccacaagccgggcccgggagacgccgggcgaggcggggttgacctcagcagtctctgccccgttccagccaatcagtcccgcatcttagcatccgaatccaggacccccgaagccggaggcgacgcgagccaatgaggagtgggccggggaagagggacaggcggccagcctatggggcggagaggcccggccgcgcgtatccaaggagcgccgcgctcggctcgggggtgtggcgcgcgccggcgggggtgggcgggcgcgccgggcggcaggtgtcggcgtcggcggcattcggcggcgatggagcggccctggggagctgcggacggcctctcgcgctggccccatggcctcggcctcctcctcctcctgcagctgctgccgccgtcgaccctcagccaggaccggctggacgcgccgccgccgcccgctgcgccgctgccgcgctggtctggccccatcggggtgagctgggggctgcgggcggccgcagccgggggcgcgtttccccgcggcggccgttggcgtcgcagcgcgccgggcgaggacgaggagtgcggccgggtccgggacttcgtcgccaagctggccaacaacacgcaccaggtgagcgggcgccgggcccgggagccgctcgggtgcgaggtgccgcccgcgtgcccctcccggcccgtgcgccccgcgactcccggaggagaccccagccctgccgcgcccgcgtcccccgggccgcagccccgaaactgcggcgggcgggcgggcggagtcgggcccgggcggccggcggggctgtgttttgtcataggggaggcaatcttagacttccaccgcattcttttttttttttttaagttgtcaaaatctcttgaacatcgtttttcgtctgtataaataatcaggacccagtaacgctgttaggccccaggaaggtgaaggagtaattaactcttgagcgcttgcagaagttgggctgcgcagaagcaaaggtgtgtcctctctgggtcagtactttccgggtgggagaggcgggagttcaggaggcccgaagcagcgatctggtcaccgtgttttaatagttaaggggactagcttggaaatgagatggcaatatttatgtgatctcttttactgcttcagatttgaccttctgtcatggtacggaagtctgactagcatgtctatatcccgtggtgacaagattttcaatagttaagaaacgcattgcacatattctgctttgagccagctcttggtcaaaagactttcaaaattatgggatttctgtccctggggacaagcagctcaacctttgcacactactggactgacaaaactaattattaagaaagtcctggttccttgcttcagcacaacttgggcatctttctcctatttctctaaaattaacaatttaatccttgctagccacatttgcataatgtattttcaattctatatattttctcccttgcctgttttcctctttcaaagaaaatgaattgcacttgggggcagagatgggtggccaaggctctgtgccagcacttgtccttctgtaaccgtttttcagcgtcagccaagcctcagtctccctttggactgcctggttatctggaccatacttgttacctgtaatagaattctctgatctgaggacacaagacacaaatattcaggaataattgtgcttagtgtatcccgagcagcagctgtgtaaaggtagccaagctagtgctggagggtgagacaggttgggtagaatgagtactgggaagtcttcctacttgatgagaaaaaaaaatcgaatagacttccttctcacagtgggggattgagaaaggctttacaacactaatgcttaatttatactggaccacttccttccttgagctagctggtcacagcattcttgctccatatttcaaattcttctctcgctggttgacctaactttaggtctgctgctactcctggatttagatttactcctgtttttctttttctctttttttgtgagatagagtcttacctcagcctcctgagtagctgggactacaggtgtgcaccactatgcctagctaatttttgtgtaattactattttttttttgagatagggtctcactctgttgcccaggctggaatgcagtggtgtgatcacagctcactgcagccctgacctcctgggctcaagtgatcctctcacctcagcctcccaagtagccgggactactggtacatgcacccagctaattgttcttttctttctttctttctttttttaagaaacagggttttgccatgttccccaggctggtcttgaactcctgggctcaagtgctccacccaccttagcctgccacagtgctgagattagaggcgtgagccactatgccctgcctggatttattccttgagtcagagtcttcctggttagtttgaattgatgtgtcatttgtaatatattgattccaaagaaagaggttgaaaaaactggctcaggggttctaaggccaaatctgtggaagagtagacctaaaaccactcattctttgaagtccacatgtaaagaaattttcttctatttctttcatttagctgtttgcaataatttttttttagtttttctctcccagtctttttttcagtgaccttatttaaaatagctattttggctgggcacggtggctcatgcctgtaatcttatcaatgtgggaggccaaggcaggtggatcacttgaggtcagaagttcaagactagcctggccaacatggtgaaaccctgtctctactaaaaatacaaaagttagccagacgcggtggcatgcgcctgtaattccagttattctggaggctgaggcatgacgatcacttgaacccaggaggcagaggttgcagcgagccgagatcgcaccgttgtactccagcctgggtgacagagtgagactctatctcaaaaaaaaaaaaaaaaaaaaaaaaaaaaaagtttttgcgtttgttagtcatccgtggtctcttcttcagagatctcatcctttctgtatctggttaaaaaaaaaaaaagtggagcctttgtttaatatcatttaccttaaaaatggttaagtagtacttcttaaccaagaattgttatgtagtagcctgttcttctgtttatttatttggttttagaagtcataaaaaatcctgaactgtattgaagagatctagtatttttggaccgtttataaaacagtgtgaaataaaagcatcagcatgacagaacaactttttatcagagatattgtactttgtacaactggggaaggaaggtacatgtagaatcatttttcctacataacatgacaggattcgagtcccaagaaaaagcaaaagttcatggttctcttatttcttttaacttggtcctgtttcatttcaagccacattaattcacatcatcaatgtatgtgaatgccctccaaatgcctagatctgtgctagatgctggggtgccaagatacaaagaaaagatcattcagactggagaatgaaaagcctcagaaggttatagcaaactactagacctgtgtctgtggcttatgaggatatattagcatttaacctgtcttctcaagaaagcacggtaaagctaaagcataatagtgcgtttatattacggtttaaggagaccggtgtcctgatgaccactcaggcttctctgtccatgaaatccagcaggtttttctctttgcccaagcctccctttttctctctaaggttttttttctcatatcatttcttttcaatctattaatttaaaatatatttaaagtgcattgggtttattacacacagcatatattttttcacatagaagtcctaatgattaagtgctttattttaggagttgaggcaggagaggaaaacattttcctttctagttacagtattacaagttagctataatagactcatgggaaagtggaaataaaaaatactggctattactaaaaaaacaaacaaaagggatgacaagaaaacgaaggagaaagataaatagttataactttccaaaaattatgctccctaattttgttctaagggtttgacttaccatggtaaggtggtatagcactgccttatctggactgcctgcatttgaaccctggccatgtcactgtgtgatcctgggtgagttacttaagttttctgtgtctcagttcctaatctgtaaagtgaagataagagcacttaatttgtagggtggttgtgagaattaaatgagtgtgtgttaaattatatatacataatatatacgtatatgtatattattgtataaatatatatttgtattacataatatatgcatatgtaatattgtatttgtaatgagttatagtatgtattacaacatatcacattgtgttgtataatctatacatatgtaataactcattagaagaatgtctggcacataattagcactcagcaattagctatcatttataaatgctctcttcttagactcaataatcttccttttgcctggaactcaaaactgttttcatgccacatttttaactgattctttccttcagctgcttggagtattcatgacatgtataacctttgtacatgcgtgaaattgagcaggcctagaagactttattgttggttactaagttggattgcttcaaaacatttctgctgtaagcacccctttgagggtaggcatgaaactgtcttaaaggaacagatgaaatgccttggccagagttgtggtggtggctgacataatttccttacttctcattcctgtgtacagaccatttgacaatttccttaaaactttttaaacttatgatgaataaagccaggaactcttgcttattttctgtcctccctctgctcctgtcactaactacttctgtaaacataacttcccaacttctactccaactttaaagcatctggttccttaaataaacaggccctgtagagaggccgtgttgtatggggtgtcagctctgggaagaatgcgtgtgtttgtttgctggctctggcagggttgagtccaagctgacaggactgagtgtgcctgtcagaggcttcagggagctgccgctgaagacctcggcctgttgttgtgaatcgccagacctttgtaacacccggaagtctgaaggtcagtcctttaatgccccggaacttcctaactgcctcatttcttccaatacttggcctctcttcatcctaccttccctagcacccccagccgtctagcttatttaatagtctcttgagttctgtttcttccattcagctttggatcaaggagtaggaactctgtaggatctccttcttggtcctggttgagcagagtgagcgaaaccttcatgaagagtcttctctcatgcaatgtatagtcagacctttagcccctagagggatgttaaaatgacttatgcattggatatttcagtaagaagtatcagcagatttgatgaagtgtaatttatctggggaaaggttagtcttcatctgaagtttggttgggatgagagtagaaagctagccaggtatgacagatggctccaagggaatgcagtgttaacaattaggtaagttctctgctgactcaaatgtttggctctgtgcacagtgatttttaattagaaatctgagtctcatgtctgcttgtggaaataattggaagaaggcttatgttctgttcaatttctaatggctattttctttttgtgattattaacaacaataataggctttctatcctgctgttttgatcagatctttcatgtcagggtcacagatactattagcagtttcttctgcactctgccagtttccagtcaactctcgtcaacaggcattgggttctcagtgggtgactgcagagtctgcgttccccaactttgtcccagttggcctgttacccttgtgtgaccatcacatgctgggcagtgcttagcaccaagttgtaatggtcaggacgggggagtaacgtttgttcttctccctccttcctacctaatcctacccctcaattttttttacagaaagtgaactatgttttgaggctgcagaaatgaggctcagatattttatataattaggagctaatattaaggcagacaaagccatcacatttgacatgtaatttaaaatacagcttttctagaaacaacaaaactgtggcaatggctctgacaatattatgaactagatactgcatttgtagatttctacaagtttcagttccctttctctccttttgtcatcagtgtttttattgcatcttttacttctatttttaaaaacttggtgaaaacttgttcttcccactatgtggtctcagtgatttgtatgcaggtggatttgaggattaatggggcttgaagaacaagctgatcaatttcagtcagttctaggcagcttttttgttcttgtttcaatggtgactgcagtcaaagttgggttagtaatatgttgctttgagatagtttggttgaatgaatgttcatgacagattcctcatacctatgaaaatttttaggtgaaagatggaggctgtttttccttctctatccccaatctcaatctcaggaaaccacagtaaaaacaaaatccttccactcagaacctgtcgtgttttgccgaggaagttttctgtgaatgtaacttattttctgttttgtgttttagtctctggccctcgattcctcatctacagcacaagaagttttggactagctttaaaatttctttcaaaaattctatgattcatatgggagaggccagaggttgtgatcacaaatgttttcctggctgattccaaatttagaagcaaactgtggctgtttgacttcccagtcaaacttgaacatagcatggatttcacatttaggggtgttttactaccacctccagaacttgcttcagtgaaatatgcatgttcttaaagatagcaggagtactttttttcatgtagttatgaaagacatttttctcttttattttgggggtaatattttaccgtttgttccttgttggctgaaaaatctaatgctaataaaagctactagcttgaatagtccaaaagcaaacccaacagatagcagtaaattcatttttctttttccctttcctatagtactctgccaaaactggggctttttttcttcatcctggctgcctgtgtgtttgaaaatagatggtcagcttaccataaatatattcacatgtagaaacttatatgtgattaattggcttgatttatttttatcagaagaaaacgtatgtatgttccttctctggcttatgaatactatccgtcttatgtttgctttggataagcattttagtcttcctgtatgtcctaaaacgcgcgcgcgcacacacacacacacacacacacacacatacacacacacacacacactttctaatattgtactgaagccagtttgcaagtctggttccacagtacaattgctctgaaggaccaggagaaattgaactgggaaggaaaatttgtattaacttcaataattcccagacaagttagttcaaatcatttttatctagtatttgctatttgtcacaaaggattcaaaacataaatatgttgtatacaaaatctcaaagagatgtctcaatacccagatttgggtcctggctctgctgcttcatagctgtgtgattatggaaaatcacttaacttcagtggtctttacagttgttttttaaactagagcagcagctcttaggattgtttggagggtcaaatgagaagttttatcaacgtgttttgttaaaggtaaagtactatatagatgtcattcatttagacacttactgagctgcctgccatgtactgagtggagtccagacctgtgcataaacaaagcaaagcactggggctatcttaaggatttaggtgttcagagatgtttcactgggagtttggaagaaggcttgaccactgcagtgcagtgttctgggaaagccctctggctgaagagagaatcaaagtggaggtgatttttagggagatctgtaacggcacgaaggaagtaggaaaatccaagatatgttcaagggatggtcaacagaaactttattttagggtgaggtagttgatggggagtatgactaaggtttccaggaaaacatatagggaagttaaaaaaaagatagctcttttgtgggggatggaaagagaggaggggcgtgggtagtgagtttggcaaaaggacttacttgtgaaagagagagttttaacagatagtgctagacatgaggctagagctggaggaaccagaggtagaaatggggagccaagaagagttcccctttcagaggccggccattgctttggtcttggatgtgaaggtcccagaggaaagcagaacagaaggtgaggcagaccatcctttatacttgaagtgctaacgtttccaaaaagcactcctaaacttctggacctggttgaagaagaatgtgagccaaggcatgaggaagccaggagctgtaagacagtgatgcctatgaacagatttttctgaacatttgtgaacaaggataaggttgaactcttgctgataatgtgcttaatggtttgaatcatggattattgggccgattagctggtcagagcccttttgctgatgcggctggctgacttgcttactggccaagagctgggagtagaacacagatgtcccaacatggtgctgcatttggaagtgtttcataggagtgttgaaatcaatgcatttgctttagggataaggcaaagttatgagatcccagttataagtaatatagttgtgaattctactctttttctcaaggtcatagtaccctcaacagagctcaactagcctttctaggcctcagtttcccgtcacataaaatggaggattatcatgaagttttgaagggaagatatatgtgaaaatattttgaaaccttgaagtaccgtatgagtatgatttgtataattccttaataaacctgctgatgtgtcacagcccccacagggcctgcttgtcagccaggtgatcctgatagatacagcccttgagccaaaccctgaggagcatgtgtattaaagcagacggatgaggcataaatgcgtttggtttatcacttggcagagttggatgacaggcttgggtgggggtggcaagaattaaagtgagtcaagattggcagaaatgtaagacgtgtatgtagtggttttcttctttttttaagcctttgtgattatgtcaaacttttgttaatataattgatgatggtttgaggcagtagtttgggggaacaggatctcaggtattagatctggattcaaacttcagtggtgaccatggacaaattaacctctctgaaccttagatttttcatctatgaaatgggagtactaatactacctcttagggtcattgtgaagatagtacaggatttagtgtatattcttttgttgaggatttcttgtaatgattttcattacaaagcttctgacacattaccagttagatgcttaaatatttgtacatgtgcttaatttttttcatatctctaatatttctagagaattattataggtactcacaagaactgcatctggattaagacatattagccctggctaagcagggtgactcacacctataatcccaagatttttggagtctgaggcaagagaatcacttgagcccatgagtttgaaaccagcctgggcaacatagtgagaccccatctgtataaaaaaatttttttaattagcctggtatggtagcatatgccggtagccctagccacacaagagtctgaggtgaggggatcacttgagcccaggagttttgaggctgtcgtgagctatgattgcgccgctgcattccagtctgggcaacagagcgagatactgtcttccaaaaatacaaaaaaatttattaggggtaggtagtggtgcacatctagagttccagctatttggaaggctgaggcaggaggatagcttgagcccaggagttcaaagctataggacactgtgatcgcatctgtgaataaccactgtacgccagcctgggcaacatagtgaggtcccatatctaaaaggaaaaaaaaatgtactacttgtatcactggactcaggatgaaaaaagaggagggtgggagataggtttaggcagagatcatctattcaaaatgcctttctttctttttcttttttttttgagacagggtctcgctctgttgcccaggctggagtgcagtggcactatctcggctcactgcaacctccgcctctggggctcaagcagttctctgcctcagccccccaagtagctgggattacaggtgcccgccaccacacccggctaatttttgtatttttagtagagatggggtttcaccatcttggccaggctggtcttaaactcctgacctcgtgatccacccacctcggccccaaagtgttgggattgcaggcgtgagccaccgcgcccagcccaaaatgcctttcttgaagctatgggagatagagaagaaaataatcagattaataatcagaaaacagtaatcagatacctggtcatctggttataatagcttcaagcatatatggttaaatagtttttgttttgttttaaaacaaaattttaaattttaaacaaaatttaaaaatttaagagacatggtctttactgtgtcacccagactggagtgcaggggtgtaatcatggtttgttgtaaccttgaattcctgggctcaagtgatcctcctgcctcagcctctgcagtagctaggactacaggcacatatccctgtgcctggctagtaaataggttgaaataacttaaagttatttatttttatttatttattttttgagactgagtctcactgttgtcctggctggatggagtacagtggcatgatttcagctcactgcaacctctgcctcctgggttcaagtgatcctcctgcctcagcctccaaagtagctgggactgcagatgtgcaccaccacacctggctaatttttgtatttttagtagagatggggtttcaccatgttggccaggctggtcttgaactcctgacctcaggtgatctgcctgcctcagcctcccaaagtgctgggattacaggcatgagccactgcacctgaccaaattattttttaaagactataatttaataacaatttttcttccccttcattaagctttaatttgttaagagttaatggctctggtttaagttgatagctctgaaatagcaccttgcatatttcacccctttctttaactgagcagctgctgttagctttgcactctactgatctctgtgaagtttcaagaaatattagtgaaatgtgataggtacttcttaggaactttgcttgaaagaatagcggaccagaatgttgatggaattattttttattatgtgcatattgcttttttaaacttttaacaattatttaaaaatagagatgaggtcttactatgctgcctaggctggtctgggattcctgggctcaagtgatccttccgcctgggcctcccaaagtgctaggagtataggtgtgagccaccacacctggcctatgtgcatatttatttaacctactatttcaccacttcctggcctatattaaccctctgttccagttaaagtgatcttctaaccagtcccttcagctccccatatggtctgttgcttctccatctttgttttcatgggtttcttcctgtctgaatgcccttctagcccttgttagtttttcctgttctcttctataattttggactgtgccatgtaatacaacacttctgtcttgtgttcagtttcatttatagaagttttactccctaaatgataaactccttaaaggtagggcccagcttgtactatgagtccttcatcacttttgcctcccacaatgctgaagaaagagtagtcatgaaatgtgtagtctggagtcagacagacatgagattgaaaccttgctctgccacttaccaactttttgactttgagcaaattacttaacgcctctgagcctgctgctttctttataaattaacagtgagtaccttaatatataatactaatataataagtgcttagtaaaggttaaatattattgtttttctttaattcagcagttgtttgtcgagcacctatacacatgaggcacttatcaggtcctgggaactggtgtagaacaccagacctctgccttgatggaactttatggaccaatagagaaaatggcactaaatgatatataattataaatgtagtaattaccaagaagaaaaatagtaaccacaggaataattgacaggtcttttttagtctggggcattaggaaagattcactgagaaattatttaaactgatacttgaatagaattttgccaggaagagtatctcaggcagagagaacaacatgttcataaggcctgggtctagaaagagcttggcatactccaggatctggaagaaggccagtgtggttgtagcagagggaatgggggaaggggttagcgcccacacccaagggagaagcagcaagcctttatggttctctcggctcagtcagctggcaactggagcatatctctctgtcactttcttttctttaaaatgaactaagatataacaattaaatgcaaaaattatgaaatgtagctatttattgagaaatgaattgtttcatggaagtgactttcaaaaactaagcctacccttttataagtgccaaaggttttttgtttgcttgcttttttaatggaaatctttctaatatacttctctgccttcttggtcaacagtatgagcctaggtcactaagaagagaataaattatgctataccaggactgctgtggtttgaatgtgcctccccaaattcatgttttaggaacttaatcgtcaatgtaacagtattaagtggtgggtctttaagaggtattatatcatgagggcaccaactcatgaatggattaaggcctttatcccaggagtgggttaattataatagggagagagtttggtcctatttcttttctttttgttgttgttgttgttgttgagacagagtctcactctattgtccaggctggagtgcagtgttgtgatctcagctcactgcaacctccgcctcccaggttcaagggattctcgtgcctctgcctcccaagtagctgggattacaggtgtacgccaccacacctggctaatttttgcatttttagtagagatggggtttcaccatgttgcccaggctggtttcaaactcctgacctcaagtgatccacctgccttggcctcccaagtgctgcaattacaggcgtgagctaccatgcctggcctggtcctatttcttttctgtgtctcctgtgcttgcttccactttctgccatgggatgatcctctgcagatgcctatgccatactcttggacttcacagcctccagaactgtgagctacataaacgtcttttctttataaattgccctgtcttggatattctcttatagcaactgaaaatggactaagacaatgactcacagtattttaatgtggtatgacatttaatacccacaggagctattgagatatgtggggctatcttctcccatttcagatgtcctggattataaacttaatgccattgtctgcctgtttgcttctttgctgcagaaatgatattccctttgtaattcctcttgtaatatcctcttcctcttgctgttgtccctgtctgtctctacatatatcaccctcaaatttgatttgcttcctctctatgctgagaaagcagatttctaatattagacaattctaaccggtttaataaattgaattagagtgatggtctgcaaatttctcttagaagtcaaatgcacagatttttaatgcatgatagtgacatcagaggtctccttgcttcttttcggtgagtatccgggttgctgaggcatctgaatcatggaggttgctgttgcccctctcctttgaacactggcttattgtggagcagggagctgatctcgaaaacaacaaaaacccagatacatagcagttgaaaagcaaactgagtggctaaattattgatgagtcatagaattttaaagcttagagagactggaaagattgtcattctagcccccgcacttcggtaatgatgtcaggttgtgctgcccaatttcacgcagcgttttgtagagctggaaataaaatttgggttgactgattattagcctggtactctcctctgtatcatgctccctcagaaaactctgaataattaaatagatttggaaacttgtacattattttgtagttaattttttcatttccaagaatagacttcatgagaactgggacatttgtgatcttatttctggctcatgttttatgattctagcagtgcctaatttaacagaactatattctgttaggattttcttcctgcctgcaaaagcatcactcaggttgtgttctaggcctcactggagtttggaagcctaatcctagatgggcagtctgtatgggatcaccgatgccttgcctttgtaattctcagttttacccctgaggaaatggattcccaacagataagatgatttgtgcttaccttcccaaccagttagttactgagctaggcttaacgttcttttgtagttttattttcattgtgacaggctgacttctgtagattaaaagtctagtatgtttagcctttatttttagatttctgtacataagatgcctgttgataggttataatcttcagtagctcagagcatgtattatcacagcacatggtaggcatgcagacatatttgttgaaagagggcaggtatgtgaagaatgccatcattgaattcacttccttctttgcttgtcaaaaaggctgctcctgatggggtcaggccatcagcaagtggcttttaagctgggccttgccttgggaaaactgacccacatgcaatagtgcaggtgttagttttttcagtgttttatttttgatcatgtgtgtaactaaacatgtaagtttctcatgactggatttttttccaacctacagtgactcttgggaccaactttaaattttccaagcttatagtaacagtaatttaatttggtttctagtttaattagatgggaagatggttgtttccatgaagaatcttgtgaacagaacctagggggtattcagacgatcgtagaaatgaacattgtgctaattcatgaactctaaagtatgggggccccctgctttaagatggtaggcaataaatgcttggatgagtcttatatcatccgttttcaaggtggatgcattttagagaaggtagtacaagctgtcccctgctttctaacataatgaattcctgaaaacctatttgacagtcagatggcaactataaatgccagccagttacttctgcttcccagagctcccctaccgagggcagtagatggctgttggtaccatctgggacaccagaaatcactgttcaataggtaatggacaaaaggctctgaatgtgactctgaaaaggaagtttagtgttatcagtcattaatgatattaggaggtctaccttacatcaagaccacctatgttcctgtgcctcagtgtagcaggggctctttgacctatcctgtaaccttctggtatatctgttaactagtgtaatataaataagaataaactaacatgcgtccaggcgtggtggttcacacctgtaatcccagcactttgggaggctgaggcagacagatcacttgaggtcaggagtttgaaaccatcctggccaacatggtaaaaccccatctctactaaaaatacaaaaattagccaggcatggtggcatgcacctgtaatcttagctactgggaaggctgaggcatgagacttgcttgaacccagaaggtggaggttgcagtgagctgagatcatgccattgcactccagcctgggcaacagagtgagactttgtcccaaaaaagaataaatagataaactgatatggtctatgtgtactttttatatcaccctttcctttatactttttctttccttccttttttttttaagtaaaaatagcttacttatatagttttatttttaaaatgatttctgccaattggaggtgggatgagagtaatttatgcactctagaaaaactataaagaagagtgcacccctatcaattctataataccacttaaagacagttgctattgatattttaaagggtatccttccagataccttttttttttcttttttaagggacaatcttgctctgtcatccagactagagtgcagtggcacaatcatggctgactgtagccttgacctcctgggctcaaacagtgtatctgggacgacaggtgcttgccaccatgcctggctagtttttattgttgttgttattgttggttttttggaggcaaagtctcactatgtttcccaggctggtctcaaactcctgagcttaagcaattgttctgcctcatcctcccaaagtactaggattataggtgtgagccactgtgcccggcccctgataacattgtaaccataaatatgcacatatgatgtcacatgctgttttgtagcctgctttttactaatatgctctgagtatctttcaatgtcagtaaatacctgaaacattttagttttgggttttttaagatttttttaaacgtttcttctttttttccttgttcttgaaacatttttaatagcacctgatatattgttgtatggcataacataatttacttaacaaatctccagttcttcatttataatatacaggtatagtaatatctcatggggttatgtggattaaataataatgtgtgtaagatacctaacacagaagatgacatgtggggaatgttcaatagttgttagctgcagtaatcaccccaacatatgacatcttttataaaggtatgtgattatccccttatgacaaattcctaagaataggatttgctcaggcacatgatacggacgttctcagtttttatttgcatcaccaaatagctctccagaaaagttgcagcaatttatgtactcccacccaccgtagaagaaggtgctctggtccctatgccctcactggatacattttgtcaagtcttttaatctttgccagtctgaaaggcaaaaatgtcatatttttctttttctttcttggctcactacaacctctacttcctgggctcaaaccaccctcccatctcagcctcctgagtagctgggactacaggtgcacaccaccatacccggctaatttttttttttttttttttttttttttttgtggcgacagggttttgccatgttgcccaggctggtctcaaactcccaggctcaagtgatcctcccgccttggcctcccaaagcctgggattacaggtgtgagccactgcaccggcctgttctttttgttttagagattaaatagcttttcaaatgtttattggccatttctttgtccttctatgaattgggtgctcatgttctttccctattttccgaaaacctgtttgaggttttggcctgtttcctactgatttgaaaaccctttctatattaatattttgtcatgttacaaataattttatattaattgaaaccctttctatattaatactttgtcatgttacaaatatttttttccagtttttttcagctttctctttggtgttttgcaatttaaactttttacatagtcagagtaaatccttaagtatgagttctgatttttgtaccatttttgtaaaggtcttcccattccaaggttataaaaatattcgcttatacttctagtatcattttttgacacagggtcccactctgtcacccaggctggagtacagtggtgccatcacagatcactacagcctcgaactcccaggctcaagcaatcctcccacctcaacctcctgagtcgctgggaccacaaactcatgctatgccaccacacctggctattgtttttttgtttgttttcttttttggtagagacagggtctcactatgttgcccaggctggtctcaaactcctgggctcaagcagtccacccacctgggcctcccaaaatgctaggattacaggggtgagccactgtgcctggccatttctagtatttttataatttaatttttttacatttaaatcttcgccctatatggaatttatttaggcattttaagaagggagttaggaattcagctttttattttattttttttccaaatagccagtttttccagtgttcttataccactaacttgaaatgctagctttatcatgtgctaaaagaaacctcatccattcctggatctgttttgctctattgattagtctattcctgcgccagtaactctgtcatgggattaataaatttttccaaaagtggcaataatttcatgctcatctgctcctggtagaatcaagctgacatttcacccagctactcttaaacttcttggcttctgtgcttcctatcctagggtaggattaggtcatctgaatgactgtatttgctctgggctcctcagggtactggcttttggactgttaagcacagaaacagcatatttacatagttttccttaattttgacctgtttctagatcatacgtttagctgactccttagcaatatgaagtgaccagatcgtgttctctttgtgccaaatgaagcttgtccaacccacagcccgtgggccacatgtggcccagggtggctttgaatgcaacccaatacaaattcgtaaactttcttaaaacattatgagatttttttgtatgatttttttttttttagctaattagctattgttagtgttaatgtattttatgtgtggcccaagacaatttttctgccagtgtgccccagggaagccaaaagattggacaccccaacaatctgcctcttaggcatgctacattagagcaacacagctattttttttttttctttttgagacggcgtttcactttgtcacccaggctggagtgcagtggcgcagtctcggctcactgcaacctctgcttcctgggttcaagcaattcttctgcctcagcctcctcagtagctgggcctacaggtgtgtgccaccacacccagctaatttttgtatttttagtagagacggagtttcaccatattggtcaggcgggtcttgaactcctgacctcaggtgatccacccgcctcagcctcccaagatgctgggattacaggtgtggtgtgagccatcatgcctggcctccagttattgtttaccagaaacaaaacatgcttcacttctccccacactctccatttgaattatcatttatacaactaacacataacatctttatatatatatatatatatatatatatatatatatatatatatatatatatatatatatatatcattcactctaccttctgttggctggactctctaacatagtactagatgtttttatatgattcattattttgatattatagaacattcttcggtaatactgtgtcagaggttttgggttttcccttcctatagggattattccgcctttcagttatttctggcaacaaagctttatcctctgctcatatttcatttattcttcttactttgccttgattacagcaattgagccataacttattggctcattattttcaggcctatgtatcatcagacacctttcctgtctaagtgatccagtggcctctctcagctttctttgggtctttcaagcaggagtactatttctaaagtgtcttcttaaatatatttccttttcactatttttattttccttctatttcaaccttaactttctttattctgaccttgagattcttcttattattattcttttaaatatagaccgggtctctctctgttttccagactagagtgcagtggcatgatcatagcttactgcagcttcgaattcctgggctcaagtgatcctcctacctcagcttcctgagtagctagaactacaggtgtgtaccaccatgcctggctgatttttaaactttttgtagcgatgggggcggggtctcactatattgcccaggctggtcttgaactcgtggactcaagtgacctcccacctcagcctcccaatgtgctgggattacaggcatgaaccaccatactcagcctgaaattctttttttattttaacaagtcttaatagcatttactgtatactcagcattttactagatggtgatattaatagattttatttgtggtgttctttgaaaattttcaagatacttgcatattatcttcctgtgtgttctacctgcaccgctgccttctctcattgccttgctcttgcttatatctgcctcccaactctgtaaactctgcttaaatctcagtagtcagacctttatttcttcacttgaaatttgaatttctcttctatatagactacccctgctcagtgcctctaaacacttcgacgtgttcagggcatctttaccaatgtttagctacgtgaagttaatggtaactacaatgggaaagagaggagtaaggtcaccatcatgtgtgaacatctaagaacacataccagtgtataacagtgtcttggctggctggaagagcataggaataaacgctattcagccattatttactgagacagcagtaaccgtgttagaaggaagattacatacagatcaataatgttagttcaggacaaacagctatcatgagttaaaaaaaatactaagaaaaagaaataatgttagtagcaggagtttgtgatagacttttacagagatgtttgtggagcatctaataaatataagaccttgtgctaggcctgatgggtaataccaaattatagcgtctgccattaagaatttatagcataatttgagaggaagacaaatatgtaaatagttctacatgtatagtttacatgtacatatagtttacatttatatataaatataaatagttatagtaccaggagtgtctgtgaatgctgtgaggataatgcttaggaagaggagatcactttgagctgtgataattacaaaccagaatggttggttttacagtgaaagatgggacttaggctgtctttcagatctttttcactggtatagtctgtcccctgtggctgtcaaaaagaaacacccacaatagaatgctgaggagcaatggacaagtgcagaaacacagaacttttcctttttgaaagttgcatttttcaacttttagatttaggggtacatgtgcaggtttgttacctgggtatattgtgcgatgctgggatacaaatgatcccatcactcaggtagtgaacatagtacccaaaagttagtttaccagtactcacctgcgtccctcctctaataatcccgtttctgttgttgccatctttatgtccatgagtacccagtgtttacctcccaatataagtgagagcatgtggtatttggttttctgtttctgtgttaatttactgagggtaatggcctccatgtgcatccatgttgctgcaagggacatgatatcattcattttatggctccatagtattacatggtgtatatagaccatattttctttatccaatccactgctgatgggcacctaggttgattccatgtctttgctattgtaaatagagctataattaacatagtgcatgtgtctttttggtagaatgatttgttttcttttggatatatacccagtaacaggattgctgggtcagatcatagttctcttttaagttcctttttttttttttttttttgagatggagtctccctctgttgcccaggctggaatgcggtggcgcgatcacggctcactgcaagctccgcctcccgggttcacgccattctcctgcctcagcctctccgagtagctgggactacaggcgcccgccgccacgcccggctaattttttgtatttttagtagagacggggtttcaccgtagtctcgatctcctgacctcgtgatccgcccgcctcggcctcccaaagtgctgggattacaagcgtgagccaccacgcccggccctttaagttctttgagaaatctccaaatcgcttttcacaggggctgaacattccctccaacagtgtataaacactcccttttctccacagcctcaccagtatctgtttttgtgtgtgtgtgtgtgatgttttagtaatagtcattctttctagtatgagatggtatctcattgtgaaacctgcatttttgaaaaatagtttcacttgctatagaattacagcttgactgtttttttgccagtactttaaatatgttactccactgtctggcttgcattgtaggaagagtcggctatcattctttgtttctctgtatgtaatgtcatcttttttgtggatgcttttaagatttttctctattgctgattttagattatttgactatgatatgatgtgctttggtgtatttttcatcgtgtttcttgagcttagggtttctgagcttcttggatctctgggtttatccttttcatcacatttggggaaattttggctattatatcttttttttttttttttgaaacggagtctcactgtcgcccaggctggagtgcagtggcgcgatctcggctcactgcaggcaccgccccctggggttcacgccattctcctgcctcagcctcccgcgtagctggtactacaggcgcccgccacctcgcccggcgaattttttgtatttttagtagagacggggatggtcttgatctcctgacctcgtgatctgcccgccttggcctcccaaagtgctgggattacaggcgtgagccaccgtggctattatatcttaacttttttttctgtctccagcccagcctcctctagatgccctgattgcatatataatcagctgcttgaagttgttctacttcaaaaagaatgaacagagccatcgctgagctgtagaacaactttaagaacatttgtagaaattgtagaacatcttctgaattgatagctctgttcattttttgtctttttttatctctgtgtttcactttggatagtttctgttggtatggcttcaggttcacactcacctcttctacagtgtctaatctcctgttcagcctatccagtgtttgtttttttgtttgtttgtttttgagacggagtctcactctgtcactcaggtggagttcagtcgtgcaatcttggctcaatacagcctccacctcccaggttcaagcgattctcctgcctcagccttctgagtagctgggactacaggtgtatgtcaccacgcctggctagtttttgtatttttagtagagacaagttttcaccatgttggtgaggctggtctcaaacttctgaactcaggtgatctgcccgcctcagtctcccaaagtgctgggattacaggcatgagccacagcaccctatccagtgtacttttcatttcagtcattgtaatactcatttctacaagtctaatttaagtctttttcttttctttctttctttttttttttgagacggtgttttcttgtcacccaagctgtagtgcaatggtccaatctctgctcactgcaacctctgcctcctggattcaagtgattctcctccctcagcctcttgagtagctgggattacaggtgcccaccaccacacttggctaatttttagtattttagtaattttcgtatttttagtggagacgggattttaccatgttggccaggctggtctcgaactcttgacctcaagtgatccgcccaccttggcctcccaaaatgctgggattataggcatgagccactgggtctttaaaaaattttaagtctttaaaaaaattttttttctttaacattctcaatccattctctaatttcttgtggagtacagttacaactgttgcaatgtccttatctactaattatcttgttatttctgagttagtttgatttttctcttcattatgggtcctgatttcttctttgcgtgcctggttatttttaaaaatttattttaagagacagggtcttgctctgttgctcaggctggagtgcagtggtatgatcatagctcactgtagcctcaaactcctgggctcaagagatcctcctgcctcagcctcccaagtagcgagggctacaagcatgtgccaccatgcctagctgatttttttaaattgtttttgtggaaatgggggtcttgctacattgcccaggctggctttgaactcctggcctcaagtgatcctcccacctcggcctcccaaagggctgggattatagacataagctactgcacctacccacctgatgatttttcagttggatattgtgagtcttagcttgttgggtgctagatatttttgtattcttttaaatattcttgaactttgttctggaacactggttacttggaaacagtttgatccttttgggtcttgcttttaagcatttttaggcgggaccggcacagcgttaagtctggggttatttttctttaccacagaggtaaatgtttctgagtactctacccaatttccttgaaataacgaggtttcctattatggttgctggtaacaggaattattcctgttcctgtgtgaggtctgaatattgttctctgccgtcctttcaggtaattatttccctggcctcaggtagtttcctcacatgcgtgtgctgatgggtgttcagctgaagacttggtggggtggctggggaggccccctgctcagatctccagaattttttttctgtgtaagtctttcttctctaatcttctgccctgtgaacctctttggcctccccagacgatatatcagcttcctctcttcagtttaggaagaactctggactctgcctgggtgcccctcccctgcactgtgcctgaaattctctccaggcagtaagccagttagctgtagggtcctccttgttcagttccttgtctttcaggaatcgtcgtcctttgtgcctggtgtccaatatcttggaaacagtcatttcatatattctgtcgttttgggtttttttttcttttcttttcttttttgagatggagtcttgccctgtcgcacaggctagagtgcagtggcgcaatcttgcctcactgcagcctccgcctctcgggttcaagcaattctctgcctcagcctcccgagtagctaccgcgcctggctaattttttgtatcttttttttttttttttgagacggagcctcgctctgtcgcccaggctggagtgcagtggcgcgctctcggctcactgcaagctccgcctcccaggttcacgccattctcctgcctcagcctccggagtagctgggactacaggtgcccgccaccacgcccggctaattttttgtatctttagtagagacggggtttcagtcgaactcctgacctcgtgatccgcctgtctcggcctcccaaagtactggggttacaggtgtgagccaccgcgcccggcccattctgtgtttttgttttgttttgttttgttttttattagctgtttccggtagaaaggtaaatgtgatccctgtcactccatcttgaccagaagcagaagtccacccagaaatgaacttttacatttcatactcatgtctgtcatggggaagttaagtgggcctggccttatccttttacttgggcgtcatctttttcaaaacttcataagtagttcttaaagaattcaatatttattgcgtgcccactgtgcactatgctgtgtgttagctgctgaaatgtatgggcttgattcctttaccagatggacctcaggtctagtggggaggacaggcaaggttcttgtggctacaattaaaagtgctgtaatacatattttgaaagatctaagatgccactgcagtgtagaatgacatgtacgttgtcttaaatattagaaatgattctggattaggattgatggctttgtgctgacttgtgaattctgtctcctaaccatgaccaggttgtctgccctttcctggacatccgtatttttttctcccattgaagtggcaggattgaatctgtactcttgggtccctagctctcctggatttatctcctttccatatgaccttttctttctgcttaacctttgggcctgctgtaacatctcatttcatttttcttttttaactttcaccagtataagaatgttagagactgaaatattaaaaaattaattacatttgctataggtagatgagtttttcagtgagtttgcccattttcaaaagcaaatcttcattggttatttggagtgggtgggagggacagcttaagggcagtagaaaaccacagttttgctagtaaatctggcctacatttccaaaggaactctttggatgatgagtgaggtccattaccatttcagttgtttcagtgaagctgagagttgccggtgacacgtgataatcctcctcattctgtagttgagcaataggaaaatgtgggtaatgccagtctgcattgactgggtcggctgtgtagagcattgcctgaaaagtaaaagttgcagtcctgattggctcaagactgatatcttacagcctgatgaagacaaccacgaattccagtgccataactgatggactttttcttttctcttagttatgacaaggaccttccttggaaagataacctgtgtttagatgactgtgatcttagcccttgtgatctgcctggtgcttgtggtagctctggatgttcctgtttttcctgaaggacctcattgatgtcatttcagacactatcctcactcaagagaatgtgaggttttgctatgtgtgaaatatcatataagacacactgctatgtcagtttttaagcctctggtttcagaagctctggcagccctactttttctcagatgcctggcaagcctgcatgtctgaatagcccttctttcctgcagaggctgtgagaagctctgccgagcatttgagagttaaccattatttacggagcaagacctgcgtgaggcactgattgtactaacttacaaaaattgtgtaagagattcttttgacatcaaaggtacttacggtccagaggggaaatcacatacgtgaattcggaagcccattcaagactgcaagagaaaattcgaagataaacaaaacagttcatgttttgagttctgtttctttagatttctatgtgggattggtggcttgagtgaccaagtagctggcatcccaaccatttatagacatgttctattttgaggttgagtttgtggtttggggaggatgtgtaatcattttcaacaaatgcttactgagtgtgcaaagtattcctgtggaaggcacagggagtaccaagaagtataaagttacattccccagcttccaaggagcttctcatatctgggagggcaaggcatgtttaagattttaaaatcttagtaagattataagtcttaagaaaaaagcataatcatccacatttattagaaatatatactgaactattaaactgtctaagaaatggggcttgggtggtgagcatgtggcctggaaaagagaaggatcttcactctttgctatatagactctatatagtatgtaatttctatatggtttgaaatttttgccaagtgcagttattacctctttggaatttttattttttttatcagttacaaaaattatagagaacagacgattgtattaatacatataatattaaccttgaatatctctatgtggtaggattatgagtgacttatctttattatgcgttcttataattgtctacatcttctttaatgaatatatattattgtataagtagagaaaggaagtatttgaaaaacaaaacccaaaaccacctacaatgattattataactatattttaacatgcttactatattttggatataatataccagctttcccttttttacctccttactctcactgcaactctagtaaggttgttttattattatcattattactattgttattaaataaaaaggtgaaacaaaggcatttgcttcttgcttctgcccgttgtcatttttgagtgagctagactagaagtaaaaggaatccagttacgaacttttcagctatactgtaaaggatgcccagaaaaatttaaattatgttgatttggttacaattacgcctgccagtgtgggctatgatcttttgttttaaagcatcctaaaaatactttgctatacctttatatttagttctgttggagaaagcaattagattgtcccatctcccatctccccatctggtgcagtaaagaaaactcatctgcttggcttactttgtgttagagagaatttaaatttccaagttatttgtgattcacattaagtagttaaatcccatccctcccaaattttgtactgggaatccatacccactttgtaattatgaacttaattactctgaacagtaataaagttcagatatttaaagaagccaaagattgaaggagagtggctgtcatttctaaattaattttttttccccttatagcatgtgtttgatgatctcagaggctcagtatccttgtcctgggttggagatagcactggggtaagtcatattttgagtctgttatttcagcccagcaagaagatgataacagatttggggaagggttaagcacctaatatcatttcttcctgaaactgtctttagcctcaatcacagaaaacacatgtttgcaagacagactttcaggaagttgtagttgtgttggttttgctttagaattgggaacatcttttttgcgggggggtggggacggagtttcgctcttgttgcccaagttggagtgcgatggcatgatctcagctcactgcaacctccacctcccgggttcaagcgattctcctgcctcagcctcccaaggagctgggattacaggcacccaccaccatgcacagctgattttttgtatttttagtgaagatgaggtttcaccatgttggccaggctggtcttgaacgcctgacctcaggtgatccacgcgcctcagcctcccaaaagtgctgggattacaggcatgagccactgcgcctggccgggaacatcttctgaccaccttcttccacggtgggaagatggacaccttttcagtccttggtggctgcttagcatcaagatgagcaagaagaaagatgaaagtgctctgtactgagaagggaagcgcacaatgcttggggaaacccctatatagaaacagaaaatgtccattaaaaacatcctggcacttttcctccaaattgtattgttcctgtgtcctttcctacaggtgtgttgagggactgtggtgcccatgggctcatggttgttcatctgttggtccctgcccctgccacaaaggaggtactgccaggattgggggaatggaaggaggatcagtcagctccttatccctcctgatatacagctgggctcccactaggaggacacgctgaggatacattggcccctggtcttaaatcaggacttgttgcctggctgtcttattatcctgcttgaacaagagcaggggcaacatctcctgccccctggtattccataatgcatgtgtcatagcattgaggcacatgatagttatattagaagcctaaagaaataaaatcttggtgctgaaaagcaaattgtaaatttcttattttgattaatttttaaactttcagtagaatttctttaagcatagttcagttttttctaaaatttgagttgtaccctaatttccatgatttacccatttatcttagatagttggtatctccaagaatttttttttttttttttttttttgagacagagtcttgctctgtcacccaggctggagagcagtggtgcaatcttggctcacagcaacgtccacctcccaggttcaagcaattctcatgcctcagcctcccaagaagctgggactacaggcaagcaccaccacgcccggctaatttttgtattttaagtagagatggggtttcaccatgttagccaggctagtctcgaacttctgacctcaagtgatccgtccgcctcagcctcccaaagtgctgtgattacaggcgtgagccaccgtgcccagccatctccaagatttctcatatggctttctgagactccctagactcttatagtaatccatccagaatcatttcctcatgcatagtttttggtagataaggtactgaaaaaatgaatttcatgaagaaccaacggcaaggagaagaaaaagacagacatacatgacacagtgatctctgtgagaacacccacgctattctagactctgtaaggagaaaagggaagagggttcttgcctgagagtatcttacaataatttggatacaaaatagttaaagccttcttttattttaatgtgtatttgagtagtcagcctcactgcttcaatggaggcaatggggatcatcccaacaaaatgagctgaggactttctcatcattctcagccctagtaactagatccttctagactttggggagtactacaaagtagggatatcaaaagtcttttaggtgttggcttaatctcgaacacatatcagcatgtgattggaatatcttttttatttacggaatgcttttttctctctccaggtcattctagtcttgactaccttccatgtaccactggtaattatgacttttggacagtccaagctatatcgaaggtgagatcaataacacgcgttggagcatttcactaagtaacattgagaatatagattctcccttttctagacatgatggcacgtaacagttgatgtaaaagatcttctagccatcctaagttttcaccagactgtcacactcagcacatgtttttgttgttgttgttgttgttgttttttgagacagggtctcactcttgtcacccaggctggagtgcagtggcgtcatcttggctcactgcagcctccacctcccaggctcaagccatcctcccacttcagcctcccaggtagctgggactgcaggcacacgctaccatgcccagctaattttttgtattttttgtagagacagggtttccccatgttgcctaggcttgtattgagctcctgggttcgaatgatccacctgcctccaaaagtgctgggattataggcgggagccactgcacccggcccttagcacactttttaacgtctactctttttttttttttgagcagggtctagctctgtcactcaggctggagagcagtggcgcaaacacagctcattgcaacctctccctcccaggctcaagccatccaccatcttcccacctcagcctcccaagtggctggcattacaggcacgtacctccatgcctgggtaatttttgtattttggatagacagggtttcaccatgttgcccagtctggtatcgagcgcctgggctcaagctgtcttcccacctcagcctcccaaagtgctaggattacaggtgtgagccaccatgcctggccttagcatctaattagcaactgaaagaagtatgaaaagggacctggttccaaggaggttggtttgacctgactcaggggcagaacaataaaggaagatgaagaaagagtgtatagtgctgtgtgtttgtgttcatttatatggatatttatttgtgggaaggtagtatggggtttaaaaagtctttctatttagtgtgagtacaaaatggttttttatattcctgaaatgaaaatcttcattgtactttcccagcaaatatttatacagcattttgaggcctaagcagattatttttaggtaagaaggaggctctcaggtcggacctaccaaagtgctttgtagtgttcacactggcgatgatggtggtaacatgtgttctggaaccacaacctatactgtgacattggtgaagttttagagttgtcttctcaaaggtatgacttggttaaaaagtaggttccgttccttagcagtaagattcaagaccaactaactcctagagattgctgaatgattgtgtcttctcaatcatttatgtctgtctcattttcccctagagacctaaagaagtaatgacataccagcagatgatatagaaactaccctagggtcagggtatctcccaggagtaatggtactttctcaaggttctttatttgactgctgttcttggcccaagtgggagtgaacccatgttcaagctctgaaatggattagcctaacctctcttccccgtgaggtatgaactttgactacatctcctgggcatggagccagaggtctctgcccttcaatacattgatgttggtcccagagaggcagcaaatacctttaggccaacaaggaattgccatctaactacagtgtaaattccatgaaggcaaggttcttgactaactggggtccccaggatctagccaaatgcctaacatatagtaggcaggcagcaaataataattgaataaatgaatgaatgtcttggcttctttataatttttagatggttaattaaatttttttaaatataggattgactatattttagaatcttggtgctagaaagcgctatagtaggtgactttgtttattcaatcagaacagtagttgaaccattttagatgaaaaacatcctatttggggagagaaaaatgcattttaaaatgtgtaactttgcttcagtaactctttcctgtggttaagaatgtttgctattataaattccctcctcacaaaattagctgggcatggtgtcacacacctgtggtcccagctacttgggagactgaggtaggagcatctcttgggtccgggaggttgaggctgcagtagctatgatcacatcactgcactccaggctgggtgacagagcaataccctttctcaaaaacataaaatatctcctcatgatagtttttcttaccaacctgtgtttcattataaaagcagtataatcatattataggacatttgaaaattttaaaggaaagaaaaaagttcaccatacagccacgtgtaatcttaatttgtttgcagtttattatcttgaccttcccctttgatgttgtatcatataattgtagagtattgggaggcaatgtggaatagcaaaatgttgccaagctttgttggcaagtaggctcaggattgaatcacagcactgtcattgcaaacagaatgaacttgggaaagtcatttaacctcctaagtcttttttgtcatctgtaaaaggacaataataatgcccacctcagagccgttgtgaggtatgctctgcagtgcctggcatggagtaatagcttggtgctgttagttgctgccatgccaatgatcagggatggcatgtagccagcaggtactggcattgctgtaccagttgttaaaatgaatttgaagccttcaggcagattgcctgcccccagatgcctccacgtcttctacaaatgcacactgcaccccctagcccctgccaacatgcaaatgttactaacacctggcattatgacctacatccatgctgatgggtcggtactcataccttcactctgctggtaacccctcatttttttagattgacataatttacttaatcattgttccattggatagttaggttgtagctgtttttttcgctgtaactaatactactgtgatcagtatcatacatatttgtgtcataggggatgtttatttctagttcactcttcagtagcaaaggaaaacctccactctgatggatgtcaacgtgttttaaaggctgtttgttaaatgcctctttctctaggttgagttatcttctatacctaatgcctcaacaacttaagacaatcaaaaccaaaacaagaaccaatactgccatgtcactgtttaaataccaccaccatcagactcagccttttttcagcctgtttttgtctagtcttgtgaaaaaccccgctttcctgacatactttactagagtgcaattctatttgtttctgatatagtataatgttaccaaggtacttttttttcatgttacccagcagttttttgtttctttttttgtttgtttaaccacattgactttgaatattaattatctcttccctgccatgaagtggaaactgactctgtacccacttccagagtttaggcggaaactgtaaatatgccttgttgcctttccaactgggctggcctggcttaaagtgactggccaaccaagtcaaccatgtcaaacatgccaaccaagaggcaactgttgtttcagaaatgagaaagggaaagaagcagtttgatcaaaatgggtcctgagtatgctgaagttgcccaattaccgataaaagcccattgaacccactctgaagatttttctgtttttcacagcattggtgattatattattcgttagaccatcttcgcatactaaatcttatatggttaaaggttcatttggatttgaaataaaaaacagcaaatcacatgccacctgctctgtgtattacagcatgaagtctcacaagaatagaaggaagttgtctctgttgatatacagaattagagggagtatacttctgtctttaggccatgaaaaagaattattgaaggatttgggagattatatgtatgtgtatgtatgtatatgtgtgtatacatatgtttctgtatacatatgtttgtgtgtatgtacatgcatacacacacatatgcatattttccatttaagggaatcatatttgtatagtataaattttgatttccttataaggaatattagttttataatggattatagagtatgtttgtctctcacttgcacatatgaagttaaagaaataaactagctacaattaagatgataatttgatttttggcaactaaaagatattttccatctttttgttttatgatgtcttgagtccaacttttcattgtataatgtacttagtgaagcaaatgtaggcattattctacaccaatgttatatacctaattattaatatcacaaagactgctgggctattttattcctggtgtttctattgagaattgctcaaatttccctattaagaggttttgcattaaaatatattacatagtcaatgtaagtttttcagaatgttacttgagatttttttaaaaggataacttaaaggtatacaatgcaaagtgctgctagtgcagtaaaccagcttcttcaaggcatgttgtgtaacaacctttttaatgatatgctcagttcagttttcactgcagtagattgttaacaatgactgcatatttcagttacaaggttgggataaattccatttacaacatatattttcttgcttttttttttttaaagaaaaagtatattaaaatgtgatgatggcattttacctttaatacaacttcacttctatacacgtaaagttgtcaatcagagtaagatgaagacccacaaatacatattcatgttccttctgattaaaagttctgtgatttttttatttacaatatcttactctctttattttcccacttgtagtttcaaaatttctacagaaaacatgtatcctttcccaataatgataaaacacaacatagttaaataaaaacacaaggtaatatcagacagtgggggaaaaagacactgaaatattgggaattaagtttattatgaattcaaatcttgccaatattacccagcagtagtttcaggtccatgattattttgatacattttaataaactacattctccctattgatcttttacctggaaatctcagtgcagttggttgtcattattagtgatagttatgttctgtaaagttgtcatgaatattgaattagttaatagtgaaccattgctcctagggggacatatagggttaggtttctgcaaacttctggtcacagcactcattttcatcaaatgatcaatacataactttgttttatgtgtgtttctgtttaaagataccttatttaatgtgtattattgattcattaacactgaattcatggccaatagcactagaactcttgcccaaacaaggcttgtctcatatactcgtatttcctccatgaggcacatcaaagtcttcttgtgtttaggaacactaaacagcttgttgacactgctcttggaggctatttaaacagaaaaagtaccccccaaaaaaagcataaacttgcaaaaacctaagtagaaaaaaactaagtagaccgtaaaaaggacacttatttatagtattagaactgaacacaaaggcaaagcatcaccttgtttgacctcagctgggaatgggtacactgggtgactcacatttttgctgctctatgaatgtctataaatgactggcaagggaccatgagtactgatttggggattacaaataaatttaagaaggtaaacaaatttacaaatatagaatccatgaataatgaagattgtataagctttctcaattagtaaaaaccacaaaacaataatatttttaagagtttaccatcatccttgttacttttatgttataatttatagtggcttagtcatggcaaaatgaacaaaagaaaatgggtagaagcagaaagagtttttgaggtgcatgcttgaaaaagcctacatagctgttaatgaaacttaaagatgattctggtgaaggctcagaaagaaaagaggagggctgtagagaaaaattccatcttagagaatacctaagtaatccatggtagaatattcgtaaaaatgtggaccacaaaggccattatgatgggtttctggcagaaatgaggaacttgttgttggacagtagagaaaaagcaatccttgttataaagtggcagacaactaggctgaattgtgttcatgttctagtgttttgtggaaagtagaacttgcaagcaatgaaattgcatatttagctaaaacaatatctaagtaaaatgttgaagttgcagcttggttcctcctgactgctcgtagtaaaatgtgggaaaagagaaatgacttaaagacagaattattaagcaagaagaaagcagtactaaaagatttggaaaattctcagtctatctaaattgcacagactgcaataataggatgtgaataattaggatgtggccaagtgaccatttgataaagagattagcataggtgtaaactaagaactcaatcagtcaccccaacaggaaaactgccagtttgaacccaatgagggaaggctttcagatttcaaggaccacagagctattcagttgcgtatgtgtaatcttctttaagacaaggccattcagggatcaccaaggctgcctcctcaatttcaaaagaagggaccatcacccaaagccatgggagaagggccacccagatccttgtgggcctaccccctttctggcaaggctgcagagcaagaacacaatcccagcgtgtctgggaagaggagggaccccctccccaatattggacctgtaggacagagcatagagccaaagagaattgttttcaagctttaatatctaggccaggcgcagtggctcatacttgtaatcccagcactttgggaggctgaggtggaaggattgcttggggccaggagttttgagaccagcctgggcaacatagtgagacccatctctaccaaaaaaaaaaaaaaaaaaaaattgagtgtggtggtacatgactgtagtcctagctacttgggaggctaaggcagaagaattgcttgagctcaggagttctaggctatagtgagctatgatctcatcattgcaccccagcctgggtggcagagcaagaccctctccttatttatttacttttgacacagggtcttacactgttgcccaggctggagtgcagtgtcatgatcacggctcattgtagctttgagctcctgggctcaagtgatcctcctgcctcagcctcccaagtggctgggactacaggctggaaaccaccatgctcaactaaattttgtatactttgtagagatgggttttcactatgttgcctttgggaggccgaggtgggaggatgcccagccttaagacctactctttaaaaataactaaataaatatatctcatgaagtttgcctgtattttagacttatttggagtcacctttcgtcttttccatttcccttttttggaatggaaatgtctatactgtgcctgtcccaccattgtattttggaagtgcataacttgtttggtttcacaggtacacaactgaggagcagtttgcttcaagatgaatcataccttgagtctcacctttgtctgatatagatgatatttagatgacactttcaattttagactttagagttgatgctggaacaagttaagactttaggccagatgcggtaactcatgcctgtaatccctgcactttgggaggcagatgtgggaagattacctgaggtcaggagttcgagaccagcctggccaacatggagaaaccctgtctctactaaaaatacaaaaattagctgggtgtggtggcacatacctgtaatcccagctacttgggaggctgaggcaggagaaccgcttgaacctgggagacagaggttgcagtgagccgagatcatgccattgcactccagcctgggtaacaagagtggaactctgtctcaaaaaaataaaataaaataaaaaggcttttgaggctgctgggatgtaatgaatgtattttgcatgtgagaaggacatgagtttggaggaatggagtggaggggcagggtgggggtgtcaggatgccgtacactgaatgtttatgttccctctgaactcatatgttgaaattttaacccctagtgcgatggtattaggaggtgggaccttcggtaagtgattaggtcataagggcagaaccttcaccagtgggattagtccccttataagagagaccccgagagctcccttgccccttctgtcatgtacggttacagtgacaagagggtaatctgtgaagcaggaagcagaccctcaccagacagcacatctgctggcaccttcatcttggacttctcagcctccagaacagtgagaaatgaatttctgctgtttataagccacccagtttataatattcttttatagcagccctagctaaaataagctctattccaaatatatttgtattttagcattattttcttgagttataaacatgtgcatgtttacaccgtggatacatgcaaaaatgatcatttatttttggtaaccagaaaatggatattagtgttgatgcatagaattgttattaaaaagacttgtttctttttttttttttttttaagacggagtctcactctgtcgcccaggctgcagtgcagtggcacgatctcggctcactgcaacctctgcctcccggggtcaagcaattctctgcctcagcctcctgagtagcagggatttcaggcgcgtgccaccatgcctggctaatttttgtgtttttagtagagacagggtttcaccatcttggccaggctggtcttgaactcctgaccttgtgatccacctgccttggcctcccaaagtgctgggattacaggcatgagccaccacgcccggccaaaaagacttgtttctaaagcagttggtttcacagcattttaactgtttacttatcattcgacggctgttcataacgctattaattatcatatggaatttgaaaattagatttatcaaagttgcttcatcaatttcctcttcgtgacataggctgatagaaaataaagtgctaggatcacgcctctaattctagagctttgggaggccaaggtgggaagattgcttgaggccaggagtttgagaccatacatatcttcatgatatgtatgctgatagaaaataaagctacatttaaataaagcattatgttaaaaaatatacccctgggcccaaagtggccttgctagaaccctacttaagtcccagcaggttgatactagttcattttctccatttatttaactgactaaagtaccagagtgctgtagcaggtgattttttgttttgctcatgctggtttctttttttgaagggcagggatgtgtttgttgtgccaagttcttaaaaagcaagcaggcttgccaacaggtttgtggagattctctggggatgtcatgaaggaggaccaatttgtaaactagtactaatcaggctgcttcaacccctttggaagatttacagagctgttgatgatacatgcactgtagagctgggaattgtttatgcacatctcttggccagctgtcaggaagataagagctgatctactgcaaagccaaagaaagggtaatataatataggactctgttgggtggcgcccaattgtatttcatttatctaggaattttctgtgtgtgtgtatgtacttaaggaaggatagagtcattcagacaacttagacatatgtagtttgtttcagcatattatcagggacctctacatacacacatggatacttcccttctgtctccaactttctcttagctgatctcttctgccaggtcataagcccccagcaagacagaggaagaccccagtttgtaagggaaaaaatcctcacagataattaaaagttatctccagtttagggctaaaggttcagagaattggactatagtttcaattttgcccttaattaactagttgacttttgataaattgccttcccttgcaactcctcatctgtgaaatgagggagttggacaagatgttcacttcagccccttacactgctcatggttctgcaattctttatttgttgaggccgctaccaaaacctgtaacatcatttacatcatgtacattcaagcttcgcttaatgatggggatacattctgcgaagtgcatcattaggtgatttcattattgtgcaaacatcacagagtgtacttactcacacctggacagtatagcctgctacacaccacagctatatggtataacttcttgttcctaggcaacaaacctgtagagcatgttactgtactgaatactgtaggcagctgtaacacagtactaagtatctgtatatttaaacatagaaaaggcatggtaacaatatggtttcataatctggactcaccatcatataaacagtccattggtaaccaaaacatcttcatgcaacacataactatattctatcattgaagaccactgcagattacatttatctgtgtgcagaaatttgcagtgcacagcagtcttcattgatggatacttaaatttaatgaacattaagatttagcattttgtattgtgtaagagagctatacatagtattaaacctgtcagggttagacagctatggaatttttgcttttattttccagaatctaaacatggattcgaatacttctcaaagatattgttggaatgaacttttggaaaaagcttggaaactctgagagttgagattaagtttgtttgtttttcctagctgtatttcatgtactgagtgccttacagacttctgcctagggatggtaacttctatggggaaaaacttcaagaacttgctacagcttcatttacagctttttcataactactgtggattaatagattagttagaagcctttgggagcactttgcatagatctcaccttaaactaacctgaataataagggaatagttgattctgtaactaagtacagaggaatgacaggcttcagggtcgattcaattcagtgtcataggctctgcttctcggtattctgaaattaccttctctgtgctcccattgtgacagaatgacgatagcagtctttactgtgtgttcacatactgcatcattggggtttctcaaccttagtactcttgacattggggccaggtaattctttattattgtgggactgtcctgtgccttgtaggatgtttggcaacatcgctggccactacccacttaatgccagtagcacctcagcccttcattgtgataatcaaaaatgtctgtaggaccgggcacagtggctcatgcctgtaatcccagcactttgggaggccgaggcgggcggatcactttgagcttgggagtcaagaccagcctgagcaatgtggtgaaaccccgtctctacttaaaatacaaaaattagccaggcgtggtgacacggacctgtaattccagctactcgggaggctgaggctggagaatcacttgagcctgggaattgcaccactgagctgagattgcatcactgcactccagcctgggtaacagagtgtgatcttgtctcaaaggaaaaaaaaaaagtctgcaaacattaccaaatgtttcttgggggacaaaagcaccctgattgagaactgctggtctgaaggaatatagaccattttatcaggtagctatcataagtgggaggcagtgtgcttcctagaagaccccaaatttcccacccaccttttatctcccattggctcatcctgagtcactccccgattcctgaacccccatcactggtgagtgggtttgtgggggggggggcgggtggaatgttcttcggactcaccagggcacctctggagctaggaatgtggctattcagggtgagccgagtctctcagcatgattggtggaaagggagaatgaaagctgagtaggaactcagtaatatccatgaccactgcaccctaaatttgccctttttttcttcctctcagtgaggattatgggaagaactttaaggatattacagatctcatcaataacacctttattcggactgaatttggcatggctattggtcctgagaactctggaaaggtgagactcattcttgtatacataaagcttgtgcttgataccacatgcagagctgtcagatattagtattggcatggtccagtgattcacagtaatgtattttagtttagtattgctatatttatattttaaaattaaacacaattttttaagggtctcactctgtctcccaggctggagtgcagtgataccgtcacggctcgctgcagccttgaccttccaggctcaagggatcctcccacctcagtcacttgagtagctgggactacaggctcttgccaccactcctgggtaatttttgtatttttttgtagagggttttgccatgttgctcaggctggtcttgaactcctgggctcaagcaatccacccacctcagcccctgaagtgctgggattacaggcatgagccactgtgcctgccttaaacacaatttaatatcgatcatataagaatggaactctgagcacacaggtttatttgtttttgttccccgcccaaggtgcaccaaagcccttaataattatgcagcagttgcttaacattacaatgtatataacctatgacaacattttagttttttggaaaactaatctcagttagcctaatctaggcttaaacccacagatatgatgtcccagctcagttcagggtgttatttttttataagaataagacccaaggggtagagtggaacaactaatcagtttttataactcttcttatttctagaactaaaatttcttaactaatgtctggtgaaaatggtgtaagaactcctgtgcttgcatagatagtatcataccctgaatcagatctttcctcttcctatcaggtggtgttaacagcagaggtgtctggaggaagtcgtggaggaagaatctttagatcatcagattttgcgaagaattttgtgcaaacagatctcccttttcatcctctcactcagatgatgtatagccctcagaattctgattatcttttagctctcagcactgaagtaagtccagttgaggaacatggtttgccttttgcattttatagtctcaaatgtactgtcttagaaggatcctttaggaaatgcttttaatattggacttctttcaagtctccttatggcatgaagtatgtatttttttgacgttttttcctatttggtaaaggacacgatttccacatgctatctattccaggtaagtgtgatgcctagttgtcactgtaatttttttaagtgactaaatgtatggtattggagtaatgaactagaaaacagtttaatttaatcattcttgtgctacaagggacaagcataaattgtcaaactcctgatgttgtcattgttccttttctatgatcaaacaacatctttctaatttttttcttttttgttatgtcctcagtttataaaacatactgatttctgtctttgaccctcatgtattaaatatccaaagtcttaaatccctgcgctccctaccacgttcctatactttccccagagaccagtgtgtttccctaaccctcccacttctcaaagttctctgaggaacacattcagcttctcctttcctggaggaccagtctcctttactgtggttaagggcccaaagtggctgagttgctagactataataatcagaattatactttacatttttatggccttctctcatcagatagaaacacctgaggagctagttcaagaggcacatccctttgcccaaacccagatataataaatcaatgactactgttgtggggtggctaatatttttagcaggctccccagctgagcctgagcaccaaagtttgagagtcattgctgtactgactgttttggggtccaaaacaagatcatcaaccgtgaacaagtcaggctgagcaaaggatgacataggaggacgaagggcactggaagagaggtctgggttctgggctttctgtggggagatttttgagcagaggtgaggctgaggggcaccaggagggccagagttatgttttttttgcttttgtttttttttttttttttttttttgagacagagttttgctcttcttgcccaggctggagtgcaatggctcaatctggtctcactgcaacctccgcctcccgggtacaagtgcttctcctgcctcagcctccagagtagctgagattacaggcatgtgcacccaggcccagctaatttttttgtatttttagtagagacagggtttcaccatgttggccaggctggtctcgaactcctgacctcaggtgatctgcccgcctcagcctcccaaagtgctgggattacaggtgtgagccactgtgtccggcctcagagttagcctagcagtggaggccagaggaaggaaactagcttgctggggagtaagcttcagatcttagaacaacaattcctggaaattggcctccagccgcttaagctgaaagcactataagggagcaaggagtgaacagacccatagagctcccctttgactactgcatcttccttaccaagtgtctcagggttattaaaacagcactgtcctcaggaagagacagatacagtctgcttctgtacctgaaatttcttccatttaagggtaaatctgaagtacatataattgggatggagaagagggaccaatttgtactaaagtgcaaatcagtggtatcaaattctgccttgtctttataagtagaattaggtaaattagaaatgctaggatgtgagtattaccaggggagaaaaagttagaggagaatggtgggtgagagtacaggagtactgaacataaattctgcctaaataagttacatttaatttcagctagactaggacatctctgtcccatctccactgaggcggagctcagtaagctcacattgccggtgtaatgttgtcagtcagaattttgttaagtctctgctacatttcaaactattggccaatccctccttttgagaactctaattgtttggcttctgtgacattattccttttagttccttccttaacctcctttcagcctacccttaaatgcctactcctcatttcactgagcattctcttccactcttaccatttagctaccaccacctataatgtataagattttcaaatccttatctctagccctggctttcccctaatgcatcatattcctgtcttgctgccccctgaatatctctagctggatgttccacaagcaccttaaatcaatttaaaactgagcttcctccctactcttctctcagacctgatcttcttcccttaatccttatactagttaatgatatcacaatgcatcctataccccaaccaggaaactagagtgaccaacttacatgcatgtgcatgcacgtgcgtacacacacacacacacacacacacacacacacacacacacccctacctctcatcctgccctgcaggagaagtcatcccgttctactatgctagcttctaaattcttgcagagcctgttcctcatggccagtgttcaagacctcattagctcttaccgaactaatgtggcagtcattccctgcttttccttgcctctagtctggaccctctgaaatccatctttcccattcacgtccaagtactccacctaagaaagtgaaacggactgtgtcatcctcctgcttagcacacattcctcctgagagcacctcatatgcaataaaccctggagtccttaccatggcacagaaggaaggctctctgtgagctggcctcacctttctgcctagcgttagttcctcatttgcttaccttggaatttgcactcaggtaatgcctgatggcttttagctccctgcacacactgcagggtttatgacctccttgcgtttgctaatactagtctctctttgtgcagcaacacctcatctccctgtttgccttcttttggtcccactgctcatcatttaaaactcagtgcacctgtcttcttcaggacaccatctctgatgcaatgttcctcctctcctcacttctatactagaccatattttataactctgcatagtgttgaactttcttcttatctatgcgccttgttaaactgtgagccccttctctcagcagggagtgtgtcttattcatctttgtatccccagtgcaacatctggtacagtgcctggcacataataggcacttcatacgcatttgttattctgaacttccaagaccccaaaagggcccagataaactacgtgtgtgtctgtggagctcgggcagctctgatggagcttcagtatgtgtttttcctgtcctgaatgattggatatgaaatattaaaggtcatacaagcaactacacaggataaagctagaattttgataagtcatttataatctttcagaatggcctgtgggtgtccaagaattttgggggaaaatgggaagaaatccacaaagcagtatgtttggccaaatggtgagtaacagaagactccatctgtgcagagccctaaacccttcctgatagcgctgccaaaaatcacatgaataaatattgagcacttactgtgtgccaagtactcttcagggtgccataagagatattggagaggggtgtgacacaggccttgacctggaggagcttctcattctgctggaaaagaagacttagtctcacttaaatgttagaacataaagtataaaacaaggcaggtgactgagttgctgctgcctgcccagggttcattctccatgaatggtgacatcaattaaaatacaataaaatatagctttaatttagggatttaaatacttctccatgcccagatttaccctcttgtctagagttccatttcagtcatgtagaacatggcaaggaatcaggaagaaagctgaaagagcttccagactcctgaagtcacattgcgcaccacaactcattcacttttctcggagagattccctgcccctccgggtgcctgcttaaagaggatggagtggagggagctcttacttgggagtctcgtcacctttgttctgtcctcactctgctgctagtcaggggctcaacctttctgggctttagttttttccttttgtgaaaggaggcttatttggagaatgtggggatggctaagcaagaactgaattaacaggcttcatgcctccaaaatctgattaggctcagaatgttgtttagagcctctttctcctttccctttgactaatttcagctaatttgtgaatcccagtttatagcagattggttacagaggtaggatatgtaactgctgcaataagcacgtaacagcattagaattacagcctgagagctggaaggaggtggaattactctaaaagagcagacaaaaacttagagaataaacagtattaaattatttagtgctagatcaagaaagtacacctgtgttagtaacttctaatgggaacactgtgttatagtttgatagaattgatgttcatgatgatgacccacaatcccaagagtttcctgtactctaggaatcaggaaagtgtagaatacagatttgaaatatgtctaccaagattgattaaaatattaaatatattgcttaaataaagtaggccttttctagaaatgttcttgttcttgagatagggtattgctcttttgtccaggctagagtacagtggcacaggcatggcttaccacagccttgacctcctggacttaagtgatccttctgcctcagcctcccaagtagctgggactacaggcatgtgccaccatgcctagctaatttttgtatttttattttgtagagatagggggtctcactatgttgcccaggctggtctcaaactcctgtgctaaagcaatccttcctcctcagcctcccagagtactgggattataggtatgggctaccatgcctagccccagaaatgtttttttaatgaggtttttatagcaccactctgttacacatgccacatttgctttcgttttatgtattctaatgaattgtatttgtaccttatacagttttatcaggaaggaaggaattttttttttcttgagacggagtcttgctctgttgcccaggctggggtgctgtggtgcaatctcggctcactgcagtctccacctcccaggttcaagtgattctcctgcctcagcctcctgagtagctgggattacaggcatgtgccaccacacccagctaacttttgtatttttagtagcaatggggtttcaccatgttggccaagctggtctcaaactcctgacctcctgatccgcccacctcagcctcccaaaagtgctgggattacagggcatgagccaccgtaccaggtggaagtaatttttaagataactaagttaaattgtttcactcctgatggaactaaaacatgtaggtcatgtgtaaataatttttttttctttttttaagataaggcctggctctgtcacccaggctgggatgtagtggcacgatctctgctcattgcagcctctgcctcctgggctcaagtgatcctcgcacttcagccttcccagtcgctggaactacaggcatacacctggctaagttttgttaaattttaatcattaaaactcatctttagccaagtgcagtggctcatacccctaatccaagcactttgggaggctgagatggaaggattgcttgagaccaggaattcaagaccagcctgggcaacagagtgagaccccatctctacaaaaaattaatgaaaaaaaaagccaagcatggtggtgcacacctgtagtcctcgttactcaagagaatgagatgggaggatcactttagccgaggagttcgaggctgcagtgagctgtgattgcaccactgcactccaaactaggtgacagagcaagactccgtcacttaaataaacaaacaaaaccttatctttaaaactaaaaagttattttaaaataggattttgttttgtatcctatgcaaggtttttacttttgtaacatgtttttttttttttgtttagtgtctaaatatgaataaccaggatgctgctgtttactcacctttttagatctctagtttctaggaatttacaaccaaaagttctgaaagaacatgctcaaaaccaacacctttttttggttgcaatccagtaagtgccaggagggtctgaatctagttccctgcaaactccctttccaggttacattttaaataattcaacctccgacctagttgtccatttgagatggaagaactaaattatttaactcatacaaagcattgaactagattctgaagggaaacagcaaagtatagtacctgccctgaagaagctaatttatatagtaggaaaggtaaggcatatactatgcaagtaaaataaaaaacaggcaaatgcatgccaagtggctgtgcagatggtacagtgctctaaaacagagtttctcaacctcagcactaatgacattttggatcaaagaattctttattgtggaggaccgtcctgtgcatcataaaatgtgtagcaacatccctggcctctacccggtagatgccaataacacctccgccacccacccctcagttgtgatgatcaaaaatgtcagaggcaaatgttgagagggtgagtagaccagcatgactgcctgaaagagttcacgtaaagaagtggtggactgtagacaacacatgggccgggcagtggggagcttgacaggcagagctgtgaaggaccagggctcagcgcctgagaagtggtgcttgtcagtgcctgtgaatgaagcttgttagacctgctaggaagtttgtgagtaaggtcaacacagtccttcagcagtcttaaaccatttcttttccctgtttttgcctcattgtaggggatcagacaacaccatcttctttacaacctatgcaaatggctcctgcagtaagtatattttagtcccagaaaataatgtcctgttgtccattcatggtagaattttctgggttgtgcaaggtagaaacataaaaggtccctccattctcctgaaattgtaatagctaaagaaagctgttagtggttattaaaatcttgaaagtaaaaccaaattactacactacccagatcaagtaaaaatataggctatacgtagaggcaatatttaatcttagcaatgtatttatgtaaagcaaatattatgctacagacaatagcaaattgaaatgcatggttatttctacaggaaagcaagggttgtgtttgtgtttgcagaagcagctggcttcccaaactctgacacaacttcaaaacagaaagcaaaatctttcatttactcattcaacagatatttgagcacctgctcatatgctaaacactgttctaaggatggagagaagaaaacacccaaggcctgccctcatggaacgtaaattcaggaaaaccaaaactccagctgggcatgatggttgcatgcctgcagtcccagctacttgggaggctgagacagcaggattgcttgagcccagaagttcaaggacagcttgggcaatgtggcaagacccccagcccttaaaagaaaagaaaagaaaactcaagacaatttaaaataggataagataattccaaactgtgccatgaagaaagtaaagcagcatgctgtggcttttttttttttttttttttgagacggagtcttgctctgtcgcccaggctggagtgcagtggcgccatctccgctcactgcaagctccgcctcctgggtttacaccattctcctgcctcagcctcctgtgtagctgggactacagacgcctgtcaccacgcccggctaattttttgtatttttagtagagatggggtttcaccgtgttagccagggtggtcttgaactcctgacctcgtgatccacccggctcggcctcccaaagtgctgggattacaggcgtgagccaccgcacccgtctgctgtggctttgatgtttttatgaaacactgtattagaattaagtaatctcttacctttccattgagcataaaatctgaaatgtataaaataaaacacaaatatgaattgcctatatctaatgctgtatcactttttttttttttttttatgagacggagtcttgctctgtcatacaggctggagcacagtggcgcgatctctgcccactgaaaactctgcatcccgggttcacgccattctcctgcctcagcctcccgagtagctgggactacaggcgcccaccaccacgcctggctaaatttttttgtatttttagtacagatggggtttcatggtgttagccagtttggtctctatctcctgacctcgtgatccgcccacctcagcctcccaaagtgctgggattacaggcatgagccactgcgcctggccgctgtatcacattttatgttgtcacttgattaacatgcatagtatttaaaatgctttagctataaaattaagattgattctaaaacaactgctttgaactaggctcaccagtaagtgaaattttaattaatattttgtcttgtttaagaagctgaccttggggctctggaattatggagaacttcagacttgggaaaaagcttcaaaactattggtgtgaaaatctactcatttggtcttgggggacgtttcctttttgcctctgtgatggctgataaggtaggtattgccctctcccctttggggtctgatatttctgcaaatatctctcgtatagattctctttattacttggcattgacagagtttcttgtctttttgttatatttagtaccactgtgagtgttaggggtgattctttttgcaaaggctgattgactgataggctactctgcactttggccttctgtcaccatgtgaattctgttatcttttttgggattttttttttttttgaaacaaagtctctctctgttgcctaggctggagtgcagtgacgcaatctcggctcactgcaccctccgcctcccagattaaagcaattcacctgcctcaacctcccaagtaagtgggactataggtgtgcaccaccacacccggataatttttgtatttttagtagagatggggttttgccatgttggccgggctggtcttgaactcctgagctcaggtgatccccccgcctcagcctcccaaagtgctgggattataggcgtgagccaccgcgcccggccaggatattttgatccagtgtttgggcattactcagacaatggacatctgtagaaagcaatctcttgctcattatccaagaataaaaatacaattagataacattaattgagtgcctactatatgctaggtactattctaagtcccaggaatatggcagtgaacaaaacagatgaatttcctgctctcagaacttatgtttaagggggaaaataaatactcaaaaaataaatatgaaggctgggggtagtggctcacacttgtaatctcaggactttgggaggctgaggcgggtggatcccttgagcccaggaattcaagaccagcctgggcaacatgacaaaaccctgtctctacaaaaaataagaaaattagccaggcatggtggcatgtgcctgtagtctcagctacttgggaggctgaggtgggaggatcatctgagcctggggaggttgaggctgcagtgagctgtgatcatgccactgcattccagcctgggtaacagcacaagaccctgtctcataaataaatataaatgtgagtgtgtgtgtatcaggtaataattaaagccgcgaagaaatctaaaacagtaagaaaacaaagtgtcaggagtcagtgctcttttagacagactgtttaggatgggctctctgagacctaagtgaaacaagaacttgaccatgtggccatctgggaggagagtaggatgaggcacagccagtgcagagaccctcagactggagcatgcccgatcaatccaagcaaaggggagaagggcagtatggggggagcacagagaataatagtccaaagtggggccagagggggcctggccagaccttatgtggcctttgtagatcacaattaggatgtgggccttactcttcttgtgttgggagctgtgagagggttgtgagcaggggaatgctgagatttggcttctcttttgaagaataggtgtgcgcagaacagcctgacaggggcacgcgcagaagcagcgggagcgccgggagctctctcaggagtccaagctggggaggtggtagcttggccaggctcttggcagtgggtaagggggcaggtggtgagactgtggatacatttttaaacatattactaataggacttgctgatggatttgatgtgggttgtgagagaatgagtggaatcaagggtacctttaagagtctgtatcctgagcgcctgaatggtggtgccattttctaggatggcaaagactgcaggaggagcaggttgtgggagaaaaatcaagaattctgttttgggccgagcatggtggctcacgccttaatcccagccctttaggaggctaagacgagcaaatcacttgaggtcaggagtttgagagcagcctggccaacatggcgaaaccctgtctctactaaaagatacaaaaaattagccaagcgtggtggcgcaggcctgtaatcccagctactcgggaggctgatgcacaagaatcaactggactgtggagacggaggttgcagtgagccaagatcatgccactgcactccagcctgggaaacagagtgagactctgtctcaaaaaaaataaaaaaagaattcagttctgcatatgagacacctattagatatctgaatggagatgtcaagtgagcatttgggtatatgaatctccctaattgctatggtatttgaagccatgtgaccagatgataccttctggaggagtaagtaggtagcaaagaggtgggaggactgagcaagccctgaagcacccacaactgaaaggttggaaagagaagaatccagcaaagaagactgagagggagggatccgggaggaagagggaaatcaggaccattgagttaggtgcattctgagaattatccactggagttggcgacgtggaggttgttggtgaccttgacaaaagcacttcattggaagttaaaaggccgattggagtgacttcagtgagaactagagatgaagaagtaggcagtaaatacagataactctccaaagccattttgtaatatcctcccaataaccctgtgagctaatagtattacctcagttttataggtgaggaaattgagtaacttacctagggtcaaccagtagtggaatcctgctggaaatcagactcaaattccagaggtagcttcctatgctatactgcatatatggtataatgctgtctacagaattaaaattttgcgatgtacttttgaactctggtttctacctacagtaatgagaataagattcaaagtaatgagacatgttggtataagttgaataagttaaatgtacttatcactgaactttaaagagtatttaatctaaggaaacaccgacattaattctcatgctcaggctaactgtatctagtagcgatgttaagctatttatcagagagttaagttttaattgctttcttaacagactcttttcgtcagccaaatactccctaatcagtttccttatagtaatttccgactctccttggcctctttccagcccatgcctgttcccttcatcctggctattctgagagacttccttagagagaaggcgtgttacatcttgacttcccttgtgatctcccaaacatgggtttcatctctattttctagacccttaagtatataattctttaaattatgcaagcactgtatgaaagggaccaaacttttatgcagtagtcatcattttaagaattacctgagtgtatgcaaatggctataaagccaacctaatgttctcatcaaatccttagctctactgaaggaggtggactatcagagaccaagactatactatcttacagggccgcatcagaatgaagaattcaaagagaaagccaagatagccttacttagaatagaatggattgttcctttacctagaactttgctaggggctagaaattgctttggtaattggtactatgacctttcattcatagcaatcgtacccagtagagaaatggataatagtagttatttaaaacattcttgttcatgtccctgcccatggcatcttggcagaaaattagaaagataaatattgacatctgttctaggatacaacaagaaggatccacgtttcaacagatcaaggggacacatggagcatggcccagctcccctccgtgggacaggaacagttctattctattctggcagcaaatgatgacatggtattcatgcatgtagatgaacctggaggtaagagctttttagtggcttacccttaaaagcatagagatgtaagctttgagcagcttttatttttacatgtcgagttttcctcttacccctagaaatttattagtgccctactgatttttctgtcctagagatcatctgtagcccctgttaaaaatgccacttgttttggtagcttgtgtgtatgttcaggtgttctagagcagagctgctcaaagaatggtcagcagactggtactgatttgcaaactatctgttaccagataagtacagaaattgagagcaagagtttggaaactcttctagccttttgacaaagcaattttatgtctgttggatctaatgacaaaaaattaggggtttatggctgggcgcagtggctcacacctgtaatcctggcactttgggaggctgaggcgggcagatcacgaggtcaggagatcgagaccgtcatggctaacacagtgaaaccccatctctactacaaaaaatacaaaaaattagctggccgtggtggcgagcacctatagtcccagctactcaggaggctgaggcaggagaatggcatgaacccgggaggtggagcttgcagtgagctgagatcgtgccactgcactccagcctgggcaacagagcgagactccatctcaaaaaaaaaaaacttaggggcatatattttgtttaccttttcattttctaatgattagtttttattatgttttacaatagcatcagtttgtgacatattggaaatcttacaaattgatccttcactacagatagtttgagaagcattgttctagagtgtaaagatttaaaaatatgtaaactaaacatagggcctctcccaccaaaatatatctgggaagagtttttctgtagcatctgattaatttattcaacaaactcagcatctgccatattccaggcactaagctgggtactagaattaaagatgggcataaagatcttgagtatcaaaccttgattaattctcaggtctgagtcttcatcatgaagagacacaattggctactgtgtttggtcaggcaggtaatcgtgtgtggttagactattgtttttctctttggttccacccaagacttagtatttttgtttgtttgcctatttcagacactgggtttggcacaatctttacctcagatgatcgaggcattgtctattccaagtctttggaccgacatctctacactaccacaggcggagagacggactttaccaacgtgacctccctccgcggcgtctacataacaagcgtgctctccgaaggtgagtctcggcatcgcagtggtgtactgtgccttcagctgcatgtggtaggatttgaggcttcggggaacctgtcccctagtaatgttgcttgccaagccaacaagcctacccatgtacatttttttgtttccttttttttttttattgtggcaatatgtctaacaaaatttgcagtttttaacattgtatagggtacaattcagtagcattaagcacagtcacaatgttgcacaaccatcacccctatttccagagctttttcatcatcccaaatagaaattgcaccaggtgcacaagcactcctcgttccccacccagtcacccagctctggtaaccaacattctattttatgtctcaatgaatttgcctattctaggtagctcatgtaagcagaatcatacactattggttgttgtatgtctggcttctttcattttgcatattgtttcaaagttcagccacattgcagcatgttttagaatttcacttattgctttttaagactagaatatattcctttgtttgaatatagcaccttttttttctttttctttcttttttttttttttttttgagatggaatcgctctgttgcccagactggagtgcggtggcgtgatctcagctcactgcaaccttggtctcccaagttcaagggattctcctgcctcagcctcctgagtagctgggtttacaggtgtgtgccaccacacccagctaatttttgtatttttagtagagatggggtttcaccatgttggccaggttggtctcgagctcctgacctcaggtgatctgcctgccttggcctcccaaagtgctgggattccaggcgtgagccaccatgcccagcctgttttttattcctccattgatggacacttaggctgtttccattttcagctcttatgaatactgaatgctgccatgaacattgttatacatgtaactgtttaagtcaatgctttcagttcctttggggatatatgtaggagtgaaatagcaggatcacatgataattccatgtttctcttttggaggaaataccaatgggttttccacatggttgtaccattttacatgcaatacaaaaggattccaatttctctacttcattgcaaacacattatattctcctatttttgtaacagcactcctaatgggagtgaaatactatctcgtttatagctttgatttgcatttctctaatgactaaagatgttgaacaactttttatatgctcgttggccatgtgaatatcttcgttgaggaagcttctaattgagtcctatggatggcagtgatttttaaagttgtgttcactgatgctcctctggtgcttatgacaatttttgcacatagtagatgttcagtaatacttgccaaatgagcctacaaataaacggctgaacagccttgcactcaaaaccatacagtgaacacacttgaaaaacatttaagactgagccttaagtatgataggcaggaatggggagggaagggcattttcagctgaaggaagtacacatgcaaagtatcagagtcgctgggcgcggtggctcacacctgtaatcccagcactttgggaggctgaggcaggtggatcatgaggtcaggagatcgagaccatcctggctaacacggcgaaaccccgtctctactaaaaatacaaaaaattagctgggcgtggtggtggacacctgtagtcccagctactcaggagactgaggcaggagaatggcgtgaacccgggaggctgagcttgcagtgagccaagaccatgccactgcactccagcctgggcaacagagtgagactgcatctcaaaaaaaaaaaaagtatcagagtcacaggagggtttggcctggtggtggtggtgagacattgggtgatgccgggggaggagggatcaggaggacagtccagcagggacagaccaaacccatgcaaattcaggctccctattttaaataagttatgataaatagaactaggagtgaggagagcaacaacaagtagaaaaaaagatgttttgaatctatgttgtttaggtaataaagtgagtcttctatattgggagatgtaggcctatgagaataagcctgaatttgacccacttcctgctcttcttggtaagggtgcatagcagtagccagccagggttcttcttgtgttctactcaatggctctacagagctcacctttccttaggcccagttatacaaataaacacattagtctcaagtacaaagactcttaaagtgtcattttatttatatatttatttttcggtaggaggtaggggctgtttttattttgtaaagtcctggcagtggcctacaaagtcctggagatatggctcccggtttcctccctgactcacctccagcgcttctctttccatctgccctactctcatcatactgtccacctgccagttcttaacacagcaggtacacatgcacatgcacagattagagtttgcaccgacagttccctctgtttctccagatgtccgcaaggctgatatgtctgtactcatgtattactttttgaatgagaccttccctgactatcctattttaaattccagtttgccctcgcctcctcacacacacactatcaatccacctttagctgcgcaagtattttccatagtacttattttctagcatactacataatttacttatttactatgtctagtgcttgtctcgccctaccggaatgtatttttcacaatatttgtctgtttagttcactgatacattaccaaaggcttacaaaagttttcactctgggtgtagtggctcatgcgtgtaatcctagcactttgggagaccaaagtgagtggatgacttgaggtcaggagttggagaacagcctggccaatatggtgaaacccagtctgtactaaaaatacaaattagccagacctggtggcatgcacctgtagtcccagctactcaggaggctgaggcaggagaatcgcttgaacccgggaggcagaggttgcagtgagccgagatggtgccactgcgctccagcctgggtgatagaacaagactctgtctcaaaaaaaagagttttcacatagtaggcactcagtagatgtttgagtaaatgaattttccttcatttgcttccttgtttgctttttcttagaaaactcaaaggagcccaggcacagtggctcacacctgtaatcccagcacttcgggaggctgaggcgggtgaatcacctgaggtcagaagttggagaccagcctgaccaacatggtgaaaccccgtctctactaaaaataccaaacattagccaggcggcaggtgcctgtaatcccatctactccagaggttgaggcaggagaattgcttgaacccaggaggcagaggttgcagtgagctgagattgtgccattgcactccagcctgggcaacaagagcgaaaatccatctcaataaataaataaataaataaaaataaaggaaaactcaaaggatattatctctgaatttaaaacctgcaacagtttatatcattgaaaataagaatggggaagagacgagggaaaaaagaaaggtacaagaaagataggatatggagtagtggagagacaataattcagagttattaatccagaactctgcacacttactgaatctcaagagtccctaaaacaaggctagtgtcatggccttgtccgaataaagattaggatgccttcctgtggtagattttacttcccaggcaagcaaacagggaaggagaatggacatcagcctgttcatgttggtaaggagttaaaaggtatttttttcaaagagtatcagtcctaaataatttgtggaattaattgggtcctgttcaggtgggacaccttcttcaaatatcttttttttttttttttctgagacaagagttttgctcttgttgcccaggctagagtgcaatggcatgatcttgaccccctgcagcccccacctcccgggttcaagtgattctcctgtcccggcctcccgagtagctgggattacaggcccccaccaccacacccggctaattttttgtatttttggtagagtcagggtttcaccatgttggccaggctggtctcaaactcctgacctcaggtgatccacctgccttggcctcccaaagtgctgggattacaggcatgaaccactgcacctggcctcaaatatcatatttaaagtgctgctccgttaccaacaatgagccgttttgtgaggtgttacaagcttgtcggactccaggtgctatacggtctcaggagagagtcatgcagtgataaagtgaatctaacctaggtacttccagtgtatcagacttaatgttgttctcttcccattcagataattctatccagaccatgatcacttttgaccaaggaggaaggtggacgcacctgaggaagcctgaaaacagtgaatgtgatgctacagcaaaaaacaagaatgaggtttgtttactctaatgtgtgcagagcacagcagcctttccattgtgctctgatcagtgccagccttttggatgatgaagtttcatacttgattagggtcctcagcataaggtggaaagtttggagatgagagtaatgaaattaacttagaatttcggaagtaagaactgaggaaaggtcaaaatactatggattatttgtcttttaaagaagaacaaaactgagggctaacaatttgtggttcactaaactgctgaattaaaacaatggccagaggggctggtgattagttaggggtcagcttggcagctgggagactttagaggttctgcaagaagccgcagctccaccattcataagcagtgcacagccttgctcatcggaaacaagaggaagaggatctggccttcctttccctctgcctaggctcccatggccagtgggtggtgacctgcttagtgcacagggacgcaaggcagcccctgttgcagagagtttgggaaccggaactacgcaggtgtcactccaggagatctaaagcctggagtgacaggccttggcaggcaaggaaaaaagggaaaaaaaaaacaagctttttatctggctctcacagaatgtgagcttgggggacagctggagttccttcagaaactgaagtaccggtagttcccgaaccctccttagtatctgtatcactgacatacactttgcttttctaggtggggttttctgttttgttttaagagcctcaaatgatgagagaaagtgtgccttcctacaccaccccaacacactctcctcctttacactcgcaagcactgttgcatctacccgactttctgacccctgtttcccatctgtctgccttggccctgcccagccactgccctcctcaccttaagcctgggcaggttcagtggcctctgtgtttgccttgctgcctgcaggctccccatgcctctccctgcccagcgctcgttgtggctctcttggcctgtgatgaatggcctctgtccctgacctggtgagtaaaggttctctcctggtgggccctctgccaccttttcattgtcattgctcaccttatccctgacacaccccttgtaaccaaccccctgccattccctgaacacccacactattcctggaggaagttgggcctgtctcccccaaccccagttgactaggattggtggaaattctaaccaccctcagtgaccatccagttagagctaatcattcctcttcatttcattaatagtttgcttgaacctcttctttttaaaaaaatagtagtaaaatatacataatataatatttattatcattttagccattttgaaccctttttagagtgttaactttcattcggctgtatacctcaactagtttctgacaatacatttcttttttccccagctttatggaggcataattgacaaaaattgtatatttacaatgtacactgtgatgttttgatatatatatacattgtgaaatgattatcacaatcaagctaactaacatatgcatcacctcacaattatcttttgtgtgtgtggtgagaacatttaggatctattctcttagcaaacttcaagtaacaatacagtattctagactgtagtcaccatgtatattttccctcagaagcttattcatcctgcataactgaaactatgtaccctttggccttcatctccccataccccaagccccaaaagccaccattctactccctgcttctgtgagtttgacatttttacattccacttataaatgagatcatgcagtatttgtctttctgtgctgggcttattttacttaacataatgcctcccaggttcatgttttcaacagatgacaggatttccttcttttttaaggctgaataatattccattttctttgtccattcatccatctgtagacgcttaggttgattctgtagcttggctattgtgaataatactgcagtggacatgggagcacagatatcttttggacataccgatttcatttctcttgaatatgaacccagaaatgggattgctggtagttctgtccttcattttcttttttttcttttttttaaagacacagtcttgccccgtcaccaaggctggagtacaatagcacgatcttggctcacttcaacctccgcctcccgggttcaggtgattctcctgcctcagcctcccaagtaactgagattacaggcatgtgccaccacgcccggctaattttttgtgtctttagtagtgacggggtttcaccatattggccaggctggtctcgaactcctgacctcgtgatccacccgcctctgcctcccaaagtactgggattgcaggcatgagccaccatgctcggccctgctgttattcattttctaggaaaatttcttacggtattgcataatggctgtaccagtttacattcccaccaacagtgtacagggttcccttttttccatgtccttgccaatacttaccttttatctctttgataatagccattctaacttgggtgagataatatctcattgtggtttttatttgtatttccctcatgattagtgatgttgggcattttttcgtatacctgttggccatttatatgtcttcttttgataaatggctgttcaggtcctttgcccattttttagttggattatttgttttcttgctactgatttgtttggatattaaccccttataagatgtatggtttgcaagtattttctcccattctgtgtattgtttgtcttctctcttttggtggtttcctttattttgcagaagctttttagtttgatacaatctgatttgtctgtttttgttttttgttgcctgtgcttttagggtcgtatccaaaaagccattgcccagatgaatgtcaagaagcttttttccctatatttccttctagtagttttacagtttcaagtcttacatttatgtctctaatccattttgagttgatttttgtacatgacatgaaataaggattgaggccaggtgtggtggctcacgcctgtaatcccagcactttgggaggccaaggagggtggatcacgaggtcaggagatcgagaccatcctggccaacatggtgaaatcccgtctctaccaaaaatacaaaaattagctgggtatggtgatgtgcacctgtagtcccagctacttgggagactgaggcaggagaattgcttgaacctgggaggcggaggttgcagtgagccgagaccgcaccattgcactccagcctggcgacagagcaagactccatctcaaaaaaaagaaagaagggttgaaatttattcttctgcatatagatattcagttttcccagcaccgtttatttaagaggctgtctttttcacattgtgtgttcttgacacccttgtcaaagttaagttgacaataaatgcatagatttatttctgtgctctctattctgttccattggtctatttgtttttaacgccagtaccatgctgttttgattactatagctcaataatatattttgaaattaggtagcatgatacccccagctttgttctttttgcttgcaattactttggttattcagggtcttttgtggttccatataaattttaggattttttctatttctgtgaaaaataccattggaattttttttaagaggcaaagtcttagtctgtcaccctgactggggtccagtggaataatcataggtcactgcagcctcaaactcctaggctcacacaatcctcccacgtcccaagtagctaggtgtgtaccaccacacctgtctaattttggtactttttggtagagatggggtttcaccatgttgcccaagctagtcttgaactcctggcctcaagtgatcctaccacctcagcctctcagctgggattacagatgtgagccaccacaactggttgttttgttcttgatgtaccttaagtacctaaaacagtgcctgaaatatagcaggtactcagtaaacatctgatgaattaacgtgtttggattgttctgtgttgtcacagaaagagggttatttataaccatataatagtaaaaaaagacagtcaaaataataggcattacatttaatctgtagatcactttgggtagtatggacattttaacaatattaattattccaatccaggaacataggatatatttccttttttttttttttgtcatcttcaatttctttcatcaatgttttgtagtttttagtctttcacctccttggttaaatttatccctaagttttttttgttttttttttttaagtttttttcttgtactattgtagatgaaattttcttgatttcttttttggatagtttgttgttagtgtatattgtatttttatatgttgattttatattctgcaacttttttgaatttattagttttaacatttttggtggagtctttaaagattaaggatttttatatataagctcatgtcacctgcagacagacaattttacctctttctttctggtttggatgctttttgtttttatttctttttcttacttaattgctccagctaggacttccattactataatgaacagaagtggtgagaatgggcatctttgtcttattcctgatcttagaggaaaaggtttagcttttgactgtttgagtatgatgttagctgtgggcttgtcacatatagcctttattatgttgaggtatatcccttttatacctaatttattgagagtttttatcatgaatgggtttattatgttgaggtatatcccttttatacctaatttattgagagtttttatcatgaatgggtgttgaaatttgttaagtgctttttctggatttatcaagatgatatgatttctatccttcattctattaatgtggcatatctcatttattgatttgcatatgttgaaccatccttgcattccacagatagatcctacttgatcatggtatataatctgtttaatatgctgttgaattcagtttgttagtattttgttgagaatttttgcatctatgttcatcagagatattgacctgtaattttgttttcttctagtgtccttgtctggttttggtagcagggtaataccacccttataaaatgagtttggaagtgttccattctcttcaatttttggaagagtttgagaaggatcggtcttaattcttctttgaagaaagtttgctagaattcaccattgaatccatcaggtcctaggcttttcttgttgggaggtttttgattactgattgagcctccttactcatattagtatgttcagattttctatttcttcttcatgatacagtcttggtaagctgtatatgtctagaaattcatccatttttttctagattatccaatttgttgatgtatagttgttcatagtagtctcttaggatcctttgttttttctgtagtataagttataatgtctcctcttacatttctgattttacttgagtcttttctttttttcttattctaattaaaagtttgtcacttttttatcttgtcaaaaaaaacaactctaagtttcattgatctttcctgttatttttctaacttctatttcagttatttctgctctgttattatttcctcccttctgctaactttgggcttagtttgttctttttctagttccttgaggtataaagttaagatttttgagatttttcttttttcttaatgtagacatttatcaccataaactttcttcttagaactgcttttgctgcatcccttaagttttaatatgttgtgtttcccttttcatttgtctcaagttttttttatttccctttgactcattggttgttcaagaatatgttgttcaatttccacattcatgtgaattttccaattttcctccctttactgattctagtttcataatgttgtagttgaaaagaacttgaggccaggcacactggctcacgcctataatccttgcactttgggaggctgaggtgggtggatcacgaggtcaggagttcaagaccagcctggccgacttggtctctactaaaaatacaaaaaaaaaatagctgggcgtggcagcgggtgcctttaatcccagctactcaggaggctgaggcagagaattgtttgaacccgggaggcggagcttgcagtgagccaagatcacgccactgcactccagcccaggtgacagagtgagactccgtctcaaaaaaaaaagaaaggatacttgaggtgatttcagtcttacatttgttaaggcttgttttgtggcctaacatatgcactatcctggagaatattctctgtgcacttgaaaagaatgtatattctgctgctattggatagaatgttctgtatatgtctgttaggtccatttgtatggtgttgttcaagtttgctctttccttactggttttctgtctggatgatctatcattgaaagtaagtattgaagtcccttactattattgtattgtcgtctacctctttcttcagttctgttaatgtttgctttatacatttaggtgctctaatgttgagtgcatttatatttaccaatgttatatgctcttgatgaattgacacctttatcattgtgtggtaactttctttatcccttgtgatagttttttacttaaagtctattttgtctgatataaatatagccacccccggctggacatggtggctcacgcctgtaatcccagctctttgggagaccaaggcgggcggatcacgaggtcaggaaatcaagaccgtcctcactaacatggtgaaaccctgtctctactaaaaatacaaaaaattagccgggtgtggtggcaggcgcctgtagtcccagctacttgggaggctgaggcaggagaatggcatgaacccaggaggcagagcttgcagtgagctgagatggcaccaccgcactccagcctgggtggcagagcgacactccgtctcaaaaataaataaataaatatagccacccctgctctcttttggctaccatttgagtggaatatcttttttttatctctacttttagcctatgtgtcctcttacacacataggcctttacagatggcttcagcaggggaagcccttaccaggtagctcatccagagattcttgggggggcaactgacaggttctgcaggtaggtgggcctggtgccttggtctttgggggtagcttggaggctgggtcctctgcggcaggcctggcagtggaatccactagggtggaccttttaagtgggtcttcaggtgaaacacctggagcccggtaagtagatctgcaggagccagcctggtgcaggggtgggccctaccctgagcctgcaggggctggcctgatgcaggggtccacttggctgggtctgtgagagtggacctggagcctttaagtttcttaaagacaagactggatcttatttcgaaccaccatcacatcttgtacggtgtctgaagtatatcaggtgcttatcaagtttatttgtgtagagtcagcctttaaacattgttcagaggctttgatgtaatgcaaaaataaaataaacggagacaggttacccatgaggaaatatttgcttccagatattgtagtacatttcagaatcaagagtatgccactgagctacaactcaacaacaaacaaatagcctgaataaaaaataggcaaagaacttgtatagacatttctccaaacacaatatagaagtgactaacaagcatataaaaagaggctcagcatcactaatcattaggaaaatgcaagtcaaaaccacaatgaggtatctcctcacacagattagggtggctactattttaaaaagtgttggcaaggatatggagaaattggagccctggtgcacttttggtgaaaatgtaaaatggtgcagctgctacagaaaacagcatagtagttcctaaaaaaaattaaaaataggattatcatatccagcaattccacttctggattatacatccaaaagagttgaaagcaggttctcaaaaagatatttgtacaaccatgtacatagcaacattattcatgatagccaaaaggtagaagcaacttgtgtctattgacagataaatgagtaaataaagtgtggggtgtgtgtggatacagatacacacacacacttaaataatatttagcctttaaaagggaggaaattctgacatatgctattaatacaacatggatgagccttgaggacattatgccaagtgaaataagtcagacacaaaatgacaaatattatgtgattctacttatatgagctacctagagtagccaacacaaagtaatatggtaaaaatcatagaaaacacaaagtaaaatggtagtttccaggagctgggaggaggaagaagtaggtaacagttgcttaattggtgattgtattttataagatgaaaaagtttagagatgtggttgtacaacaatgtgaatatacttaacactactgaactatacacctaaatagtaaataaatagttaagatagtacttttacctaggtctgggttaacttaaagctttattggaagggttttgtgaaatgattaaagaaattgtatctttatctttggttctaatctacgcttttgtttcttttcacagtgcagccttcatattcatgcttcctacagcatctcccagaaactgaatgttccaatggccccactctcagagccgaatgccgtaggcattgtcattgctcatggtaaggaacctcccactcaccacctccaactggaacagtggctgagcacactgaaactcagccaacaggctttgaacgctctgagaagaaacaatgtggatgcttttacattcctttggtagaatttcttacattataacaacactttgggtttttcatcaacttttatcagagagagcaacacaacttctccgaagtttttggtggttttctttcctactaaaagaggcttgatgggaaataatcagaataaattcattcaacattattctctctggagtaaatgctctctgtttcattgcctatttgtgctaatgagatggtggattatattgcaggtagcgtgggggatgccatctcagtgatggttccagatgtgtacatctcagatgatgggggttactcctggacaaagatgctggaaggaccccactattacaccatcctggattctggaggcatcattgtggccattgagcacagcagccgtcctatcaatgtgattaagtatgcatgaactcatctcacacacactgtctgcatagagggcaactgggggaatgttcttcagtttattggggggatgttacagactataattcaaatttttgcatttgacagaactatagataagattctagaaaggattgcttttaattggcaactcagattaaatggcattgttttttcttctgactgcctttagctaacatatttgtcacttcttaaaaacattctttctgggttgggcgcagtggctcatgcctgtaatcccagcattttgggaggccaaagcaggaggatcgcttgaggccaggagttcaagaccagcctgggcaacatagggagaccttgtcccttgtctctaatattatttcaatcaaaaaaaaaaccatatatatatatatgtgtgtgtgtatatatgtatgtgtgtgtgtgtatatatatgtgtatatatatgtatatatatgtgtatatatatgtgtatgtgtgtatatatgtgtgtatatatatgtgtgtgtgtgtgtgtatgtatatatatatatatatatatatatatatatatatattctttctgtctacaaaattattatggaagcgttcaagcactctagattgaaaatgacttcttaagtacaagttctgagctgggtgcagaggcacacacctgtaatcccagcactttgagaggccaaggtgggtggattgcttgagcccaagagttcaagaccagcctgggcaatgtgacaaaaccctgtctccacaaaaaaaaaaaaagaaaaattagctgggcgtggtggtgcacgcctttggttccagctacttgggaggccgaggtgggaggatcacctgaggaggtcaaggccacagtgagctgagatcgtaccactgcactccagcctgggtgacagagtgagatcccgtctcaaaaaaaaaaaaaaaagaagtataagttcttataaaataatttgattgttatttatttatttatttattgagatggagttttgctcttgtcacccaggctggagtgcaatggtgcaatcttggctcactgcaacctctgcctcccaggttcaaatgattcttgcgtctcagtcacctgagcagctgagattacaggcatgcaccaccacacccagctaatttttgtggttttttgttttgctttgttttttgtgttttggtagagacggggttttgttgtgttggtcaggctggtctcgaactcctaacctcaggtgattcacctgcctcagcctcccaaaattctgggattacaggcgtgagccaccacagccagccttaaatttattaatttaaaagccagttcttacctgggtcactctcctgtctttcctttgaaataatttctgtaaaggtatttactttcatattctctctgaacaaatctcttccttatcctgttaactacagacgttaaaagttaaaatagttggctgggtgcggtggctcacgcctataatcccagcactttgggaggccaaggtgggaagattacctgaggtcaggagttcaagaccagcctgcccaacatgtgaaactccatttctactaaaaatacaaaaattagctggtgtggtggcacgttcctgtaatcccagctactcaggaggctgaggcatgagagagaatcacttgaacccaggaggcagaggttgcagtgagtcgagatcgcgccactgcactccagcccggtgacagagtgagactccatctcagaaaaaaaaaaaaaaaaaaaaaaagttaaaataattgtcagccaataagccaccacctaaatgtaaggggttttgtctggtttgggatcaaaaagccagagtggttttctgaattcccagtacacattgatcctctgtcatgatctttctgtaattgtggtttcaggttctccacagacgaaggtcaatgctggcaaacctacacgttcaccagggaccccatctatttcactggcctagcttcagaacctggagctaggtccatgaatatcagcatttggggcttcacagaatctttcctgaccagccagtgggtctcctacaccattgattttaaagatatccttgaaaggaactgtgagtgtctcctttgaccttttctaccagaaacttccaagtccattttcctaaataatcaatatggtttaatttcttttagctggaatcagcagatgcatatgcatatgaaagaggattgacagatgttctgtgtgggtgattaagtaatggaatggcatcagaaaaacaacctcctcctcggtgctagcactgtccaggagaaatatgagggccacatatgtcatttaaattattctgtcagctatattataaaagtaaaaagaaaggtgatattaataaaataaacttaatatatccaaaatattatcatttcaacattaagcaatattaacggcccagaaaataggttcagaaaaggattcacaatgttcattgcacagtgtttccaaagtgtcacaagcaaaccctaacctcactgccaaacaaagatgctggcattatgctaagaaataaggggaatggccaggcgcggtggctcatgcctgtaatcctagcactttgagaggccaagataggcagatcacgaggtcaggagttcgaaaccagcctggccaacagggcgaaactccatctctactaaaaatagaaaaattagccaggtaaggtggcgggtgcctgtaaggtggcgggtcccagctagttgggaggctgaggcaggagaatcatttgaacccgagaggaagaggttgcaatgagccaagatcatgccattgcactccagcctgggcgacaagagcaagactctgtctcaaaaaaaaaagaagggggataaagaaaaacactggaaaaaggatgacctgtagttaagtgttggcataatacaataaaagacttaacgtttctagacactctagttgaatttggtcctttgagactgtcgtgggctcatttcctcagcgtgttgatttagtgactcctgaaggagcagacccttggtttggacctcacatttccataagacgttcaaatagatttaatgcctgagggtcgctgtcatctaaatccattcagtagactgtacagaagctggcttgggagttctcccctcagcaggcagctcccagttacccctaaagtgtaatgtcattaaaagatttgttaagcacctgctctttagcaggctgtatgctggtcttcatatcccaaaggataaaacctaatccactgtctttaggtcacagcctttgagtcatttttccagtagatgatacgacatactaagccagaaactaggtttatcagcaatatgcatttgagagccggtcaactgagctagaagtctaggaaaaggatgttttcccttcttccttaagccaatgaacacatggtgaaattgaacctgtgctcttggctaattagtatcttgtcctaaacaaagagctccctggtcctgagtggagaatatctggttgcttacctctgacagctcctactgactgagggcgtaggtgtgcaagacacacacagcagcctcttagtttggaaggaacttgagagattttctatccagcttcccacttcaaaacaaatagggtttttcctaatcctcagatggatgttgagatggaaggaatactcttttggcatgaggaaatgcgattgctagtaggctctagactagtgtaactttttgtttgcaattccaaaatgaaggaataggttgagtcagacattcccactttccctctccttgttcttcctatagcctggcagctgcctcccgtgggtgggttctgtgctgtgtactttcctgaacagtgtgtctctcctttaggtgaagagaaggactataccatatggctggcacactccacagaccctgaagattatgaagatggctgcattttgggctacaaagaacagtttctgcggctacgcaagtcatccgtgtgtcagaatggtcgagactatgttgtgaccaagcagccctccatctgcctctgttccctggaggactttctctggtatcagcattcctcagatttctctgtctccctttccctgttggaggaggtgaaagacagttttcttatagggctaacaaatattgaaatccagccgggtgcggtggctcacacctgtaatcccagcactttgggatgccaaggcaggcagatcacttgaggccgggagttcgagaccagcctggtcaacatggtgaaaccctatctctacaaaaaacaaacaaacaaaaaaaaattagcagggcatggtggcgcacgcctataatcccaagctgctcaggtggctgagacacaagaatcacttgaacctgggaggcagaggttgcagtgagctgagatcatgccattgcactccagcctgggggacagagcaagactcttgtcccaaaaaaaactgaaaccctttcccccaagagtggagaactgctgcttcttatcaaggaagctgaggcacagagaaaggaaaccagaattgttaataatgaaacagatagcacgtattagacagttaattattgggcagaaccagcaacaaacccgagaaatcctgagttgacagtggtgtgaaggagaggagtgatataagaaagaagagaataaaacaaacaaaaatctgtagaaaatgaaaagacagaaacaccaaaagaagtggtagaggagcaaagaggaaattggactttagaaaacagcctgaggccaggcacggtggctcatgcctgtaatcccagcactttgggaggccgaggcgggtggattgcttaaactcaggagtttgagaccagcttgggcaatatgatgagaccctgtctcaaacaaaaaaaaaaacagaaagaaagaaaaagaaaaccccttgacagcatgagctcagcaggcagacactaggttttggatcctctgcaagaatcaaggcagagagatggtggttggtcctgagactagaggtgacatcagtatagtcatggtcccatcaaggtttagaggccatcaaagtggactccctagagttcaggaccatccagacttaaggattagttacttggctggcattgggttagttgattatataagggcatgagtccctggatagcttaaatgaggtaatttttgggcaaataagaggaactgcttcacatcacagatgataatcttttggaacttgttacctgcaaaagatactgacacaagataaaagtagatcctaaaggatttggacaaagaaggcaggcagatagcagccatggccctttatgtcatgttctctcaaatcgcaggtgacagtagaatataggtctgaccaaaatgcaggtctgacccagtgtggtttctcctgcatggctcacacctgtgtgtggcatgattaagggtcctcccttctctgtttcagtaaggatcacctttggtgttccagtagggctggaggtcatcgataaacctgtaacactgtaaagggttctctcctatcctcgactccaaatatgccaagctcttcacctgtgcttctgctcttctccagcagtgccatggtttttcagacctccatgccaccgcacatgatgctccctctgcctgaaaagtctttccacagtccctgcttcacccccaagtctggcgacttcccacttagcctttcatacttagctcaggtatcaccactagatgggctttccctcatctagacccccagagttcccctattacagcacttactaaattttaattactatttataggtttgcttccatcattaagccccaaattccttggaagcatattctattcgataataatatagtaatcatgatagcagctaacatttattgagcactaactttatactaggcactatcctaagcaatttacatttaacctcgcaacagagaggacatgactgatatgcccgtgggaagtggcagagctgggatgtgaaactggcatcccaactccagagcccacatgtttgggaagtgggtcctttatgagtgagaagggtactcacaaaaatccagaaatttctgattgaatacctaaaactcttatccgaatttcctcctgttatttgaggggtgatatcttcagcatcttttgttctccataccctctctcctaaatacttaccaggaaaaccttgaaatacaagcagcatccatatattccccaaatactcattgaataccagctatgcgcaggtaatgaaaagtgataatggaaaatttgttgcaaaacagaaggtgccaggtgccatggggatttagaaaaggaagagaacatttcatgtagagctgaaaggaagtgggtcaaagaaggccttgttgaggaggtggtatgagagctgagcgttgacaaatggtaggatttgaccatgagcatctgggaagaagcagctcatcaatgccagtggactgctgcaagcagagacaccaacagcatgatcaaggaagggtgtcctggaagataggccattggggaagtcacagtagtcatgataaagaagtaagtagggcccagaaggaggatcctcaggctaaattttccactgagatagtgggaaggaaggtaatgtcatggacgaatacggaaatgataaggagagcctggcacaaaggagaaaacatgatgtattttgaacatcttatttctttgaggttttgtcagcatatttaggtgggatatccagcatgtatttgaaatataggattagaactcaagagaggtaaaacctggaagttttagttgcaaccaaaaatagttgtgatcactaagggagagcagatagagaaaaagtaagagaatgcattatacctgccccctgcccccggcaaaagactacatttatgggttaggaagaaagaagagcaagcagtggcgggggcagcagagggcagagaaacagatgagtgcagctttaccaatgccaaggttggcaggggcacaacagtatgtcagatgctgcagaatgggtggtctgggggaatgagacccgagagggctccactgaatttgatgattattgagccttaggggcactgcagacactcattttcttttcttttcttttttttttttttgagacggagtctcgtgctgtcgcccaggccggagtacagtggtgcgatctcagctcactgcaacctccacctcccgggttcaagcgattcttctgcctcagcctcccgagtagctgggattataggcacacaccactgcacctggctaattttttgtatttctagtaaagacggggtttcaccatgttggccaggctggtattgaactcttgacttcaggtaatccgcctgcctcggcctcccaagtgctaggattataggcatgagccaccacgcccggcccagacacttattttcaaagttgcagaaggggtcaaagccaaaatgcgtggaagaaaaaataaatgagtaaaataggacaagtaaaaaaatattcttttgagaaatgtgggtagtaagggagaggtgggttaaggaagtaatttctaaggtcaagacatgcaggctaaggagacgctgccagctgagtgggagagattgattgttcccctcatactcagggaagcaaggccccagggcaggtcggaggaggggaaccaaggatgctcatggagtcagccctggagattgtcagcagacattgtttctttggggtaaacaggaaaaggagagcagtgggtggcactatggagagatggggtagaatggaggacagaaaaaggtgttcacatggaaggcaaagactatctaagaagctaagaggaacagaagtggggaagacagaaaaatttgccgtgtactgcaagggcaacataataatgacatttggaggtaaagaaaagaattgctggctgggcacagtgactcatgcctgtaatcccaatacttcgggaggctgaggcaggtggatctcttgagctcaggagtttgagatcaggctgggcaatatggcaaaactctttctacagaaaatacaaaaattagcaaggcacagtggttcacgcctgtaatgccagcactttgggaggctgaggtggacagatcacttgagcccagtagttcaaaacaagcctgagcaatttgtcaagacctcatctttacaaagaattagacgagcatggtggtgcatgcatgtggttccagctccttgggaggctgaagcggaaagatcacttgagcccagatggttgaagctacagtgttcatgccgctgtactccagcctgggcaacagagcgagaccctgtctcaaaaaaaaagcggggagggggaagaaaagaaaagaattgccaagtatccctgagttgagagctgctgctgatgctgaggtttctgtgtgagtctccacccctctgcaggctgcatcagtcacctccctgcgcctgagctgcctgcagtgcacagccttggggccctagctagtcctacccccaccaggcctgacccataagcttcttttccttaataaggccctctctggcggggcacggtggctcacacctgtaatcccagcacttttggaggccgaggtgggcgtatcacaaggtcaggagtttgagaccagcctgaccaacatggtgaaacgctctctctactaaaaataaaaaattagccaggcatggtggcatgcacctgtaatcccagctactcagaaggctgaggcaggagagtcgcttgaagccggaggcggaggttgcagtgagccaagaacacgccactgcactccagcctaggcgagagagtgggactccatctcaaaaaaaaaaaaaaaaggccctctccgtgtctgttttcctaaatcgacctccttcagaggtgtcacaggacttcctgagctactttctaacaacccaagactggaatgttaaccatccactaccccacataacccttcattgctagtcacttaacggtcttccagctgctttgaaaggtactttatttgctgactcttgcagtgattttggctactaccgtccagaaaatgactccaagtgtgtggaacagccagaactgaagggccacgacctggagttttgtctgtacggaagagaagaacacctaacaacaaatgggtgaggtggccttttctcccttgtcacgatggatgaggtgttcttgttcagagtgaaagctgcttctctccacactgagatccaagccctactttagagcactatttttggcggggttggggaggggcaggatatgaaggcctccttcaagggtcaactacaggaggtgagccatgagtgcttggtgtcagaggactcaaccccaccaacaccacgaggcagagcaggagaacaagctcggccagatcctgcacccctgcctcccgcccaaccacccccaggaagggaggagaggcgagggacccacaggcagaatgacagaagcgtcccatccgccatgaacgtccatcgctcgtctggcacagtttaggcacagccagggagagctctgtctgataggactttctttggtgatggaagtgttctgtaatttgctgtgcccaatatgatagtcactagccacacatagctgtggacactagtgtaaccgaggaactgaaattttcgttttaatcaattttaagtagccaccatattggacagtgctgctctagacgggcctcattacctcgctcccctgataacttcccctctcccattttgtaccacttgttaatgagcttttttggggtcagatttctttggcacagtctcttaggtagaaagaacaggcatgtttctgacagcaggggacagagatgcttagtttgcttaaggcctgaaactacacaaaatatcccccaaaacatccctgtggacattccccccaggcctgaaacatgacgtaaggatgggaagcttttcggcagcttttcaaatcttgtttccaagaaataggcaggtacccaggaatacatccttatcttctgttaccaaatcttctttgcctggtaggtaccggaaaattccaggggacaaatgccagggtggggtaaatccagttcgagaagtaaaagacttgaaaaagaaatgcacaagcaactttttgagtccggaaaaacaggtatgttaaaataggtctagttttcaggtacaaatgggattttgtgttctgctaagaaaaatcaaagttaaagcatcaagagttttgttactgataaaaagtggtcaaactacttctctcaggcttcccttatacaggaagctgttagcatgaaggatgggtcatgtaacccagtgataagctcaccctactgaaagctcttttcttattgagggttgggggatgatattctgtaaagttgtaaaattcttaaaatcatacagttgtggaaatttctgaggctgagagtcatttaggaaaacaaaaaggttgtctgtgggctgggtacagtggctcacacctgtaatcccagcactttggaagtccaaggcaggcagatcactgaggtcaggagttcaagaccaggctgtccaacatggtgaaaccctgtctctgctaaaaatacaaaaattaaaattacaaaaattagctgggtgtggtggtttacgcctgtagtcccagttactcgggaggctgaggtaggagaatcatgtgaacctgggaggtggaggttgcagtgagccgagatcgtgctgctacactccaacctgggcaacagagcaagactccgtaccaaaaaaaaaaggttgtctgtgaagggccatgacctggaattgtgcatgtgtggaagagaagagcactgggcaataggtagggtggatctctgaccaagtcatgttaagtatgttcaaagtgaaaaattgagctatggtaatgggctggttttgatttaaatgaagcaagttacctgtcgggcatggtggcttactcctgtatagtcccagcactttgggaggctgaggtggggagatcagttgaggctatgaattggagagcagcctgggcaacatggagaaactccatctttacaaaaaaaatacaaaaattagccggatgtggtggtgtgtgcctgtagtcccagctactcaggaggctgaagggagagaatcacctgagcctgggaggtcaaggctgcagtgagccatgatcatatcactgcactccagcctgggtgacagagtgagatcctgtctcaatttaaaaaaaaaaaaaaaaaaaaaaggttatgaggctgcatccaaatctgtagcactgaactacctgtggaactggagatgtgtaaagaggaaacctcttgataatgaaacactagaccagcagcatgacttcagatgctcaacagcaggcccgtttcccaattcgtaataggagaggtcaagctggatcacctgcagtccctcctctctgctgttctaagggatgagggactgacaaggggtgggattaaggagatgatgggagaggagtgtgtgagtgtgtgtattggtaagggggcgagttcctccttctgtgcacccttgtcctgggctcctggtgactctcctcagagtgttcgtgtgtgtgattgcacaggactcccacccacagggacacagcttgtcctagaatccagctctgcctcctctgggatacattgaaaacacacactccctatttcccacccagaagcaggtaggtcacatgcaaacagtgatatgataaggcttatttataataatatataaaaaattaagtaaacaaataaatcttggtgtcatataatggcctgcctggttcttctgacacagcttcttcccagataatggccctgttctagagtcattcagcctactgttacagtagtctccctttcctactccacccttaagccccaagacttgtgaacagtggtagtgtgaagcaatcatacttgatctaggcagttgcccaggcttccagtgtaactttgacatttaaaccacagaatttgaaggttcaaggcagttggagctaagatgactctctgccagctataatattctcctggtaacactgagatggttcatcttggggggttggaaaagagccatcattaagcgcatcaggactgcagatcattcacaaagagatgaggggaatgagtggctgccttgacctcagtcctaggatatgcatgcctaaaacttttgtgtttgtgttttaatttctcaagaattccaagtcaaattctgttccaattatcctggccatcgtgggattgatgctggtcacagtcgtagcaggagtgctcattgtgaagaaatatgtctgtgggggaaggtaaggaacacagaagtcaatccaaggtactgagttcaagccagaaggctgatctagggcatatggctgagtctactcttggcaaaaactaggtacttgcagtactgttcctgaaaaatcaggcccaaatcttctcatgtcgtacatttttcatatgtcaaccatggactgtgctttctgtgtctgtgatgcgttcctgagtgtataaaactgagaaagtccacagtcctgtaagaagtgcacccctgctgtgtaccaccgagtgttaaggaggtttctgttcacaggttcctggtgcatcgatactctgtgctgcagcagcatgcagaggccaatggtgtggatggtgtggatgctttggacacagcctcccacactaataaaagtggttatcatgatgactcagatgaggtgaggctatttcttcttgaatggtagtacccccccccccaaaaaatacaagaaaataaagtctgtcttctattcatgatcaagatatgccagggccaaggcgacttaatgaccataagatttggcaagcccagaaactaggtgattgcttttgaaataggagtgttgtggtctgtcggtaatatggtgcatgtctttttcaggacctcttggaatagctcttcagaggagctggacccagcatggatggtggaaccacagtacctcttacactccctgtggctccaacttcaggaaataaatttcccattgcgagggacccagctctgtttctgctgcttccatcaaagccaaaaggacctacactaaagaaatgcagggtgggggtggggaaccctgagcacttttttacaattggctctgagaaaaagggagacattttaaattctttaacttcttatttctcgtcctgtctctttgcaaagtatgggcttttttgtttttgttttttaagggaaacgaaatggaattcgaagggaccttttcactaaccccacttctgtgtgttctgcatggcgcctgccccagggcatctgccaactccagtatcagctctcacagtgtacttggtaccatccctgggctctgctggcgagacgaaacagctgtagagatgaaaacaggctgcagaggctggcacagcctggccggcttttctccatctggggacagtcctactccaagaacactgcacaccagctcctcacacagatcccacttactctttttttttttttcagagaccacagaccacagtgatttttcttttcccttgtttaattaggcaatacccttgttaattgccctttggcaactaacttaaccatgtgcttcccacacagtacatcaggaaaacttacagggcaatatttttaacttggggcaggaagaagggagcagcagagaattgactagatatagcacctattaaaagagaactcttgcttcttctgagatttttcaagctgtgctttgtgtgtgtgccagtagacttacgcaaggacagggtacaaacttagctggaagtctgcccaggctgaatgatctcttccctagagttgattgtcgggtacacagtgtgaacccccgaagacggaacctcacagtcttccatgttcccttcttaactgtcgtgtggctcgttgctaaatcatgacaatggctgcctatctgctgcttcttaggttgctgttgtacatggaaccaggactagagattttttcagatttatagacttaaaaaattagaattttattaccaggctttccttctcaccccttttttctgactttgccaagtaatttgttgacacgaaaattttggaggaaccaattgaaaacacacttccagtctagatgatgctttgtgtgatacattaagttcttattttggattaaaagaagttttccatttgatacctctctaaattaaataaattatagaatgtagttgggtggattttggggtggccatatagtaatggaaagctgcaataattagttttaatacagcttgaatatttgctatatagaaatatagtatggaaagtttttggtcttaatgtagctactgtgcgggtcacagtttctcccaatgattatgactgggacattctttggtagataccatttgctactagtttattttgtggctagaaagtcagttttgtgtgtttttttttttttttatttgaagtgccaaattaactttagtcagaatgtgagcagatggctaagttctctcctccccagaatggattaacagctgcgtggaaagtgggggagagagtggatggagacttttagagatgttaaaactgcagtagaatgaaatgagtcagggagcttcagttagaaaataaagttgaggcagtttttgtgaagataatatggttagggctggagtgcactagtctttttgcttattcattttgcatggttttaaaattaaaaataattccgaagatacaccagctcacaaatgaaaacgtcagcctctgccccaccctccctcctgcccaaagtgaatttggtactcagaaaagaactgtttataccactcacctttctcccagcatgtactcactgtgggcagatgcaccaatacatggtaatcctcttactcattttaagacgtaggaaactcaatattcttctctaaccatatacgatagggctcttcgcttttaatgatatctgggatttctgtggaacttggcaaattttcagagcaccttcactcacataatgtcatttgaacctcacaatgttcttgggatggagtcagttgttcagggtccccgtgtgtgtgataagcagtgctggctggctgtcttcagaactcttggaaatctttacacatgcgagtgctaaccactttgagcaaggctgccttcttgtagatgacttgctgttctttatgacagggatcagtggcatttgtttcctagcagtatttagcacctttttgccaccttggtgaacagaaaattgtattttcctgtctttcatggctgaaaacaaaagtaatgggaattttaaatacgtttgcagaaactgcccctcccctcattgagggtcactgctcaagagtgcaggagtggactctccactgatgggtctccctccccatcctggtttccaccccgggctggctagctctgttggtttgaagactgacagccagcctggctcattctcattattggctagttagctttctttatcaacctgctcactcacaaatgtgtgccctcagccagagagtaagaaagcccaaatctgttacagcttctaaaaaaatagatttctaatttgtcctactcatgttaggagcattatctttgaaggtaaaacatagtgtatcattgtgtaaactcccaggcttgatgtagcagaagagatcatttctggaggcttcagcaatggaatttagcattataagagagattggacaaaccagtccaaagtggtccgagttcttaaatccaggtagggaactcactcttctttcttctctggacctaattgggcattgggctttagtgagaccacagaccaggcccgtctctcctgtaggcttttaattcaatggcaactctatttcaaagaataaaagcctttggagagttgcggcagttctgggggcgggctcaggagagtccatagatcagccgtaactggaacgtagaatctacgtctgcctctgaatggacttcccacctcctctctcttgctctgatgcttgcctctgggcctctccatgcccaaggtggtctttcatccttgacaggctggtaatgtgctggccacctccagctcctgcatcgagtctgtaaaccagagctggttctcatggccttcgtcacgataccaggatacggaggggagcccagggccatccatacccaccccagggtaacggggctggcctggcattagtcattatttagtttccaggccaaccatccagatagagattccctctttcctttgagcagtgctctcaagagctccgtgcctgtccacaatgacctagagtgcatcctgctcattgtcagtgtagcccctcgcccctatattcatccaggatacttggaagtgctaaaataggaagggattcggctttcaactttgctaccatcttccctgaagcaggaaaatgaacatggacttaaatgttctttgaaaaaaccaaagttttaagatttgctgtgtgatgaagtgacagggagggccggagtcagcaggtgccagactttctgttctgtctgccatgggtttgtccagctcaggtagctctaggagcaccatcctgccctagcagagcccaggccttgccctcatgaagcatcattgaaatagcaggagcatgttgatttcttggttaggttgcattataataacaagagtcagaacattaattcgaaacaacttgcagtatgcatttcttcacaccagtacattcttaagtgtacttgtttataaggaataacataaactaatctgtacctttatatatatgtgtgtgtacatatatacatatataaactgtatagtgtacatggtaatgatttattgctatgccccagatccttaatgtagttctcatcctccgcatgccctcagccacaagcgggtgactgactgttccctgatgatttggcccacctcctgtgtttggacctctagggaggagggttttggtcatactctccttatcctcgtgcacagaaatgctcagggtccccatgtgcctgttgttcagccctctctcttgttccctttctgagcatgtggtccttccccaggctgtgggacagctgccttcccacgaaagtgtaaagcagtattaagatcattactgcatgtgccctaaaaacccaagttttctattcccttaggacagaaaattgcatgtgaggtgggataatcgagtttcagtgacccacgtcagttacacattaaagccagaccccatgataaaattccacaaaatggaaataaaactcaaatttctttagcattgtgtaaataaatctgaatgtgtttaactttgtactggtaattttctgtatatttggaatatttgggttaaaaataaaacagactggactttgttacctgacctactgaaatgactaagctacagtttgttgatatactttttccagttttcttgcttcagaatcagactccagcaaccagagaacaagccatttaaagagccaggactgtacatttatagcatgaaatgtattcggtcatctatgaggaaaacgtctcacaaaaatgtgagccataaattacctaattttcttgttcttcattttcttgctcttagtgctcatacagatcttcctcccgtgggtgtgtatacgaccagctttcttgttcttcctcctagaaagtctaggcacccaccccacataccttcacaggtttcagttgctacactcttccttctggacacagcaacagataattactccttgaattctgtgttcttttgagcaggtttgacccctggcggcactgagaagttcagctttgtaacttcccatctgctcctcttacatttctcttgctgttcttacgggccctttcttgtccctttcatttaaggacctttggagagttgcagccttcctctcttgcaaagcatttaagagaggttcctgcccacccgtctggaaccgttagccatcctaccctgctagcctggggcaatattccatgtaatactccagcaccttggtctgaagatgacagggcaaagcccttgttcctgaggcccatctgatgggcgagttcctaccccaggacattatctaagatcgttgttttatttgaaggaattctatctaacatggtattccaagatttcctgcgccagagtcactaggttggtttttttaggtgcagatttctggatagcaccccagacctattgaattagactttctgatgggcaaaccttaggaacctgcatcttaaagttgattctttacaccttcgctccaacaaagggctaagactcagcctgggccaatttcctcaccaaaagatgggttacgtgttgtgtaaaagggggtgtatttggggtctcctggctaggagactgtccacacttacggggccttggctgggtcaagcaggaaaaatagttcagaaagaccttcaagctagttctgatatggcaaaaacccagtcttctgccaaccaaatcaaccccataggccatccaaggccccaataaacagattgtcctgggccttagctcctataccagttacaaagaggctgagcttctgtcaccccaagataacacaggctcctgggatcttaccaattaccccaaaattgaaggcaaaaatgagtaagaagtcagtccatttgttcaactgcacccattttttttccatgaaaaaaatacaggggtgagtagggggctgtgggaaccctatactttccactcagctttgccgtgactctcaaactgctctaaaattaaagcattttaaaatgcagagggatgctggcttctggagataagaaggtaggagtatgataggtggcagtgtccatggcatgtacaacacgtatagaaagatggtttgctaagtctggaaatcatgtaggacctgtagtatgtgactagcacctgctgtgagctggccactttcctatagattgtatcattttatcctcaacacaatcatggcatcatcccttgtttcatagactgggacttgagactccgagtcggccatttgcccagagtcccagctggtgagtgagtggtacagccagaagtcacacgcaggtctctgagctcgcctccctccaccactgctgcctctccctttatttgtacttcatctaataggcaaggcagccattgacagctctcaaagggggaagtgctggcctgacctaaaaatagcttacatttatcaagcacttagtcggctacactctgctaagcactttacagagattacctctttaatcctcacaacgaccctatggttataattatctccattttacagatgaggaaacagactgaagagagactgtgaggtgttgtgagtcacacagctagttggtgacagagccccaagtggcacctggg

SEQ ID NO: 4 - protein, 6 aa, synthetic construct
  REGION 1..6: note = Description of Artificial Sequence: Synthetic 6xHis tag
HHHHHH

SEQ ID NO: 5 - protein, 8 aa, synthetic construct
  REGION 1..8: note = Description of Artificial Sequence: Synthetic peptide
DYKDDDDK

SEQ ID NO: 6 - protein, 4 aa, Homo sapiens
RWRR